SEQUENCE LISTING (38 sequences)

SEQ ID NO: 1 - DNA, 20 bp, Artificial Sequence
  Synthetic oligonucleotide
tgcttccctgagacccagtt

SEQ ID NO: 2 - DNA, 25 bp, Artificial Sequence
  Synthetic oligonucleotide
gatcacttctttcctttgcatcaag

SEQ ID NO: 3 - DNA, 20 bp, Artificial Sequence
  Synthetic oligonucleotide
gggctctctgagaggcaggt

SEQ ID NO: 4 - DNA, 19 bp, Artificial Sequence
  Synthetic oligonucleotide
cctttgctctgcggttctg

SEQ ID NO: 5 - DNA, 19 bp, Artificial Sequence
  Synthetic oligonucleotide
ggattcccagagcccacaa

SEQ ID NO: 6 - DNA, 21 bp, Artificial Sequence
  Synthetic oligonucleotide
acatccactggcagtacagaa

SEQ ID NO: 7 - DNA, 18 bp, Artificial Sequence
  Synthetic oligonucleotide
gggagcggtgaagatgga

SEQ ID NO: 8 - DNA, 22 bp, Artificial Sequence
  Synthetic oligonucleotide
tcatgttgctcacggaggagta

SEQ ID NO: 9 - DNA, 22 bp, Artificial Sequence
  Synthetic oligonucleotide
atcggctacaactacacctaca

SEQ ID NO: 10 - DNA, 21 bp, Artificial Sequence
  Synthetic oligonucleotide
gtacatgctgcacaggaagaa

SEQ ID NO: 11 - DNA, 20 bp, Artificial Sequence
  Synthetic oligonucleotide
tccctcttccctcctcaaat

SEQ ID NO: 12 - DNA, 20 bp, Artificial Sequence
  Synthetic oligonucleotide
tcagcgtgtaaaggcatctg

SEQ ID NO: 13 - DNA, 20 bp, Artificial Sequence
  Synthetic oligonucleotide
cacccgacgcccttttacat

SEQ ID NO: 14 - DNA, 19 bp, Artificial Sequence
  Synthetic oligonucleotide
gaaggctggatggatcggc

SEQ ID NO: 15 - DNA, 20 bp, Artificial Sequence
  Synthetic oligonucleotide
ggtaccccgacatccacttg

SEQ ID NO: 16 - DNA, 20 bp, Artificial Sequence
  Synthetic oligonucleotide
taatctccggcctagccaaa

SEQ ID NO: 17 - DNA, 25 bp, Artificial Sequence
  Synthetic oligonucleotide
agaactctccaacatcctgaacctc

SEQ ID NO: 18 - DNA, 20 bp, Artificial Sequence
  Synthetic oligonucleotide
ctgaggccttctgcgtcaca

SEQ ID NO: 19 - DNA, 21 bp, Artificial Sequence
  Synthetic oligonucleotide
acttactggccagggctacac

SEQ ID NO: 20 - DNA, 19 bp, Artificial Sequence
  Synthetic oligonucleotide
acttcccggcacctctgaa

SEQ ID NO: 21 - DNA, 20 bp, Artificial Sequence
  Synthetic oligonucleotide
agtgagaggcaacctggaga

SEQ ID NO: 22 - DNA, 20 bp, Artificial Sequence
  Synthetic oligonucleotide
acactcggaccacatccttc

SEQ ID NO: 23 - DNA, 19 bp, Artificial Sequence
  Synthetic oligonucleotide
aaaccaaaggtcgccacac

SEQ ID NO: 24 - DNA, 21 bp, Artificial Sequence
  Synthetic oligonucleotide
gttgatggaactgacgctagg

SEQ ID NO: 25 - DNA, 23 bp, Artificial Sequence
  Synthetic oligonucleotide
tctccaatctgaaggtccacctg

SEQ ID NO: 26 - DNA, 25 bp, Artificial Sequence
  Synthetic oligonucleotide
gattgctggtgctgctaaatctctt

SEQ ID NO: 27 - DNA, 25 bp, Artificial Sequence
  Synthetic oligonucleotide
ttcctttactgaagggagacgaagg

SEQ ID NO: 28 - DNA, 25 bp, Artificial Sequence
  Synthetic oligonucleotide
ccatccaaaggaatcgtgccataaa

SEQ ID NO: 29 - DNA, 20 bp, Artificial Sequence
  Synthetic oligonucleotide
cgcacggaatttgaacagta

SEQ ID NO: 30 - DNA, 20 bp, Artificial Sequence
  Synthetic oligonucleotide
ggatcagggacctgtcacac

SEQ ID NO: 31 - DNA, 19 bp, Artificial Sequence
  Synthetic oligonucleotide
tggacagttacgcgcacat

SEQ ID NO: 32 - DNA, 21 bp, Artificial Sequence
  Synthetic oligonucleotide
cgagtaggacatgctgtaggt

SEQ ID NO: 33 - DNA, 22 bp, Artificial Sequence
  Synthetic oligonucleotide
attaagaggatgctgggctctg

SEQ ID NO: 34 - DNA, 22 bp, Artificial Sequence
  Synthetic oligonucleotide
cactgtactgcacactcacctt

SEQ ID NO: 35 - DNA, 22 bp, Artificial Sequence
  Synthetic oligonucleotide
agctcaaagttgtcctactgcc

SEQ ID NO: 36 - DNA, 22 bp, Artificial Sequence
  Synthetic oligonucleotide
ttctaacccaagcacagatccc

SEQ ID NO: 37 - DNA, 19 bp, Artificial Sequence
  Synthetic oligonucleotide
gtgccctatgtgccgttac

SEQ ID NO: 38 - DNA, 19 bp, Artificial Sequence
  Synthetic oligonucleotide
ggctgacgttggactgagg

(19) United States
(12) Patent Application Publication
(10) Pub. No.: US 2023/0242871 A1
(43) Pub. Date: Aug. 3, 2023

(54) GENERATION OF PRIMORDIAL GERM CELLS AND METHODS OF USING THE SAME

(71) Applicant: The Board of Trustees of the Leland Stanfor Junior University, Palo Alto, CA (US)

(72) Inventors: Vittorio Sebastiano, Palo Alto, CA (US); Kyle M. Loh, Palo Alto, CA (US); Gugene Kang, Palo Alto, CA (US)

(21) Appl. No.: 18/000,072

(22) PCT Filed: May 28, 2021
(86) PCT No.: PCT/US2021/034925

§ 371 Date: Nov. 28, 2022

Related U.S. Application Data
(60) Provisional application No. 63/032,382, filed on May 29, 2020.

(51) Int. Cl. C12N 5/0735

(52) U.S. Cl. CPC C12N 5/0611; C12N 2501/11; C12N 2501/15; C12N 2501/125; C12N 2501/155; C12N 2501/235; C12N 2501/415; C12N 2506/45

(57) ABSTRACT

Provided herein are, inter alia, compositions and methods for generating a population of PGCs with improved efficiencies and line-to-line consistency. Inductive signals are temporally dynamic: WNT activation for 12 hours incipiently differentiates primed hPSCs into posterior epiblast, while subsequently, sharp WNT inhibition together with BMP activation specifies PGCs. hPSC-derived PGCs can be easily purified by virtue of their CXCR4⁺PDGFRα⁻GARP-surface-marker profile.

### CROSS-REFERENCE TO RELATED APPLICATIONS

This application claims priority to U.S. Provisional Application No. 63/032,382, filed May 29, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under contract OD024558 awarded by the National Institutes of Health. The government has certain rights in the invention.

### REFERENCE TO A SEQUENCE LISTING, A TABLE OR A COMPUTER PROGRAM LISTING APPENDIX SUBMITTED AS AN ASCII TEXT FIL TABLE

The Sequence Listing written in file 041243-558001WO_ST25.txt, created on May 26, 2021, 8,192 bytes, machine format IBM-PC, MS Windows operating system, is hereby incorporated by reference.

### BACKGROUND OF THE INVENTION

At least three separate origins for Primordial Germ Cells (PGC) have been proposed. First, naive pluripotent stem cells (PSCs) have been suggested to uniquely possess germline potential, on the basis of studies that have found that naive mouse (Hayashi et al., 2011) and human (Irie et al., 2014) PSCs could produce PGC-like cells in vitro, whereas primed PSCs could not. Second, other studies have found that human primed PSCs can also differentiate into PGC-like cells (Kobayashi et al., 2017; Sasaki et al., 2015), arguing against the hypothesis that nave PSCs are uniquely privileged. Third, analyses of monkey embryos have suggested that the amnion is an alternative origin for PGCs (Sasaki et al., 2016).

Independent of their proposed origins, the precise lineage path that pluripotent cells take en route to become PGCs has also remained controversial. PGCs were historically thought to originate directly from pluripotent epiblast cells without transition through a somatic-like intermediate. Yet, Brachyurya master regulator of the primitive streak (PS, the embryonic precursor to definitive endoderm and mesoderm) is critical for PGC specification in mouse embryos (Aramaki et al., 2013). Together with the origins of mouse PGCs from the proximal posterior epiblast in the vicinity of the posterior PS (Ginsburg et al., 1990; Lawson and Hage, 1994) and reports that mesoderm-like cells can generate PGCs in vitro (Sasaki et al., 2015), this suggested that PGCs might have a PS-like provenance. A recent report suggested that in vivo, epiblast first matures into posterior epiblast (the precursor to the PS) but that posterior epiblast cells then branch off to give rise to either PGCs or the PS (Kobayashi et al., 2017). In this model, PGCs and PS are distinct lineages but they share a common origin in the posterior epiblast (Kobayashi et al., 2017).

Given the uncertainties surrounding human PGC origins, preceding methods to generate human PGC-like cells from PSCs generated heterogeneous cell populations containing only a subset of PGC-like cells relied on complex 3D cultures with high concentrations of growth factors and the presence of undefined components like Knockout Serum Replacer (Irie et al., 2014; Kobayashi et al., 2017; Sasaki et al., 2015); and demonstrated broad line-to-line variability (Yokobayashi et al., 2017). Without the use of transgenic reporters it can be difficult to distinguish PGC-like cells from other commingled fates, as SOX17a marker for human PGCs (Irie et al., 2014) is also expressed in definitive endoderm (Kanai-Azuma et al., 2002) and blood progenitors (Clarke et al., 2013).

The development of a robust and efficient in vitro platform to generate PGCs from human pluripotent stem cells would enable modeling germ cells development and have implications in treatment of infertility. Provided herein, inter alia, are solutions to these and other problems in the art.

### BRIEF SUMMARY OF THE INVENTION

In an aspect is provided a method of forming a primordial germ cell (PGC) in vitro, the method including: (i) contacting a pluripotent stem cell population with a wingless integrated (WNT) agonist and a transforming growth factor beta (TGFβ) agonist, thereby forming a posterior epiblast cell population; (ii) contacting the posterior epiblast cell population with a WNT inhibitor and removing the WNT agonist and the TGFβ agonist, thereby forming a PGC.

In an aspect a method of isolating a primordial germ cell (PGC) is provided, the method including: (i) contacting a pluripotent stem cell population with a WNT agonist and a TGFβ agonist in vitro, thereby forming a posterior epiblast cell population; (ii) contacting the posterior epiblast cell population with a WNT inhibitor and removing the WNT agonist and the TGFβ agonist; thereby forming a cell population including a PGC; and (iii) separating a CXCR4^{+/}PDGFRα⁻/GARP cell from the cell population including a PGC, thereby isolating the PGC.

In an aspect a method of forming a primordial germ cell (PGC) in vitro is provided, The method includes (i) contacting a pluripotent stem cell population with a wingless integrated (WNT) agonist and a transforming growth factor beta (TGFβ) agonist, thereby forming a posterior epiblast cell population; (ii) contacting the posterior epiblast cell population with a WNT inhibitor, wherein prior to the contacting of step (ii) the WNT agonist and the TGFβ agonist are removed, thereby forming a PGC.

In an aspect a method of isolating a primordial germ cell (PGC) is provided. The method includes, (i) contacting a pluripotent stem cell population with a WNT agonist and a TGFβ agonist in vitro, thereby forming a posterior epiblast cell population; (ii) contacting the posterior epiblast cell population with a WNT inhibitor, wherein prior to the contacting of step (ii) the WNT agonist and the TGFβ agonist are removed, thereby forming a cell population comprising a PGC; and (iii) separating a CXCR4^{+/}PDGFRα⁻/GARP cell from the cell population comprising a PGC, thereby isolating said PGC.

In an aspect a method of treating infertility in a subject in need thereof is provided, the method including administering a therapeutically effective amount of a PGC provided herein including embodiments thereof to the subject, thereby treating infertility in the subject.

In an aspect, a primordial germ cell formed by a method provided herein including embodiments thereof is provided.

In an aspect is provided an in vitro cell composition, including a WNT-activated posterior epiblast cell population in a cell culture medium including a WNT inhibitor.

In an aspect, an in vitro cell culture, including a WNT-activated posterior epiblast cell population in a cell culture medium including a WNT inhibitor is provided.

In an aspect, an in vitro cell culture, including a CXCR4+/PDGFRα-/GARP¬ PGC in a cell culture medium including a Wnt inhibitor is provided.

In an aspect, a primordial germ cell in a cell culture container including a cell culture medium including a WNT inhibitor is provided, wherein the PGC is a CXCR4+/PDGFRα-/GARP¬ PGC.

### BRIEF DESCRIPTION OF THE DRAWINGS

FIGS. 1A-1J. FIG. 1A illustrates unresolved issues regarding PGC origins. The figure is a schematic of proposed steps of PGC development in early embryogenesis and biological questions; depicted cell positions are based on pig embryos (Kobayashi et al., 2017). FIG. 1B illustrates a comparison of earlier differentiation strategies. The figure shows flow cytometry analysis of fluorescent reporter expression during differentiation of WIS1 NANOS3-mCherry hESCs and H9 SOX17-GFP hESCs differentiated using previously-published protocols (Kobayashi et al., 2017; Sasaki et al., 2015). FIG. 1C illustrates exposure to primitive streak-inducing signals for 12 hours is optimal for subsequent PGC specification; NANOS3-mCherry hESCs were exposed to primitive streak-inducing signals (Activin + CHIR + Y27632) for either 6, 12, or 48 hours (phase I), and then transferred to PGC-specifying media for 3 days (phase II), and flow cytometry was then performed. FIG. 1D illustrates the molecular description of the posterior epiblast state by single-cell RNA-sequencing. The figure shows a t-SNE plot of single-cell RNA-sequencing data of posterior epiblast cells (D0.5 of differentiation) and hPSCs (D0 of differentiation) showing expression of pluripotency markers OCT4/POU5F1 and SOX2 and primitive streak marker MIXLl. FIG. 1E illustrates marker expression in posterior epiblast vs. primitive streak. The figure shows a bar graph of qPCR analysis of D0 (hPSC), D0.5 (posterior epiblast) and D1 (primitive streak) of differentiation showing expression of pluripotency and primitive streak markers; qPCR data were normalized to the sample with the highest expression (which was set = 1.0); error bars = standard error. FIG. 1F illustrates temporally dynamic WNT specifies PGC. The figure shows a schematic (top panel) and bar graph (bottom panel) showing WNT inhibition promotes differentiation of posterior epiblast into PGCs; NANOS3-mCherry hESCs were differentiated into posterior epiblast using a WNT agonist (12 hours, phase I), and then were transferred into PGC-specifying media for 3 days in the presence of WNT agonist (CHIR) or WNT inhibitor (XAV939) (phase II); and finally, flow cytometry was then performed. FIG. 1G illustrates new signaling strategy. The figure shows a schematic of the 2D monolayer PGC differentiation protocol described herein. FIG. 1H illustrates generation of NANOS3+ PGCs in monolayer conditions. The figure shows flow cytometry analysis of NANOS3-mCherry hESC shows fluorescent reporter expression before or after 3.5 days of differentiation. FIG. 1I illustrates hPSC-derived NANOS3+ PGCs express PGC markers. The figure shows bar graphs of qPCR analysis of NANOS3-mCherry+ PGCs andNANOS3-mCherry- non-PGCs derived after 3.5 days of differentiation, as shown in (FIG. 1H); as a negative control, undifferentiated hPSCs (D0) were also analyzed, and gene expression is shown relative to undifferentiated hPSCs (which was set = 1.0). FIG. 1J illustrates hPSC-derived D3.5 differentiated populations express PGC marker proteins. The figure shows representative images of immunostaining of hPSCs differentiated for D3.5 showing expression of PGC markers in a subset of cells (nuclear counterstain: DAPI).

FIGS. 2A-2C. FIG. 2A illustrates hPSC-derived NANOS3+ PGCs are CXCR4+ GARP- PDGFRα-. The figure shows flow cytometry analysis of D3.5 differentiated NANOS3-mCherry hESCs reveals CXCR4, GARP and PDGFRα expression relative to NANOS3-mCherry fluorescent reporter expression. FIG. 2B illustrates CXCR4+ GARP-PDGFRα- surface marker profile enables purification of hESC-derived PGCs without genetic reporters. The figure shows flow cytometry gating strategy to identify CXCR4⁺/GARP⁻/ PDGFRα⁻ PGCs derived from H9 hESCs (that did not carry any fluorescent reporters) that were differentiated for D3.5; various cell populations from the D3.5 population were FACS sorted and subject to qPCR analysis, revealing that pluripotency and PGC markers are restricted to the CXCR4⁺/GARP⁻/ PDGFRα⁻ subset and therefore reaffirming its PGC identity. FIG. 2C illustrates validation of FACS-purified CXCR4+ GARP- PDGFRA-PGCs. The figure shows representative images of immunostaining validation of FACS-purified CXCR4⁺/GARP⁻/ PDGFRα⁻ hPSC-derived PGCs confirms that they ubiquitously express PGC marker proteins (nuclear counterstain: DAPI).

FIGS. 3A-3B. FIG. 3A illustrates comparison of PGC differentiation strategies and reproducibility across multiple hESC/hiPSC lines. The figure shows multiple hESC/hiPSC lines (H1, H9, BJC1, BJC3, BIRc3) were differentiated into PGCs using our protocol or using previously-published differentiation protocols (Kobayashi et al., 2017; Sasaki et al., 2015) in side-by-side comparisons; then, flow cytometry analysis to quantify the purity of CXCR4⁺/GARP⁻/PDGFRα⁻ PGCs was performed (data summarized in the left-hand histogram). FIG. 3B illustrates reproducible, consistent PGC formation from multiple hESC/hiPSC lines. To confirm that PGC differentiation is robust across multiple genetic backgrounds, multiple hESC lines (H1, H9, WIS1 NANOS3-mCherry) and hiPSC lines (BJC1, BJC3, BIRc3), were differentiated for D3.5, and then qPCR was performed on either the bulk D3.5 population or the FACS-purified D3.5 PGC population (which FACS sorted for CXCR4⁺/GARP⁻/ PDGFRα- cells in the H1, H9, BJC1, BJC3, BIRc3 lines, or for NANOS3-mCherry expression in the NANOS3-mCherry line). qPCR data were normalized to expression in undifferentiated cells (which was set =1.0).

FIGS. 4A-4F. FIG. 4A illustrate single-cell transcriptional analysis of germ cell differentiation. The figure shows a schematic of stages profiled for single cell RNA-sequencing (scRNAseq): D0 hPSCs, D0.5 posterior epiblast, D3.5 bulk population, D3.5 FACS-sorted CXCR4⁺/GARP⁻/ PDGFRα⁻ PGCs and D2 definitive endoderm (left); t-SNE projection of the combined scRNAseq data sets, where single cells are colored by their cluster annotation (right). FIG. 4B illustrates D3.5 bulk population is heterogeneous, comprises PGCs and mesoderm-like cells. The figure shows t-SNE projection of hPSC-derived D3.5 bulk population shows that it is heterogeneous and segregates into 2 major clusters: a PGC cluster expressing PGC markers (TFAP2C, KLF4, NANOS3) and mesoderm-like cells (non-PGCs) expressing mesoderm markers (HAND1, TMEM88, MYL4). FIG. 4C illustrates D3.5 non-PGCs are mesoderm-like cells. The figure shows representative images of immunostaining of hPSC-derived D3.5 bulk population confirms that it is heterogeneous, comprising a mixture of PGCs (SOX17⁺, NANOG⁺) and non-PGCs (HAND1⁺) (nuclear counterstain: DAPI). FIG. 4D illustrates CXCR4+ GARP- PDGFRA- FACS sorting isolates nearly-pure PGCs. The figure shows t-SNE projection of scRNAseq data from hPSC-derived FACS-sorted D3.5 CXCR4⁺/GARP⁻/ PDGFRα⁻ PGCs shows that is largely uniform, with the predominant cluster (comprising 97.2% of sorted cells) expressing PGC markers (NANOS3 and TFAP2C). FIG. 4E illustrates marker expression and cellular diversity across germ cell differentiation and in endoderm cells by single-cell RNA-sequencing. The figure shows violin plots of scRNAseq data shows expression of posterior epiblast, pluripotency, lateral mesoderm, cardiac, PGC and naive pluripotency markers across the 5 different cell-types (clusters) identified from the combined scRNAseq dataset (comprising merged D0, D0.5 posterior epiblast, D3.5 bulk, D3.5 FACS-sorted PGCs and definitive endoderm scRNAseq datasets). FIG. 4F illustrates continuous pluripotency factor expression during germline differentiation. The figure shows different models for pluripotency transcription factor expression during hPSC differentiation into PGCs (i, top left); scRNAseq reconstruction of differentiation trajectory of hPSCs progressing into D3.5 PGCs and non-PGCs, showing pluripotency and PGC markers expression as a function of pseudotime (ii, bottom); flow cytometry analysis of NANOG-2A-YFP knock-in reporter hPSCs differentiating towards D3.5 PGCs, showing consistent NANOG expression throughout differentiation (iii, top right).

FIGS. 5A-5H. FIG. 5A illustrates single-cell RNA-seq comparison of hPSCs vs. posterior epiblast. The figure shows t-SNE plots of D0 hPSC vs D0.5 posterior epiblast scRNAseq datasets showing expression of posterior epiblast markers. FIG. 5B illustrates hPSC vs. posterior epiblast comparison. The figure shows violin plots of D0 hPSC vs D0.5 posterior epiblast scRNAseq datasets showing expression of genes enriched in posterior epiblast vs. hPSCs. FIG. 5C illustrates SCF and EGF are dispensable on the D0.5-D1.5 interval. NANOS3-mCherry hPSCs were first differentiated into posterior epiblast (ACY = Activin + CHIR + Y27632 for 12 hrs); subsequently, for posterior epiblast differentiation into PGCs in the context of BMP4 + SCF + EGF + XAV939 + Y-27632 from D0.5-3.5, BMP4 subtraction from D1.5-2.5 and SCF/EGF subtraction from D0.5-1.5 both enhance efficiency, as assayed by flow cytometry analysis for NANOS3-mCherry fluorescent reporter expression on D3.5. FIG. 5D illustrates low BMP doses needed for monolayer differentiation. The figure shows BMP4 can be titrated to 20 ng/mL on D0.5-3.5 without significant loss in efficiency, as assayed by flow cytometry analysis for NANOS3-mCherry fluorescent reporter expression on D3.5. FIG. 5E illustrates LIF is dispensable for the second differentiation stage. The figure shows addition of LIF during D0.5-3.5 does not improve efficiency, as assayed by flow cytometry analysis for NANOS3-mCherry fluorescent reporter expression on D3.5. FIG. 5F illustrates 3 days are optimal for the second differentiation stage. The figure shows in our hPSC-to-PGC differentiation protocol, mCherry expression peaks on D3.5 of differentiation, as assayed by flow cytometry analysis for NANOS3-mCherry fluorescent reporter expression on various timepoints. FIG. 5G illustrates hPSC-derived NANOS3-mCherry+ PGCs do not express other markers. The figure shows qPCR analysis of NANOS3-mCherry+ PGCs and NANOS3-mCherry- non-PGCs derived after 3.5 days of differentiation shows expression of mesoderm and endoderm markers; as a negative control, undifferentiated hPSCs (D0) were also analyzed, and gene expression is shown relative to undifferentiated hPSCs (which was set = 1.0). FIG. 5H illustrates generating SOX17-GFP+ PGCs from hPSCs. H9 SOX17-GFP hESCs were differentiated into PGCs and flow cytometry was performed to assay reporter expression before or after 3.5 days of differentiation (left); to confirm that SOX17-GFP⁺ D3.5 cells are PGCs, qPCR was performed on D3.5 SOX17-GFP⁺ and SOX17-GFP⁻ cells for PGC marker NANOS3 and definitive endoderm marker FOXA2; as a negative control, undifferentiated hPSCs (D0) were also analyzed, and gene expression is shown relative to undifferentiated hPSCs (which was set = 1.0) (right).

FIGS. 6A-6C. illustrate loss of one SOX17 allele in the SOX17-GFP reporter line partially impairs PGC specification. FIG. 6A shows comparison of wild-type H9 hESCs vs. SOX17-GFP hESCs (where one SOX17 allele is replaced with GFP, thus generating functionally SOX17-heterozygous cells (Wang et al., 2011)). FIG. 6B shows comparison of wild-type H9 hESCs vs. SOX17-GFP hESCs differentiated into PGCs. FIG. 6C shows comparison of wild-type H9 hESCs vs. SOX17-GFP hESCs, where a subset of the cells were differentiated into extraneous lineages. qPCR was performed on undifferentiated hESCs (D0), D3.5 bulk populations and D3.5 FACS-purified PGCs in the 2 genetic backgrounds; gene expression is shown relative to undifferentiated hPSCs (which was set = 1.0).

FIGS. 7A-7B. illustrate PGCs generated from multiple hPSC lines display appropriate marker expression. To confirm that PGC differentiation is robust across multiple genetic backgrounds, multiple hESC lines (H1, H9, WIS1 NANOS3-mCherry) and hiPSC lines (BJC1, BJC3, BIRc3), were differentiated for D3.5, and then qPCR was performed on either the bulk D3.5 population or the FACS-purified D3.5 PGC population (which FACS sorted for CXCR4⁺/GARP⁻/ PDGFRα⁻ cells in the H1, H9, BJC1, BJC3, BIRc3 lines, or for NANOS3-mCherry expression in the NANOS3-mCherry line). qPCR data were normalized to expression in undifferentiated cells (which was set = 1.0). FIG. 7A shows across cell lines tested, hPSC-derived CXCR4⁺ PDGFRA⁻ GARP⁻ PGCs demonstrated upregulation of hallmark PGC markers FIG. 7B shows that the cell lines tested did not have substantial expression of markers indiating differentiation to extraneous lineages.

FIGS. 8A-8D. FIG. 8A illustrate quality metrics of single-cell RNA-seq data. The figure shows violin plot of genes detected, unique molecular identifier (UMI) counts, percent mitochondrial reads and percent ribosomal reads across all 5 cell-type clusters (ES = undifferentiated hESCs; PE = posterior epiblast; En = endoderm; Me = mesoderm [non-PGC]). FIG. 8B illustrates transcriptionally assigning cell-types from scRNAseq data. The figure shows the proportion of cells from different libraries in each cluster; N.B. PGCs are found in both the D3.5 bulk as well as the D3.5 FACS-sorted PGC samples, whereas mesoderm cells are found almost exclusively in the D3.5 bulk population but not the D3.5 FACS-sorted PGC samples. FIG. 8C illustrates mutually-exclusive germ vs. mesoderm marker expression. The figure shows t-SNE plot with overlaid expression of PGC and mesoderm markers showing that these markers are expressed in a mutually-exclusive fashion in different cell clusters. FIG. 8D shows a heatmap of top differentially expressed genes across the 5 major clusters (i.e., cell-types).

FIGS. 9A-9C. FIG. 9A illustrate PGC marker expression in hPSC-derived PGCs and other cell-types. The figure shows violin plots of scRNAseq data shows expression of PGC/naïve pluripotency markers across the 5 different cell-types (clusters) identified from the combined scRNAseq dataset (comprising merged D0, D0.5 posterior epiblast, D3.5 bulk, D3.5 FACS-sorted PGCs and definitive endoderm scRNAseq datasets). FIG. 9B illustrates bifurcation of NANOG+ hPSCs/posterior epiblast into NANOG+ CXCR4+ PGCs vs. NANOG- CXCR4- non-PGCs. The figure shows flow cytometry analysis of NANOG-2A-YFP hPSCs differentiated towards PGCs, with surface staining of CXCR4 combined with either flow cytometry analysis of NANOG-2A-YFP reporter activity or else intracellular staining of NANOG protein itself. FIG. 9C illustrates NANOG protein continuously expressed during germ cell differentiation. The figure shows Intracellular flow cytometry for NANOG protein expression during hPSC differentiation towards PGCs.

FIGS. 10A-10D: illustrate optimization of WNT activation in hPSCs. Data shows that 6-12 hrs of WNT activation elicits efficient PGCs formation (assessed at d3.5 by CXCR4+/PDGFRalpha-/GARP-) with most optimal and reproducible yields at 12 hrs. FIG. 10A shows detection of CXCR4+/PDGFRalpha-/GARP- from H1 cells, FIG. 10B shows detection of CXCR4+/PDGFRalpha-/GARP- from H1 cells; FIG. 10C shows detection of CXCR4+/PDGFRalpha-/GARP- from NANOG-YFP knock-in H9 cells; CXCR4+/PDGFRalpha-/GARP- cells were detected at 6 hrs, 12 hrs and 24 hrs. FIG. 10D illustrates flow cytometry analysis of the cells. The graphs of FIGS. 10A-10C are quantification of FACS plots in FIG. 10D.

FIGS. 11A-11C. illustrate quantification of hPGCLCs at d3.5 by triple staining for SOX17/BLIMP1/NANOG (unsorted). FIG. 11A is a graph showing the abundance of cells expressing SOX17 and BLIMP1 (top panel) or NANOG and BLIMP1 (bottom panel). FIG. 11B are representative images showing triple staining of H9 cells (left) and H1 cells (right) for SOX17/BLIMP1/NANOG. FIG. 12C is a graph showing the percentage of H9 or H1 cells that were positive for SOX17/BLIMP1/NANOG at d3.5.

FIGS. 12A-12B. are representative images showing expression of 5hmC in unsorted hPGCLCs at d3.5 in H9 cells. The images show that hPGCLCs (AP2gamma and SOX17 double positive cells) express higher levels of 5hmC. FIG. 12A shows images with the scale bar scale bar at 100 um. FIG. 12B is a magnified version of FIG. 12A with the scale bar at 50 um.

FIGS. 13A-13B. are representative images illustrating expression of 5hmC in unsorted hPGCLCs at d3.5 in H1 cells. The images indicate that hPGCLCs (AP2gamma and SOX17 double positive cells) express higher levels of 5hmC. FIG. 13A shows images with the scale bar at 100 um. FIG. 13B is a magnified version of FIG. 13A with the scale bar at 50 um.

FIG. 14. illustrates quantification of hPGCLCs. The cells were quantified by immunofluorescence triple staining staining at D3.5 for SOX17/BLIMP1/NANOG (sorted).

FIG. 15. illustrates imaging of NANOG-YFP expression throughout differentiation into hPGCLCs. Representative images were taken at day 0.5, 1.5, 2.5 and 3.5. The images illustrate increased expression of NANOG as cell differentiation progresses.

FIGS. 16A-16E. are representative images of cells stained for NANOG from d0 to d3.5. A subset of cells maintains expression of NANOG and OCT4 throughout the differentiation and becomes progressively positive for SOX17. FIG. 16A shows images of H9 hESCs at D0, FIG. 16B shows the cells at D0.5, FIG. 16C shows the cells at D1.5, FIG. 16D shows the cells at D2.5 and FIG. 16E shows the cells at D3.5.

### DETAILED DESCRIPTION OF THE INVENTION

Provided herein are compositions and methods for generating a simplified, monolayer to generate early PGCs within 3.5 days, with improved efficiencies and line-to-line consistency compared to existing methods. Inductive signals are temporally dynamic: WNT activation for 12 hours incipiently differentiates primed hPSCs into posterior epiblast, while subsequently, sharp WNT inhibition together with BMP activation specifies PGCs. hPSC-derived PGCs can be easily purified by virtue of their CXCR4+PDGFRA-GARP-surface-marker profile.

Compositions and methods provided herein may be used for in vitro generation of human Primordial Germ Cell; treatment of male and female infertility; production of autologous sperm and oocytes; modeling of infertility; platform to develop new drugs for the treatment of infertility.

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/- 10% of the specified value. In embodiments, about means the specified value.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded RNA, and hybrid molecules having mixtures of single and double stranded DNA and RNA. Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In embodiments, the nucleic acid is messenger RNA. In embodiments, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art. In embodiments, the phosphorothioate nucleic acid or phosphorothioate polymer backbone is connected to a detectable label through a chemical linker.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. A nonspecific sequence may be a sequence that does not encode for a functional nucleic acid or protein. In embodiments, a nonspecific sequence is a sequence of a nucleic acid that includes nucleotides randomly attached to each other. In embodiments, a nonspecific sequence does not encode for a biological function. A a nonspecific sequence may be referred to as a “scrambled” sequence (e.g., scrambled nucleic acid sequence). A scrambled sequence (e.g., scrambled nucleic acid sequence) may be created by a software tool to create the sequence scramble as negative control for a functional sequence (e.g., nucleic acid sequence). By way of example, a nonspecific sequence (e.g., nucleic acid sequence) is a sequence (e.g., nucleic acid sequence) that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5x SSC, and 1% SDS, incubating at 42° C., or, 5x SSC, 1% SDS, incubating at 65° C., with wash in 0.2x SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1X SSC at 45℃. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, In embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein (e.g., TGFβ, BMP4, CXCR4, PDGFRα, SCF, EGF, etc.), the named protein includes any of the protein’s naturally occurring forms, or variants or homologs that maintain the protein activity (e.g., within at least 50%, 80%, 90%, 95%, 96%, 97%, 98%, 99% or 100% activity compared to the native protein). In embodiments, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the protein is the protein as identified by its NCBI sequence reference. In embodiments, the protein is the protein as identified by its NCBI sequence reference or functional fragment or homolog thereof.

The term “PDGFRα” as provided herein includes any of the alpha-type platelet-derived growth factor receptor (PDGFRα) protein naturally occurring forms, homologs or variants that maintain the tyrosine kinase activity of PDGFRα (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the PDGFRα protein is the protein as identified by the NCBI sequence reference GI:5453870. In embodiments, the PDGFRα protein is the protein as identified by the NCBI sequence reference GL5453870, homolog or functional fragment thereof. In embodiments, the PDGFR-a protein is encoded by a nucleic acid sequence corresponding to Gene ID: GI: 172072625.

The term “CXCR4” as provided herein includes any of the C-X-C motif chemokine receptor 4 (CXCR4) protein naturally occurring forms, homologs or variants that maintain the activity of CXCR4 (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the CXCR4 protein is the protein as identified by the UniProt sequence reference P61073, homolog or functional fragment thereof.

The terms “BMP” and “bone morphogenetic protein” refer to any BMP protein (including homologs, isoforms, and functional fragments thereof) with BMP activity. The term includes any recombinant or naturally-occurring form of BMP or variants, homologs, or isoforms thereof that maintain BMP activity (e.g. within at least 30%, 40%, 50%, 60%, 70%, 80%, 90%, 95%, or 100% activity compared to wildtype BMP). In some aspects, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring BMP protein. In embodiments, the BMP protein is substantially identical to the protein identified by Accession No. NP_001191 or variants, homologs, or isoforms having substantial identity thereto. In embodiments, the BMP protein is substantially identical to the protein identified by UniProt P12643 or variants, homologs, or isoforms having substantial identity thereto. In embodiments, the gene encoding BMPis substantially identical to the nucleic acid sequence set forth in RefSeq (mRNA) NM_001200, or a variant or homolog having substantial identity thereto. In embodiments, the gnene encoding BMP is substantially identical to the nucleic acid sequence set forth in Ensembl reference number ENSG00000125845, or variants, homologs, or isoforms having substantial identity thereto. In embodiments, the amino acid sequence or nucleic acid sequence is the sequence known at the time of filing of the present application.

The term “EGF” as referred to herein includes any of the recombinant or naturally-occurring forms of epidermal growth factor (EGF) protein or variants or homologs thereof that maintain EGF activity (e.g. within at least 50%, 80%, 90%, 95%, 96%, 97%, 98%, 99% or 100% activity compared to EGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGF protein. In embodiments, the EGF protein is substantially identical to the protein identified by the UniProt reference number P01133 or a variant or homolog having substantial identity thereto. In embodiments, the EGF protein is substantially identical (e.g., at least 85%, 90%, 95%, 96%, 97%, 98%, 99% or 100% identical) to the amino acid sequence: NSDSECPLSHDGYCLHDGVCMYIEALDKYACNCVVGYIGERCQYRDLKWWELR. In embodiments, EGF is a human EGF protein. In embodiments, EGF is the protein identified by the NCBI sequence reference NP_001171601.1, or an isoform or naturally occurring mutant or variant thereof. In various embodiments, EGF is the protein as identified by the NCBI sequence reference NP_001171602.1, or an isoform or naturally occurring mutant or variant thereof. In various embodiments, EGF is the protein as identified by the NCBI sequence reference NP_001954.2, or an isoform or naturally occurring mutant or variant thereof. In certain embodiments, EGF is the protein as identified by the NCBI sequence reference NP_001343950.1, or an isoform or naturally occurring mutant or variant thereof.

A “Wnt protein” as referred to herein includes any of the recombinant or naturally-occurring forms of the wingless-type MMTV integration site family members (e.g., Wnt5a) or variants or homologs thereof that maintain Wnt activity (e.g. within at least 50%, 80%, 90%, 95%, 96%, 97%, 98%, 99% or 100% activity compared to Wnt). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Wnt protein. In embodiments, the Wnt protein is substantially identical to the protein identified by the UniProt reference number P41221 or a variant or homolog having substantial identity thereto. In embodiments, the Wnt protein is substantially identical to the protein identified by the UniProt reference number P22725 or a variant or homolog having substantial identity thereto.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “primordial germ cell” or “PGC” as provided herein refers to a cell established early in mammalian development, originating in the proximal region of the epiblast, close to the extraembryonic endoderm. A PGS is the earliest recognizable precursors of gametes, arise outside the gonads and migrate into the gonads during early embryonic development. In embodiments, the PGC is a Homo sapiens PGC, Pan troglodytes PGC, Pan paniscus PGC, Macaca mulatta PGC, Macaca fascicularis PGC, Pongo pygmaeus PGC, Gorilla beringer PGC, Gorilla beringer graueri PGC, Gorilla beringer beringei PGC, Gorilla gorilla PGC, Gorilla gorilla gorilla PGC, or Gorilla gorilla diehli PGC. In embodiments, the PGC is a Homo sapiens PGC. In embodiments, the PGC is a Pan troglodytes PGC. In embodiments, the PGC is a Pan paniscus PGC. In embodiments, the PGC is a Macaca mulatta PGC. In embodiments, the PGC is aMacaca fascicularis PGC. In embodiments, the PGC is a Pongo pygmaeus PGC. In embodiments, the PGC is a Gorilla beringer PGC. In embodiments, the PGC is a Gorilla beringer graueri PGC. In embodiments, the PGC is a Gorilla beringer beringei PGC. In embodiments, the PGC is a Gorilla gorilla PGC. In embodiments, the PGC is a Gorilla gorilla gorilla PGC. In embodiments, the PGC is a Gorilla gorilla diehli PGC. Any of the methods provided herein are contemplated to be used to generate any mammalian PGC (e.g., human, primate). In embodiments, the PGC is CXXR4+/PDGFRα⁻ and GARP⁻. In embodiments, the PGC differentiates into an oocyte or a sperm cell. In embodiments, the PGC has the ability to form an oocyte or a sperm cell. In embodiments, the PGC expresses any one of the surface expression markers set forth in Table 5 and indicated as PGC marker. “Expressing” and “expression” in the context of cells and genes or proteins (e.g., cell surface markers) is used according to its costumary meaning in the biological arts and refers to the ability of a cell to synthesize a protein or polypeptide (e.g., surface marker) and the protein is then detectable on the surface of the cell or intracellularly. Methods of detecting expression of intracellularly expressed porteins or surface marker proteins in vitro or in vivo are well known in the art and include without limitation immunofluorescence, fluorescence activated cell sorting.

The term “epiblast” or “primitive ectoderm” is used herein according to its customary meaning in the art and refers to one of two distinct layers arising from the inner cell mass in the mammalian blastocyst. The epiblast forms the embryo through its differentiation into the three primary germ layers, ectoderm, mesoderm and endoderm, during gastrulation. The amnionic ectoderm and extraembryonic mesoderm also originate from the epiblast.

The term “posterior epiblast” refers to the portion of the epiblast forming the posterior region thereof. Therefore, the posterior epiblast is a group of cells located at the posterior region if the epiblast. A “posterior epiblast cell” is a cell forming part or the posterior epiblast and/or expressing the same genes and/or proteins as a cell that form part of the posterior epiblast. In embodiments, the posterior epiblast cell expresses any of one of the proteins selected from Nodal, Comes, GSC, Lin28a, Fgf8, Sox2, Pouf5f1/Oct4, Nanog, Fst, Brachyury/T, Mixl1, Sp5Id1, Lefty 2, Foxhl and Fgf19. In embodiments, the posterior epiblast cell expresses Nodal, Comes, GSC, Lin28a, Fgf8, Sox2, Pouf5f1/Oct4, Nanog, Fst, Brachyury/T, Mixl1, Sp5Id1, Lefty 2, Foxhl or Fgf19. Likewise, a “posterior epiblast population” refers to a plurality of cells including one or more posterior epiblast cells.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC) can be distinguished. Embryonic stem cells reside in the blastocyst and give rise to embryonic tissues, whereas somatic stem cells reside in adult tissues for the purpose of tissue regeneration and repair. In embodiments, the stem cell is a Homo sapiens stem cell, Pan troglodytes stem cell, Pan paniscus stem cell, Macaca mulatta stem cell, Macaca fascicularis stem cell, Pongo pygmaeus stem cell, Gorilla beringer stem cell, Gorilla beringer graueri stem cell, Gorilla beringer beringei stem cell, Gorilla gorilla stem cell, Gorilla gorilla gorilla stem cell, or Gorilla gorilla diehli stem cell. In embodiments, the stem cell is a Homo sapiens stem cell. In embodiments, the stem cell is a Pan troglodytes stem cell. In embodiments, the stem cell is a Pan paniscus stem cell. In embodiments, the stem cell is a Macaca mulatta stem cell. In embodiments, the stem cell is a Macaca fascicularis stem cell. In embodiments, the stem cell is a Pongo pygmaeus stem cell. In embodiments, the stem cell is a Gorilla beringer stem cell. In embodiments, the stem cell is a Gorilla beringer graueri stem cell. In embodiments, the stem cell is a Gorilla beringer beringei stem cell. In embodiments, the stem cell is a Gorilla gorilla stem cell. In embodiments, the stem cell is a Gorilla gorilla gorilla stem cell. In embodiments, the stem cell is a Gorilla gorilla diehli stem cell.

As used herein, “pluripotent stem cells” refers to cells that may be derived from any source and that are capable, under appropriate conditions, of producing progeny of different cell types that are derivatives of all of the 3 germinal layers (endoderm, mesoderm, and ectoderm). Pluripotent stem cells cells may have the ability to form a teratoma in 812 week old SOD mice and/or the ability to form identifiable cells of all three germ layers in tissue culture. Included in the definition of pluripotent stem cells are embryonic cells of various types including human embryonic stem cells, (see, e.g., Thomson et al. (1998) Science 282: 1145) and human embryonic germ cells (see, e.g., Shatublott et al., (1998) Pr•oc. Natl. Acad. Sea. USA 95:13726,), stem cells created by nuclear transfer technology (U.S. Pat. Application Publication No. 2002/0046410), as well as induced pluripotent stem cells (see, e.g. Yu et al., (2007) Science 318:5858); Takahashi. et al., (2007) Cell131(5):861). The pluripotent stem cells may be established as cell lines, thus providing a continual source of pluripotent stem cells. It is contemplated that any of the embodiments of the invention described herein may be practiced by substituting one or more of the following sub-groupings of pluripotent stem cells for pluripotent stem cells: human embryonic stem cells, human embryonic germ cells, rhesus stem cells, marmoset stem cells, nuclear transfer stem cells and/or induced pluripotent stem cells.

A “cell culture media” as provided herein can be any cell culture or tissue culture media appropriate or suitable for each sample, e.g., the cell types present in the pluripotent stem cell population, the posterior epiblast cell population or the cell population comprising a PGC. In embodiments, the pluripotent stem cell population is obtained from a mammalian subject, and the culture medium is appropriate for or suitable for culture of mammalian cells or tissues. Exemplary culture media include Eagle’s Minimum Essential Medium (EMEM); Dulbecco’s Modified Eagle’s Medium (DMEM); RPMI-1640; Ham’s Nutrient Mixtures; Iscove’s Modified Dulbecco’s Medium (IMDM); DMEM/F12; Ham’s F-12; Neuralbasal medium; McCoy’s 5A medium; Endothelial cell growth medium-2 (EGM-2) medium; Medium 199; MethoCult medium; Leibovitz’s L-15 Medium; IMDM; M2 medium; MCDB 131 medium; Skeletal muscle cell differentiation medium; ESF 921 growth medium; Epilife medium supplement; Express Five SF Medium; Lymphocytes Separation Medium; keratinocyte growth medium; M16 medium; M9 minimal medium; Mammary Epithelial Growth Medium; Terrific Broth medium; Endothelial cell growth medium-2 (EGM-2); ITS liquid medium supplement; AIMV medium; Barbour-Stoenner-Kelly-H (BSK-H) medium; BBL Brewers modified thioglycollate medium; BGJ medium; BioWhittaker Ultraculture medium; BMGY medium; bronchial epithelial cell growth medium; Broth Heart Infusion medium; Cellgro lymphocyte separation medium; CnT07 medium; EPC medium; ESGRO Complete PLUS Clonal Grade Medium; Ex-CELL 400 medium; explant medium; HBSS medium; HCMTM hepatocyte culture medium; Hibernate E medium; Histopaque density medium; Hybridoma SFM medium; IPL-41 medium; LHC-8 medium; LHC-9 medium; Linsmaier and Skoog (LS) medium; M17 medium; M3:10 medium; MCDB153 medium; Medium 200; Mesenchymal Stem Cell expansion medium; MesenPro medium; methionine/cysteine free tissue culture medium; MSC culture medium; N1 growth medium supplement; PrEC medium; renal Epithelial Basal Medium; rich defined medium; S2 medium; Sabouraud dextrose medium; serum-free free-style medium; serum-free medium containing Neurobasal-A Medium; serum-free N2.1 medium; SFM4CHO medium; skeletal muscle growth medium; StemSpan SFEM medium; thioglycollate medium; T-lymphocyte-conditioned medium; VP-SFM medium; YNB medium; adipocyte differentiation medium; chondrocyte differentiation medium; EGM-2 medium; M3434 methylcellulose-based medium; Mesencult medium and Murashige and Skoog medium.

In embodiments, the culture medium or growth medium can contain supplements or additional factors, such as any one or more of supplements for nutrients, amino acids, vitamins, salts, serum proteins, carbohydrates, cofactors, trace elements, media supplements such as GlutaMax™ and antibiotics. In embodiments, the culture medium or growth medium contains serum. In some embodiments, the serum is fetal bovine serum, human serum, serum derived from human plasma or whole blood, human whole blood, serum or plasma or patient derived whole blood, serum or plasma. In embodiments, the serum is human serum, plasma or whole blood, or patient derived serum, plasma, or whole blood. In some embodiments, the serum is fetal bovine serum (FBS). In embodiments, the culture medium or growth medium contains serum at a concentration of at or about 1%, 2%, 3%, 4%, 5%, 6%, 7%, 8%, 9%, 10%, 11%, 12%, 13%, 14%, or 15%, or within a range defined by any of the foregoing values. In embodiments, the culture medium or growth medium is serum-free medium. In embodiments, the culture medium or growth medium is serum-free medium, with or without growth factor supplements. In embodiments, the culture medium or growth medium contains a media supplement or growth supplement such as GlutaMax™. In embodiments, the culture medium or growth medium contains a combined supplement, such as an insulin, transferrin and selenium (ITS) supplement. In embodiments, the culture medium or growth medium contains an antibiotic. Other exemplary supplements include EDTA; HEPES; L-glutamine; LPS; MEM; XAV939; amphotericin B; ampicillin; bovine serum albumin; bovine serum; chicken serum; collagenase; dithiothreitol; fetal bovine serum; gelatin; gentamicin; glucose; horse serum; hydrocortisone; hygromycin B; ionomycin; monensin; penicillin; poly-L-lysine; puromycin; rapamycin; streptomycin; trypsin; and wortmannin. In embodiments, the culture medium is RPMI containing 2 mM GlutaMax™, 1 × insulin, transferrin and selenium (ITS), antibiotics and with or without 10% FBS. In embodiments, the culture medium includes non-naturally occurring components, which are components that do not naturally occur in a human or primate organism (e.g., antibiotics, bovine serums, small molecules, such as antagonists/inhibitors or agonists provided herein).

In embodiments, the cell population (e.g., pluripotent stem cell population, posterior epiblast cell population, cell population comprising a PGC) is in a cell culture container. A “cell culture container” as provided herein refers to any container suitable for the in vitro propagation of cells (e.g., pluripotent stem cells, posterior epiblast cells, cell populations including a PGC). In embodiments, the cell culture container is a cell culture plate or a tissue culture plate or a bottle. In embodiments, the cell culture container is any known cell or tissue culture container. In embodiments, the cell culture container is a multiwell cell culture plates, such as a 6-, 12-, 24-, 48- or 96-well plates.

In embodiments, the culturing is carried out in a vessel or container comprising a surface coating. In embodiments, the culturing is carried out in the presence of a support that includes a surface coating. Exemplary materials for surface coating include collagen, gelatin, elastin, fibrin, fibronectin, laminin, and fibroin, among others; polysaccharide-based glycosaminoglycans such as chondroitin sulfate; hyaluronic acid and its derivatives; chitosan; alginate; cellulose and its derivatives, such as methylcellulose; hydroxypropylcellulose and carboximethylcellulose; dextran; agar; agarose; starch; carrageenan; thermally gelling polysaccharides such as kappa-carrageenan and iota-carrageenangalactomannan such as guar gum; xanthan or xanthan gum; extracellular matrix;pullulan; PEG hydrogels or peptide-based hydrogels. Examples of commercially available gels include Life Technologies, AlgiMatrix, BD MATRIGEL™, Glycosan HyStem, Extracel, BD PuraMatrix, Glycosan PEGgel, QGel SA and QGel. In some aspects, MATRIGEL™ includes a biological product typically comprising Laminin, Collagen IV, Entactin, and heparin sulfate proteoglycan among other constituents. In some embodiments, the surface coating comprises MATRIGEL™, collagen, fibronectin, extracellular matrix, PEG hydrogels or peptide-based hydrogels.

In embodiments, the culturing is carried out in a cell culture incubator. In embodiments, conditions for cell culture include conditions typically used for cell culture or tissue culture, including any particular conditions used for culture of a particular cell type or tissue type.

In embodiments, the culturing is carried out under a constant CO₂ level. In embodiments, the constant CO₂ level is approximately 5% CO₂. In embodiments, the culturing is carried out at a constant temperature. In embodiments, the constant temperature is at or about 30° C., 31° C., 32° C., 33° C., 34° C., 35° C., 36° C., 37° C., 38° C., 39° C., or 40° C. In embodiments, the constant temperature is at or about 37° C. In embodiments, the culturing is carried out under a constant O₂ level. In embodiments, the constant O₂ level is approximately 20% O₂ or atmospheric O₂ level.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a cell as provided herein and an agonist, inhibitor or growth factor (e.g., Wnt agonist, BMP, SCF, EGF, Wnt inhbitor).

As defined herein, the term “inhibition”, “inhibit”, “inhibiting,” “repression,” repressing,” “silencing,” “silence” and the like when used in reference to a composition as provided herein (e.g., Wnt inhibitor) refer to negatively affecting (e.g., decreasing) the activity (e.g., transcription) of a nucleic acid sequence (e.g., decreasing transcription of a gene) relative to the activity of the nuclei acid sequence (e.g., transcription of a gene) in the absence of the composition (e.g., Wnt inhibitor). In embodiments, inhibition refers to reduction of a disease or symptoms of disease. Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In embodiments, the inhibition is 1.5-fold, 2-fold, 3-fold, 4-fold, 5-fold, 10-fold, or more in comparison to a control.

An “inhibitor” refers to a compound (e.g. Wnt inhibitor) that reduces activity when compared to a control, such as absence of the compound or a compound with known inactivity. An “inhbitor” or “antagonist” refers to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist. An “inhibitor” (e.g., Wnt inhibitor) as provided herein may be a compound or small molecule that inhibits a signaling pathway e.g., by binding, partially or totally blocking stimulation of said signaling pathway (e.g., Wnt signaling pathway), decrease, prevent, or delay activation of said signaling pathway (e.g., Wnt signaling pathway), or inactivate, desensitize, or down-regulate signal transduction, gene expression or enzymatic activity of a signaling pathway (e.g., Wnt signaling pathway). In embodiments, the Wnt inhibitor inhibits Wnt activity or expression. In embodiments, the Wnt inhibitor is a compound or a small molecule. In embodiments, the Wnt inhibitor is an antibody.

An “agonist” refers to a compound (e.g. compounds described herein) that increases activity when compared to a control, such as absence of the compound or a compound with known inactivity. The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist. An “agonist” (e.g., Wnt agonist, TGFβ agonist) as provided herein may be a compound or small molecule that activates a signaling pathway e.g., by binding, partially or totally stimulating said signaling pathway (e.g., Wnt signaling pathway, TGFβ signaling pathway), increase, enhance, or accelerate activation of said signaling pathway (e.g., Wnt signaling pathway, TGFβ signaling pathway), or activate, sensitize, or up-regulate signal transduction, gene expression or enzymatic activity of a signaling pathway (e.g., Wnt signaling pathway, TGFβ signaling pathway). In embodiments, the Wnt agonist activates Wnt activity or expression. In embodiments, the Wnt agonist is a compound or a small molecule. In embodiments, the Wnt agnonist is an antibody. In embodiments, the TGFβ agonist activates TGFβ activity or expression. In embodiments, the TGFβ agonist is a compound or a small molecule. In embodiments, the TGFβ agnonist is an antibody.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof’ refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease or condition is infertility.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing reoccurring of the disease and/or relieving the disease, that is, causing the regression of clinical symptoms by administration of a protective composition after their initial appearance. For example, certain methods herein treat infertility. For example, certain methods herein treat infertility by administering the PGCs provided herein including embodiments thereof to a subject, wherein the subject is infertile.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination. For the methods of treating infertility provided herein including embodiments thereof, the term refers to ameliorating the condition of infertility. For example, by administering a PGC as provided herein to an infertile subject, said subject may be able to produce oocytes or sperms derived from said PGC and therefore become fertile.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example infertility therapies such as. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, poly glycolic acids and copolymers (such as latex functionalized sepharose(TM), agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing infertility for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like., that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Methods

The methods and compositions provided herein provide for a simplified 2D in vitro platform to generate human PGCs within, for example, as little as 3.5 days, of hPSC differentiation. The methods and compositions provided herein including embodiments thereof, may be used, inter alia, to simplify the production of human PGCs for drug discovery, disease modeling and infertility treatments. Furthermore, the methods and compositions provided herein including embodiments thereof, may be used, inter alia, as research tool to investigate key lineage intermediates and extracellular signals in PGC specification.

In an aspect is provided a method of forming a primordial germ cell (PGC) in vitro, the method including: (i) contacting a pluripotent stem cell population with a wingless integrated (WNT) agonist and a transforming growth factor beta (TGFβ) agonist, thereby forming a posterior epiblast cell population; (ii) contacting the posterior epiblast cell population with a WNT inhibitor and removing the WNT agonist and the TGFβ agonist, thereby forming a PGC.

In an aspect a method of isolating a primordial germ cell (PGC) is provided, the method including: (i) contacting a pluripotent stem cell population with a WNT agonist and a TGFβ agonist in vitro, thereby forming a posterior epiblast cell population; (ii) contacting the posterior epiblast cell population with a WNT inhibitor and removing the WNT agonist and the TGFβ agonist; thereby forming a cell population including a PGC; and (iii) separating a CXCR4^{+/}PDGFRα⁻/GARP cell from the cell population including a PGC, thereby isolating the PGC.

In an aspect a method of forming a primordial germ cell (PGC) in vitro is provided, The method includes (i) contacting a pluripotent stem cell population with a wingless integrated (WNT) agonist and a transforming growth factor beta (TGFβ) agonist, thereby forming a posterior epiblast cell population; (ii) contacting the posterior epiblast cell population with a WNT inhibitor, wherein prior to the contacting of step (ii) the WNT agonist and the TGFβ agonist are removed, thereby forming a PGC.

In an aspect a method of isolating a primordial germ cell (PGC) is provided. The method includes, (i) contacting a pluripotent stem cell population with a WNT agonist and a TGFβ agonist in vitro, thereby forming a posterior epiblast cell population; (ii) contacting the posterior epiblast cell population with a WNT inhibitor, wherein prior to the contacting of step (ii) the WNT agonist and the TGFβ agonist are removed, thereby forming a cell population comprising a PGC; and (iii) separating a CXCR4^{+/}PDGFRα⁻/GARP cell from the cell population comprising a PGC, thereby isolating said PGC.

The term “separating” as provided herein is used according to its costumary meaning in the biological art and refers to the physical separation of one or more cells from a cell population (plurality of cells) in a cell culture container.

A “pluripotent stem cell population” is a cell population including one or more pluripotent stem cells. In embodiments, at least 10%, 20%, 30%, 40%, 50%, 60%, 70%, 80%, 90%, 95%, 98%, or 99% of all cells of a pluripotent stem cell population are pluripotent stem cells. In embodiments, about 10%, 20%, 30%, 40%, 50%, 60%, 70%, 80%, 90%, 95%, 98%, or 99% of all cells of a pluripotent stem cell population are pluripotent stem cells. In embodiments, 10%, 20%, 30%, 40%, 50%, 60%, 70%, 80%, 90%, 95%, 98%, or 99% of all cells of a pluripotent stem cell population are pluripotent stem cells. In embodiments, between 10%-95%, of all cells of a pluripotent stem cell population are pluripotent stem cells. In embodiments, between 15%-95%, of all cells of a pluripotent stem cell population are pluripotent stem cells. In embodiments, between 20%-95%, of all cells of a pluripotent stem cell population are pluripotent stem cells. In embodiments, between 25%-95%, of all cells of a pluripotent stem cell population are pluripotent stem cells. In embodiments, between 30%-95%, of all cells of a pluripotent stem cell population are pluripotent stem cells. In embodiments, between 35%-95%, of all cells of a pluripotent stem cell population are pluripotent stem cells. In embodiments, between 40%-95%, of all cells of a pluripotent stem cell population are pluripotent stem cells. In embodiments, between 45%-95%, of all cells of a pluripotent stem cell population are pluripotent stem cells. In embodiments, between 50%-95%, of all cells of a pluripotent stem cell population are pluripotent stem cells. In embodiments, between 55%-95%, of all cells of a pluripotent stem cell population are pluripotent stem cells. In embodiments, between 60%-95%, of all cells of a pluripotent stem cell population are pluripotent stem cells. In embodiments, between65%-95%, of all cells of a pluripotent stem cell population are pluripotent stem cells. In embodiments, between 70%-95%, of all cells of a pluripotent stem cell population are pluripotent stem cells. In embodiments, between 75%-95%, of all cells of a pluripotent stem cell population are pluripotent stem cells. In embodiments, between 80%-95%, of all cells of a pluripotent stem cell population are pluripotent stem cells. In embodiments, between 85%-95%, of all cells of a pluripotent stem cell population are pluripotent stem cells. In embodiments, between 90%-95%, of all cells of a pluripotent stem cell population are pluripotent stem cells. In embodiments, 100% of all cells of a pluripotent stem cell population are pluripotent stem cells. In embodiments, the pluripotent stem cell expresses any one of the surface expression markers set forth in Table 5 and indicated as pluripotent stem cell marker.

A “posterior epiblast cell population” is a cell population including one or more posterior epiblast cells. In embodiments, at least 10%, 20%, 30%, 40%, 50%, 60%, 70%, 80%, 90%, 95%, 98%, or 99% of all cells of a posterior epiblast cell population are posterior epiblast cells. In embodiments, about 10%, 20%, 30%, 40%, 50%, 60%, 70%, 80%, 90%, 95%, 98%, or 99% of all cells of a posterior epiblast cell population are posterior epiblast cells. In embodiments, 10%, 20%, 30%, 40%, 50%, 60%, 70%, 80%, 90%, 95%, 98%, or 99% of all cells of a posterior epiblast cell population are posterior epiblast cells. In embodiments, between 10%-95%, of all cells of a posterior epiblast cell population are posterior epiblast cells. In embodiments, between 15%-95%, of all cells of a posterior epiblast cell population are posterior epiblast cells. In embodiments, between 20%-95%, of all cells of a posterior epiblast cell population are posterior epiblast cells. In embodiments, between 25%-95%, of all cells of a posterior epiblast cell population are posterior epiblast cells. In embodiments, between 30%-95%, of all cells of a posterior epiblast cell population are posterior epiblast cells. In embodiments, between 35%-95%, of all cells of a posterior epiblast cell population are posterior epiblast cells. In embodiments, between 40%-95%, of all cells of a posterior epiblast cell population are posterior epiblast cells. In embodiments, between 45%-95%, of all cells of a posterior epiblast cell population are posterior epiblast cells. In embodiments, between 50%-95%, of all cells of a posterior epiblast cell population are posterior epiblast cells. In embodiments, between 55%-95%, of all cells of a posterior epiblast cell population are posterior epiblast cells. In embodiments, between 60%-95%, of all cells of a posterior epiblast cell population are posterior epiblast cells. In embodiments, between 65%-95%, of all cells of a posterior epiblast cell population are posterior epiblast cells. In embodiments, between 70%-95%, of all cells of a posterior epiblast cell population are posterior epiblast cells. In embodiments, between 75%-95%, of all cells of a posterior epiblast cell population are posterior epiblast cells. In embodiments, between 80%-95%, of all cells of a posterior epiblast cell population are posterior epiblast cells. In embodiments, between 85%-95%, of all cells of a posterior epiblast cell population are posterior epiblast cells. In embodiments, between 90%-95%, of all cells of a posterior epiblast cell population are posterior epiblast cells. In embodiments, 100% of all cells of a posterior epiblast cell population are posterior epiblast cells. In embodiments, the posterior epiblast cell expresses any of one of the proteins selected from Nodal, Comes, GSC, Lin28a, Fgf8, Sox2, Pouf5f1/Oct4, Nanog, Fst, Brachyury/T, Mixl1, Sp5Id1, Lefty 2, Foxhl and Fgf19. In embodiments, the posterior epiblast cell expresses Nodal, Comes, GSC, Lin28a, Fgf8, Sox2, Pouf5f1/Oct4, Nanog, Fst, Brachyury/T, Mixl1, Sp5Id1, Lefty 2, Foxhl or Fgf19. In embodiments, the posterior epiblast cell expresses Nodal, Comes, GSC, Lin28a, Fgf8, Sox2, Pouf5f1/Oct4, Nanog, Fst, Brachyury/T, Mixl1, Sp5Id1, Lefty 2, Foxhl and Fgf19.

For the methods provided herein the pluripotent stem cell population is contacted with a WNT agonist and a TGFβ agonist to form a posterior epiblast cell population. After the posterior epiblast cell population is formed the WNT agonist and the TGFβ agonist are removed and subsequently the posterior epiblast cell population is contacted with a WNT inhibitor. Therefore, prior to the contacting of the posterior epiblast cell population with a WNT inhibitor in step (ii) the WNT agonist and the TGFβ agonist are removed. A cell population as provided herein wherein an agonist has been removed from refers to a cell population in a cell culture medium that does not include detectable or otherwise activating forms of said agonist.

“WNT inhibitor” refers to a compound that reduces activity when compared to a control, such as absence of the compound or a compound with known inactivity. An “WNT inhbitor” or “WNT antagonist” refers to a substance capable of detectably decreasing the expression or activity of a given gene or protein of the WNT signaling pathway and its components. The WNT antagonist can decrease expression or activity 10%, 20%, 30%, 40%, 50%, 60%, 70%, 80%, 90% or more in comparison to a control in the absence of the WNT antagonist. In certain instances, expression or activity is 1.5-fold, 2-fold, 3-fold, 4-fold, 5-fold, 10-fold or lower than the expression or activity in the absence of the WNT antagonist. A WNT inhibitor as provided herein may be a compound or small molecule that inhibits the WNT signaling pathway e.g., by binding, partially or totally blocking stimulation of said signaling pathway, decrease, prevent, or delay activation of said signaling pathway, or inactivate, desensitize, or down-regulate signal transduction, gene expression or enzymatic activity of the WNT signaling pathway. In embodiments, the Wnt inhibitor inhibits Wnt activity or WNT expression. In embodiments, the Wnt inhibitor is a compound or a small molecule. In embodiments, the Wnt inhibitor is an antibody.

A “WNT agonist” refers to a compound (e.g. compounds described herein) that increases activity when compared to a control, such as absence of the compound or a compound with known inactivity. The term “WNT agonist,” “WNT activator,” “WNT upregulator,” etc. refer to a substance capable of detectably increasing the expression or activity of the WNT signaling pathway and its components. The WNT agonist can increase expression or activity 10%, 20%, 30%, 40%, 50%, 60%, 70%, 80%, 90% or more in comparison to a control in the absence of the WNT agonist. In certain instances, expression or activity is 1.5-fold, 2-fold, 3-fold, 4-fold, 5-fold, 10-fold or higher than the expression or activity in the absence of the WNT agonist. A “WNT agonist” as provided herein may be a compound or small molecule that activates teh WNT signaling pathway e.g., by binding, partially or totally stimulating said signaling pathway, increase, enhance, or accelerate activation of said signaling pathway, or activate, sensitize, or up-regulate signal transduction, gene expression or enzymatic activity of a signaling pathway. In embodiments, the WNT agonist activates WNT activity or expression. In embodiments, the WNT agonist is a compound or a small molecule. In embodiments, the WNT agnonist is an antibody.

A “TGFβ agonist” refers to a compound (e.g. compounds described herein) that increases activity when compared to a control, such as absence of the compound or a compound with known inactivity. The term “TGFβ agonist,” “TGFβ activator,” “TGFβ upregulator,” etc. refer to a substance capable of detectably increasing the expression or activity of the TGFβ signaling pathway and its components. The TGFβ agonist can increase expression or activity 10%, 20%, 30%, 40%, 50%, 60%, 70%, 80%, 90% or more in comparison to a control in the absence of the TGFβ agonist. In certain instances, expression or activity is 1.5-fold, 2-fold, 3-fold, 4-fold, 5-fold, 10-fold or higher than the expression or activity in the absence of the TGFβ agonist. A “TGFβ agonist” as provided herein may be a compound or small molecule that activates teh TGFβ signaling pathway e.g., by binding, partially or totally stimulating said signaling pathway, increase, enhance, or accelerate activation of said signaling pathway, or activate, sensitize, or up-regulate signal transduction, gene expression or enzymatic activity of a signaling pathway. In embodiments, the TGFβ agonist activates TGFβ activity or expression. In embodiments, the TGFβ agonist is a compound or a small molecule. In embodiments, the TGFβ agnonist is an antibody.

In embodiments, the WNT agonist is CHIR99021. In embodiments, the WNT agonist is lithium chloride. In embodiments, the WNT agonist is SB216763. In embodiments, the WNT agonist is BIO. In embodiments, the WNT agonist is WNT3A.

In embodiments, the TGFβ agonist is activin. In embodiments, the TGFβ agonist is IDE1. In embodiments, the TGFβ agonist is Nodal. In embodiments, the TGFβ agonist is TGFbeta-1. In embodiments, the TGFβ agonist is GDF11.

In embodiments, the WNT inhibitor is XAV939. In embodiments, the WNT inhibitor is iCRT3. In embodiments, the WNT inhibitor is iCRT14. In embodiments, the WNT inhibitor is LF3. In embodiments, the WNT inhibitor is IWR-1-endo. In embodiments, the WNT inhibitor is JW67. In embodiments, the WNT inhibitor is JW74.

In embodiments, the TGFβ agonist is activin. The term “Activin-A protein” or “Activin-A” as used herein includes any of the recombinant or naturally-occurring forms of Activin-A, or variants or homologs thereof that maintain Activin-A activity (e.g. within at least 50%, 80%, 90%, 95%, 96%, 97%, 98%, 99% or 100% activity compared to Activin-A). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Activin-A protein. In embodiments, the Activin-A protein is substantially identical to the protein identified by the UniProt reference number P08476 or a variant or homolog having substantial identity thereto.

In embodiments, the pluripotent stem cell population is contacted with 10 ng to 120 ng Activin-A. In embodiments, the pluripotent stem cell population is contacted with 15 ng to 120 ng Activin-A. In embodiments, the pluripotent stem cell population is contacted with 20 ng to 120 ng Activin-A. In embodiments, the pluripotent stem cell population is contacted with 25 ng to 120 ng Activin-A. In embodiments, the pluripotent stem cell population is contacted with 30 ng to 120 ng Activin-A. In embodiments, the pluripotent stem cell population is contacted with 35 ng to 120 ng Activin-A. In embodiments, the pluripotent stem cell population is contacted with 40 ng to 120 ng Activin-A. In embodiments, the pluripotent stem cell population is contacted with 45 ng to 120 ng Activin-A. In embodiments, the pluripotent stem cell population is contacted with 50 ng to 120 ng Activin-A. In embodiments, the pluripotent stem cell population is contacted with 55 ng to 120 ng Activin-A. In embodiments, the pluripotent stem cell population is contacted with 60 ng to 120 ng Activin-A. In embodiments, the pluripotent stem cell population is contacted with 65 ng to 120 ng Activin-A. In embodiments, the pluripotent stem cell population is contacted with 70 ng to 120 ng Activin-A. In embodiments, the pluripotent stem cell population is contacted with 75 ng to 120 ng Activin-A. In embodiments, the pluripotent stem cell population is contacted with 80 ng to 120 ng Activin-A. In embodiments, the pluripotent stem cell population is contacted with 85 ng to 120 ng Activin-A. In embodiments, the pluripotent stem cell population is contacted with 90 ng to 120 ng Activin-A. In embodiments, the pluripotent stem cell population is contacted with 95 ng to 120 ng Activin-A. In embodiments, the pluripotent stem cell population is contacted with 100 ng to 120 ng Activin-A. In embodiments, the pluripotent stem cell population is contacted with 105 ng to 120 ng Activin-A. In embodiments, the pluripotent stem cell population is contacted with 110 ng to 120 ng Activin-A. In embodiments, the pluripotent stem cell population is contacted with 115 ng to 120 ng Activin-A.

In embodiments, the pluripotent stem cell population is contacted with 10 ng to 115 ng Activin-A. In embodiments, the pluripotent stem cell population is contacted with 10 ng to 110 ng Activin-A. In embodiments, the pluripotent stem cell population is contacted with 10 ng to 105 ng Activin-A. In embodiments, the pluripotent stem cell population is contacted with 10 ng to 100 ng Activin-A. In embodiments, the pluripotent stem cell population is contacted with 10 ng to 95 ng Activin-A. In embodiments, the pluripotent stem cell population is contacted with 10 ng to 90 ng Activin-A. In embodiments, the pluripotent stem cell population is contacted with 10 ng to 85 ng Activin-A. In embodiments, the pluripotent stem cell population is contacted with 10 ng to 80 ng Activin-A. In embodiments, the pluripotent stem cell population is contacted with 10 to 75 ng Activin-A. In embodiments, the pluripotent stem cell population is contacted with 10 ng to 70 ng Activin-A. In embodiments, the pluripotent stem cell population is contacted with 10 ng to 65 ng Activin-A. In embodiments, the pluripotent stem cell population is contacted with 10 ng to 60 ng Activin-A. In embodiments, the pluripotent stem cell population is contacted with 10 to 55 ng Activin-A. In embodiments, the pluripotent stem cell population is contacted with 10 ng to 50 ng Activin-A. In embodiments, the pluripotent stem cell population is contacted with 10 ng to 45 ng Activin-A. In embodiments, the pluripotent stem cell population is contacted with 10 ng to 40 ng Activin-A. In embodiments, the pluripotent stem cell population is contacted with 10 ng to 35 ng Activin-A. In embodiments, the pluripotent stem cell population is contacted with 10 ng to 30 ng Activin-A. In embodiments, the pluripotent stem cell population is contacted with 10 to 25 ng Activin-A. In embodiments, the pluripotent stem cell population is contacted with 10 ng to 20 ng Activin-A. In embodiments, the pluripotent stem cell population is contacted with 10 ng to 15 ng Activin-A.

In embodiments, the pluripotent stem cell population is contacted with about 10 ng, 15 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 105 ng, 110 ng, 115 ng or 120 ng Activin-A. In embodiments, the pluripotent stem cell population is contacted with 10 ng, 15 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 105 ng, 110 ng, 115 ng or 120 ng Activin-A. In embodiments, the pluripotent stem cell population is contacted with about 10 ng Activin-A. In embodiments, the pluripotent stem cell population is contacted with 10 ng Activin-A.

In embodiments, the WNT agonist is CHIR99021. “CHIR99021” in a customary sense refers to the compound identified by Cas Registry No.: 252917-06-9 or the chemical name 6-((2-((4-(2,4-Dichlorophenyl)-5-(4-methyl-1H-imidazol-2-yl)pyrimidin-2-yl)amino)ethyl)amino)nicotinonitrile.

In embodiments, the pluripotent stem cell population is contacted with 0.1 µM to 15 µM CHIR99021. In embodiments, the pluripotent stem cell population is contacted with 0.5 µM to 15 µM CHIR99021. In embodiments, the pluripotent stem cell population is contacted with 1 µM to 15 µM CHIR99021. In embodiments, the pluripotent stem cell population is contacted with 1.5 µM to 15 µM CHIR99021. In embodiments, the pluripotent stem cell population is contacted with 2 µM to 15 µM CHIR99021. In embodiments, the pluripotent stem cell population is contacted with 2.5 µM to 15 µM CHIR99021. In embodiments, the pluripotent stem cell population is contacted with 3 µM to 15 µM CHIR99021. In embodiments, the pluripotent stem cell population is contacted with 3.5 µM to 15 µM CHIR99021. In embodiments, the pluripotent stem cell population is contacted with 4 µM to 15 µM CHIR99021. In embodiments, the pluripotent stem cell population is contacted with 4.5 µM to 15 µM CHIR99021. In embodiments, the pluripotent stem cell population is contacted with 5 µM to 15 µM CHIR99021. In embodiments, the pluripotent stem cell population is contacted with 5.5 µM to 15 µM CHIR99021. In embodiments, the pluripotent stem cell population is contacted with 6 µM to 15 µM CHIR99021. In embodiments, the pluripotent stem cell population is contacted with 6.5 µM to 15 µM CHIR99021. In embodiments, the pluripotent stem cell population is contacted with 7 µM to 15 µM CHIR99021. In embodiments, the pluripotent stem cell population is contacted with 7.5 µM to 15 µM CHIR99021. In embodiments, the pluripotent stem cell population is contacted with 8 µM to 15 µM CHIR99021. In embodiments, the pluripotent stem cell population is contacted with 8.5 µM to 15 µM CHIR99021. In embodiments, the pluripotent stem cell population is contacted with 9 µM to 15 µM CHIR99021. In embodiments, the pluripotent stem cell population is contacted with 9.5 µM to 15 µM CHIR99021. In embodiments, the pluripotent stem cell population is contacted with 10 µM to 15 µM CHIR99021. In embodiments, the pluripotent stem cell population is contacted with 10.5 µM to 15 µM CHIR99021. In embodiments, the pluripotent stem cell population is contacted with 11 µM to 15 µM CHIR99021. In embodiments, the pluripotent stem cell population is contacted with 11.5 µM to 15 µM CHIR99021. In embodiments, the pluripotent stem cell population is contacted with 12 µM to 15 µM CHIR99021. In embodiments, the pluripotent stem cell population is contacted with 12.5 µM to 15 µM CHIR99021. In embodiments, the pluripotent stem cell population is contacted with 13 µM to 15 µM CHIR99021. In embodiments, the pluripotent stem cell population is contacted with 13.5 µM to 15 µM CHIR99021. In embodiments, the pluripotent stem cell population is contacted with 14 µM to 15 µM CHIR99021. In embodiments, the pluripotent stem cell population is contacted with 14.5 µM to 15 µM CHIR99021.

In embodiments, the pluripotent stem cell population is contacted with 0.1 µM to 14.5 µM CHIR99021. In embodiments, the pluripotent stem cell population is contacted with 0.1 µM to 14 µM CHIR99021. In embodiments, the pluripotent stem cell population is contacted with 0.1 µM to 13.5 µM CHIR99021. In embodiments, the pluripotent stem cell population is contacted with 0.1 µM to 13 µM CHIR99021. In embodiments, the pluripotent stem cell population is contacted with 0.1 µM to 12.5 µM CHIR99021. In embodiments, the pluripotent stem cell population is contacted with 0.1 µM to 12 µM CHIR99021. In embodiments, the pluripotent stem cell population is contacted with 0.1 µM to 11.5 µM CHIR99021. In embodiments, the pluripotent stem cell population is contacted with 0.1 µM to 11 µM CHIR99021. In embodiments, the pluripotent stem cell population is contacted with 0.1 µM to 10.5 µM CHIR99021. In embodiments, the pluripotent stem cell population is contacted with 0.1 µM to 10 µM CHIR99021. In embodiments, the pluripotent stem cell population is contacted with 0.1 µM to 9.5 µM CHIR99021. In embodiments, the pluripotent stem cell population is contacted with 0.1 µM to 9 µM CHIR99021. In embodiments, the pluripotent stem cell population is contacted with 0.1 µM to 8.5 µM CHIR99021. In embodiments, the pluripotent stem cell population is contacted with 0.1 µM to 8 µM CHIR99021. In embodiments, the pluripotent stem cell population is contacted with 0.1 µM to 7.5 µM CHIR99021. In embodiments, the pluripotent stem cell population is contacted with 0.1 µM to 7 µM CHIR99021. In embodiments, the pluripotent stem cell population is contacted with 0.1 µM to 6.5 µM CHIR99021. In embodiments, the pluripotent stem cell population is contacted with 0.1 µM to 6 µM CHIR99021. In embodiments, the pluripotent stem cell population is contacted with 0.1 µM to 5.5 µM CHIR99021. In embodiments, the pluripotent stem cell population is contacted with 0.1 µM to 5 µM CHIR99021. In embodiments, the pluripotent stem cell population is contacted with 0.1 µM to 4.5 µM CHIR99021. In embodiments, the pluripotent stem cell population is contacted with 0.1 µM to 4 µM CHIR99021. In embodiments, the pluripotent stem cell population is contacted with 0.1 µM to 3.5 µM CHIR99021. In embodiments, the pluripotent stem cell population is contacted with 0.1 µM to 3 µM CHIR99021. In embodiments, the pluripotent stem cell population is contacted with 0.1 µM to 2.5 µM CHIR99021. In embodiments, the pluripotent stem cell population is contacted with 0.1 µM to 2 µM CHIR99021. In embodiments, the pluripotent stem cell population is contacted with 0.1 µM to 1.5 µM CHIR99021. In embodiments, the pluripotent stem cell population is contacted with 0.1 µM to 1 µM CHIR99021. In embodiments, the pluripotent stem cell population is contacted with 0.1 µM to 0.5 µM CHIR99021.

In embodiments, the pluripotent stem cell population is contacted with about 0.1 µM, 0.5 µM, 1 µM, 1.5 µM, 2 µM, 2.5 µM, 3 µM, 3.5 µM, 4 µM, 4.5 µM, 5 µM, 5.5 µM, 6 µM, 6.5 µM, 7 µM, 7.5 µM, 8 µM, 8.5 µM, 9 µM, 9.5 µM, 10 µM, 10.5 µM, 11 µM, 11.5 µM, 12 µM, 12.5 µM, 13 µM, 13.5 µM, 14 µM, 14.5 µM or 15 µM CHIR99021. In embodiments, the pluripotent stem cell population is contacted with 0.1 µM, 0.5 µM, 1 µM, 1.5 µM, 2 µM, 2.5 µM, 3 µM, 3.5 µM, 4 µM, 4.5 µM, 5 µM, 5.5 µM, 6 µM, 6.5 µM, 7 µM, 7.5 µM, 8 µM, 8.5 µM, 9 µM, 9.5 µM, 10 µM, 10.5 µM, 11 µM, 11.5 µM, 12 µM, 12.5 µM, 13 µM, 13.5 µM, 14 µM, 14.5 µM or 15 µM CHIR99021. In embodiments, the pluripotent stem cell population is contacted with about 3 µM CHIR99021. In embodiments, the pluripotent stem cell population is contacted with 3 µM CHIR99021.

“Y-27632” in a customary sense refers to the compound identified by Cas Registry No.: 146986-50-7 or the chemical name (1R,4r)-4-((R)-I-aminoethyl)-N-(pyridin-4-yl)cyclohexanecarboxamide.

In embodiments, the pluripotent stem cell population is contacted with 5 µM to 50 µM Y-27632. In embodiments, the pluripotent stem cell population is contacted with 7.5 µM to 50 µM Y-27632. In embodiments, the pluripotent stem cell population is contacted with 10 µM to 50 µM Y-27632. In embodiments, the pluripotent stem cell population is contacted with 12.5 µM to 50 µM Y-27632. In embodiments, the pluripotent stem cell population is contacted with 15 µM to 50 µM Y-27632. In embodiments, the pluripotent stem cell population is contacted with 17.5 µM to 50 µM Y-27632.In embodiments, the pluripotent stem cell population is contacted with 20 µM to 50 µM Y-27632. In embodiments, the pluripotent stem cell population is contacted with 22.5 µM to 50 µM Y-27632.In embodiments, the pluripotent stem cell population is contacted with 25 µM to 50 µM Y-27632. In embodiments, the pluripotent stem cell population is contacted with 27.5 µM to 50 µM Y-27632. In embodiments, the pluripotent stem cell population is contacted with 30 µM to 50 µM Y-27632. In embodiments, the pluripotent stem cell population is contacted with 32.5 µM to 50 µM Y-27632. In embodiments, the pluripotent stem cell population is contacted with 35 µM to 50 µM Y-27632. In embodiments, the pluripotent stem cell population is contacted with 37.5 µM to 50 µM Y-27632. In embodiments, the pluripotent stem cell population is contacted with 40 µM to 50 µM Y-27632. In embodiments, the pluripotent stem cell population is contacted with 42.5 µM to 50 µM Y-27632. In embodiments, the pluripotent stem cell population is contacted with 45 µM to 50 µM Y-27632. In embodiments, the pluripotent stem cell population is contacted with 47.5 µM to 50 µM Y-27632.

In embodiments, the pluripotent stem cell population is contacted with 5 µM to 47.5 µM Y-27632. In embodiments, the pluripotent stem cell population is contacted with 5 µM to 45 µM Y-27632. In embodiments, the pluripotent stem cell population is contacted with 5 µM to 42.5 µM Y-27632. In embodiments, the pluripotent stem cell population is contacted with 5 µM to 40 µM Y-27632. In embodiments, the pluripotent stem cell population is contacted with 5 µM to 37.5 µM Y-27632. In embodiments, the pluripotent stem cell population is contacted with 5 µM to 35 µM Y-27632. In embodiments, the pluripotent stem cell population is contacted with 5 µM to 32.5 µM Y-27632. In embodiments, the pluripotent stem cell population is contacted with 5 µM to 30 µM Y-27632. In embodiments, the pluripotent stem cell population is contacted with 5 µM to 27.5 µM Y-27632. In embodiments, the pluripotent stem cell population is contacted with 5 µM to 25 µM Y-27632. In embodiments, the pluripotent stem cell population is contacted with 5 µM to 22.5 µM Y-27632. In embodiments, the pluripotent stem cell population is contacted with 5 µM to 20 µM Y-27632. In embodiments, the pluripotent stem cell population is contacted with 5 µM to 17.5 µM Y-27632. In embodiments, the pluripotent stem cell population is contacted with 5 µM to 15 µM Y-27632. In embodiments, the pluripotent stem cell population is contacted with 5 µM to 12.5 µM Y-27632. In embodiments, the pluripotent stem cell population is contacted with 5 µM to 10 µM Y-27632. In embodiments, the pluripotent stem cell population is contacted with 5 µM to 7.5 µM Y-27632.

In embodiments, the pluripotent stem cell population is contacted with about 5 µM, 7.5 µM, 10 µM, 12.5 µM, 15 µM, 17.5 µM, 20 µM, 22.5 µM, 25 µM, 27.5 µM, 30 µM, 32.5 µM, 35 µM, 37.5 µM, 40 µM, 42.5 µM, 45 µM, 47.5 µM or 50 µM Y-27632. In embodiments, the pluripotent stem cell population is contacted with 5 µM, 7.5 µM, 10 µM, 12.5 µM, 15 µM, 17.5 µM, 20 µM, 22.5 µM, 25 µM, 27.5 µM, 30 µM, 32.5 µM, 35 µM, 37.5 µM, 40 µM, 42.5 µM, 45 µM, 47.5 µM or 50 µM Y-27632. In embodiments, the pluripotent stem cell population is contacted with about 10 µM Y-27632. In embodiments, the pluripotent stem cell population is contacted with 10 µM Y-27632.

In embodiments, the contacting of step (i) is for the duration of about 12 hours or less. In embodiments, the contacting of step (i) is for the duration of about 0.5 hours to about 12 hours. In embodiments, the contacting of step (i) is for the duration of about 1 hours to about 12 hours. In embodiments, the contacting of step (i) is for the duration of about 1.5 hours to about 12 hours. In embodiments, the contacting of step (i) is for the duration of about 2 hours to about 12 hours. In embodiments, the contacting of step (i) is for the duration of about 2.5 hours to about 12 hours. In embodiments, the contacting of step (i) is for the duration of about 3 hours to about 12 hours. In embodiments, the contacting of step (i) is for the duration of about 3.5 hours to about 12 hours. In embodiments, the contacting of step (i) is for the duration of about 4 hours to about 12 hours. In embodiments, the contacting of step (i) is for the duration of about 4.5 hours to about 12 hours. In embodiments, the contacting of step (i) is for the duration of about 5 hours to about 12 hours. In embodiments, the contacting of step (i) is for the duration of about 5.5 hours to about 12 hours. In embodiments, the contacting of step (i) is for the duration of about 6 hours to about 12 hours. In embodiments, the contacting of step (i) is for the duration of about 6.5 hours to about 12 hours. In embodiments, the contacting of step (i) is for the duration of about 7 hours to about 12 hours. In embodiments, the contacting of step (i) is for the duration of about 8 hours to about 12 hours. In embodiments, the contacting of step (i) is for the duration of about 8.5 hours to about 12 hours. In embodiments, the contacting of step (i) is for the duration of about 9 hours to about 12 hours. In embodiments, the contacting of step (i) is for the duration of about 9.5 hours to about 12 hours. In embodiments, the contacting of step (i) is for the duration of about 10 hours to about 12 hours. In embodiments, the contacting of step (i) is for the duration of about 10.5 hours to about 12 hours. In embodiments, the contacting of step (i) is for the duration of about 11 hours to about 12 hours.

In embodiments, the contacting of step (i) is for the duration of about 0.5 hours to about 11.5 hours. In embodiments, the contacting of step (i) is for the duration of about 0.5 hours to about 11 hours. In embodiments, the contacting of step (i) is for the duration of about 0.5 hours to about 10.5 hours. In embodiments, the contacting of step (i) is for the duration of about 0.5 hours to about 10 hours. In embodiments, the contacting of step (i) is for the duration of about 0.5 hours to about 9.5 hours. In embodiments, the contacting of step (i) is for the duration of about 0.5 hours to about 9 hours. In embodiments, the contacting of step (i) is for the duration of about 0.5 hours to about 8.5 hours. In embodiments, the contacting of step (i) is for the duration of about 0.5 hours to about 8 hours. In embodiments, the contacting of step (i) is for the duration of about 0.5 hours to about 7.5 hours. In embodiments, the contacting of step (i) is for the duration of about 0.5 hours to about 7 hours. In embodiments, the contacting of step (i) is for the duration of about 0.5 hours to about 6.5 hours. In embodiments, the contacting of step (i) is for the duration of about 0.5 hours to about 6 hours. In embodiments, the contacting of step (i) is for the duration of about 0.5 hours to about 5.5 hours. In embodiments, the contacting of step (i) is for the duration of about 0.5 hours to about 5 hours. In embodiments, the contacting of step (i) is for the duration of about 0.5 hours to about 4.5 hours. In embodiments, the contacting of step (i) is for the duration of about 0.5 hours to about 4 hours. In embodiments, the contacting of step (i) is for the duration of about 0.5 hours to about 3.5 hours. In embodiments, the contacting of step (i) is for the duration of about 0.5 hours to about 3 hours. In embodiments, the contacting of step (i) is for the duration of about 0.5 hours to about 2.5 hours. In embodiments, the contacting of step (i) is for the duration of about 0.5 hours to about 3 hours. In embodiments, the contacting of step (i) is for the duration of about 0.5 hours to about 2.5 hours. In embodiments, the contacting of step (i) is for the duration of about 0.5 hours to about 2 hours. In embodiments, the contacting of step (i) is for the duration of about 0.5 hours to about 1.5 hours. In embodiments, the contacting of step (i) is for the duration of about 12, about 11.5, about 11, about 10.5, about 10, about 9.5, about 9, about 8.5, about 8, about 7.5, about 7, about 6.5, about 6, about 5.5, about 5, about 4.5, about 4, about 3.5, about 3, about 2.5, about 2, about 1.5, or about 1 hour.

In embodiments, the PGC is formed within less than 4 days. In embodiments, the PGC is formed within about 0.5 days to about 4 days. In embodiments, the PGC is formed within about 1 day to about 4 days. In embodiments, the PGC is formed within about 1.5 days to about 4 days. In embodiments, the PGC is formed within about 2 days to about 4 days. In embodiments, the PGC is formed within about 2.5 days to about 4 days. In embodiments, the PGC is formed within about 3 days to about 4 days.

In embodiments, the PGC is formed within about 0.5 days to about 3.5 days. In embodiments, the PGC is formed within about 0.5 days to about 3 days. In embodiments, the PGC is formed within about 0.5 days to about 2.5 days. In embodiments, the PGC is formed within about 0.5 days to about 2 days. In embodiments, the PGC is formed within about 0.5 days to about 1.5 days. In embodiments, the PGC is formed within about 0.5 days, about 1 day, about 1.5 days, about 2 days, about 2.5 days, about 3 days, about 3.5 days, or about 4 days.

In embodiments, the contacting of step (i) includes expanding the pluripotent stem cell population. In embodiments, the contacting of step (ii) includes expanding the posterior epiblast cell population. The term “expanding” as provided herein is used according to its customary meaning in the biological arts and refers to allowing a cell (e.g., pluripotent stem cell) to divide and/or differentiate in a cell culture container in an appropriate cell culture medium.

In embodiments, the contacting of step (ii) further includes contacting the posterior epiblast cell population with a bone morphogenetic protein (BMP), stem cell factor (SCF), epidermal growth factor (EGF) or any combination thereof. In embodiments, the contacting of step (ii) further includes contacting the posterior epiblast cell population with a bone morphogenetic protein (BMP). In embodiments, the contacting of step (ii) further includes contacting the posterior epiblast cell population with a stem cell factor (SCF). In embodiments, the contacting of step (ii) further includes contacting the posterior epiblast cell population with a epidermal growth factor (EGF). In embodiments, the contacting of step (ii) further includes contacting the posterior epiblast cell population with a bone morphogenetic protein (BMP), stem cell factor (SCF) and an epidermal growth factor (EGF).

The term “BMP-4” or “BMP-4 protein” as provided herein includes any of the recombinant or naturally-occurring forms of the bone morphogenetic protein 4 (BMP-4) or variants or homologs thereof that maintain BMP-4 protein activity (e.g. within at least 50%, 80%, 90%, 95%, 96%, 97%, 98%, 99% or 100% activity compared to BMP-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BMP-4 polypeptide. In embodiments, BMP-4 is the protein as identified by the UniProt reference number P12644 or a variant or homolog having substantial identity thereto.

In embodiments, the posterior epiblast cell population is contacted with 15 ng/mL to 600 ng/mL BMP-4. In embodiments, the posterior epiblast cell population is contacted with 30 ng/mL to 600 ng/mL BMP-4. In embodiments, the posterior epiblast cell population is contacted with 45 ng/mL to 600 ng/mL BMP-4. In embodiments, the posterior epiblast cell population is contacted with 60 ng/mL to 600 ng/mL BMP-4. In embodiments, the posterior epiblast cell population is contacted with 75 ng/mL to 600 ng/mL BMP-4. In embodiments, the posterior epiblast cell population is contacted with 90 ng/mL to 600 ng/mL BMP-4. In embodiments, the posterior epiblast cell population is contacted with 105 ng/mL to 600 ng/mL BMP-4. In embodiments, the posterior epiblast cell population is contacted with 120 ng/mL to 600 ng/mL BMP-4. In embodiments, the posterior epiblast cell population is contacted with 135 ng/mL to 600 ng/mL BMP-4. In embodiments, the posterior epiblast cell population is contacted with 150 ng/mL to 600 ng/mL BMP-4. In embodiments, the posterior epiblast cell population is contacted with 165 ng/mL to 600 ng/mL BMP-4. In embodiments, the posterior epiblast cell population is contacted with 180 ng/mL to 600 ng/mL BMP-4. In embodiments, the posterior epiblast cell population is contacted with 195 ng/mL to 600 ng/mL BMP-4. In embodiments, the posterior epiblast cell population is contacted with 210 ng/mL to 600 ng/mL BMP-4. In embodiments, the posterior epiblast cell population is contacted with 225 ng/mL to 600 ng/mL BMP-4. In embodiments, the posterior epiblast cell population is contacted with 240 ng/mL to 600 ng/mL BMP-4. In embodiments, the posterior epiblast cell population is contacted with 255 ng/mL to 600 ng/mL BMP-4. In embodiments, the posterior epiblast cell population is contacted with 270 ng/mL to 600 ng/mL BMP-4. In embodiments, the posterior epiblast cell population is contacted with 285 ng/mL to 600 ng/mL BMP-4. In embodiments, the posterior epiblast cell population is contacted with 300 ng/mL to 600 ng/mL BMP-4. In embodiments, the posterior epiblast cell population is contacted with 315 ng/mL to 600 ng/mL BMP-4. In embodiments, the posterior epiblast cell population is contacted with 330 ng/mL to 600 ng/mL BMP-4. In embodiments, the posterior epiblast cell population is contacted with 345 ng/mL to 600 ng/mL BMP-4. In embodiments, the posterior epiblast cell population is contacted with 360 to 600 ng/mL BMP-4. In embodiments, the posterior epiblast cell population is contacted with 375 ng/mL to 600 ng/mL BMP-4. In embodiments, the posterior epiblast cell population is contacted with 390 ng/mL to 600 ng/mL BMP-4. In embodiments, the posterior epiblast cell population is contacted with 405 ng/mL to 600 ng/mL BMP-4. In embodiments, the posterior epiblast cell population is contacted with 420 to 600 ng/mL BMP-4. In embodiments, the posterior epiblast cell population is contacted with 435 ng/mL to 600 ng/mL BMP-4. In embodiments, the posterior epiblast cell population is contacted with 450 ng/mL to 600 ng/mL BMP-4. In embodiments, the posterior epiblast cell population is contacted with 465 ng/mL to 600 ng/mL BMP-4. In embodiments, the posterior epiblast cell population is contacted with 480 ng/mL to 600 ng/mL BMP-4. In embodiments, the posterior epiblast cell population is contacted with 495 ng/mL to 600 ng/mL BMP-4. In embodiments, the posterior epiblast cell population is contacted with 510 ng/mL to 600 ng/mL BMP-4. In embodiments, the posterior epiblast cell population is contacted with 525 ng/mL to 600 ng/mL BMP-4. In embodiments, the posterior epiblast cell population is contacted with 540 ng/mL to 600 ng/mL BMP-4. In embodiments, the posterior epiblast cell population is contacted with 555 ng/mL to 600 ng/mL BMP-4. In embodiments, the posterior epiblast cell population is contacted with 570 ng/mL to 600 ng/mL BMP-4. In embodiments, the posterior epiblast cell population is contacted with 585 ng/mL to 600 ng/mL BMP-4.

In embodiments, the posterior epiblast cell population is contacted with 15 ng/mL to 585 ng/mL BMP-4. In embodiments, the posterior epiblast cell population is contacted with 15 ng/mL to 570 ng/mL BMP-4. In embodiments, the posterior epiblast cell population is contacted with 15 ng/mL to 555 ng/mL BMP-4. In embodiments, the posterior epiblast cell population is contacted with 15 ng/mL to 540 ng/mL BMP-4. In embodiments, the posterior epiblast cell population is contacted with 15 ng/mL to 525 ng/mL BMP-4. In embodiments, the posterior epiblast cell population is contacted with 15 ng/mL to 510 ng/mL BMP-4. In embodiments, the posterior epiblast cell population is contacted with 15 ng/mL to 495 ng/mL BMP-4. In embodiments, the posterior epiblast cell population is contacted with 15 ng/mL to 480 ng/mL BMP-4. In embodiments, the posterior epiblast cell population is contacted with 15 ng/mL to 465 ng/mL BMP-4. In embodiments, the posterior epiblast cell population is contacted with 15 ng/mL to 450 ng/mL BMP-4. In embodiments, the posterior epiblast cell population is contacted with 15 ng/mL to 435 ng/mL BMP-4. In embodiments, the posterior epiblast cell population is contacted with 15 ng/mL to 420 ng/mL BMP-4. In embodiments, the posterior epiblast cell population is contacted with 15 ng/mL to 405 ng/mL BMP-4. In embodiments, the posterior epiblast cell population is contacted with 15 ng/mL to 390 ng/mL BMP-4. In embodiments, the posterior epiblast cell population is contacted with 15 ng/mL to 375 ng/mL BMP-4. In embodiments, the posterior epiblast cell population is contacted with 15 to 360 ng/mL BMP-4. In embodiments, the posterior epiblast cell population is contacted with 15 ng/mL to 345 ng/mL BMP-4. In embodiments, the posterior epiblast cell population is contacted with 15 ng/mL to 330 ng/mL BMP-4. In embodiments, the posterior epiblast cell population is contacted with 15 ng/mL to 315 ng/mL BMP-4. In embodiments, the posterior epiblast cell population is contacted with 15 ng/mL to 300 ng/mL BMP-4. In embodiments, the posterior epiblast cell population is contacted with 15 ng/mL to 285 ng/mL BMP-4. In embodiments, the posterior epiblast cell population is contacted with 15 ng/mL to 270 ng/mL BMP-4. In embodiments, the posterior epiblast cell population is contacted with 15 ng/mL to 255 ng/mL BMP-4. In embodiments, the posterior epiblast cell population is contacted with 15 ng/mL to 240 ng/mL BMP-4. In embodiments, the posterior epiblast cell population is contacted with 15 ng/mL to 225 ng/mL BMP-4. In embodiments, the posterior epiblast cell population is contacted with 15 ng/mL to 210 ng/mL BMP-4. In embodiments, the posterior epiblast cell population is contacted with 15 ng/mL to 195 ng/mL BMP-4. In embodiments, the posterior epiblast cell population is contacted with 15 ng/mL to 180 ng/mL BMP-4. In embodiments, the posterior epiblast cell population is contacted with 15 ng/mL to 165 ng/mL BMP-4. In embodiments, the posterior epiblast cell population is contacted with 15 ng/mL ng/mL to 150 ng/mL BMP-4. In embodiments, the posterior epiblast cell population is contacted with 15 ng/mL to 135 ng/mL BMP-4. In embodiments, the posterior epiblast cell population is contacted with 15 ng/mL to 120 ng/mL BMP-4. In embodiments, the posterior epiblast cell population is contacted with 15 ng/mL to 105 ng/mL BMP-4. In embodiments, the posterior epiblast cell population is contacted with 15 ng/mL to 90 ng/mL BMP-4. In embodiments, the posterior epiblast cell population is contacted with 15 ng/mL to 75 ng/mL BMP-4. In embodiments, the posterior epiblast cell population is contacted with 15 ng/mL to 60 ng/mL BMP-4. In embodiments, the posterior epiblast cell population is contacted with 15 ng/mL to 45 ng/mL BMP-4. In embodiments, the posterior epiblast cell population is contacted with 15 ng/mL to 30 ng/mL BMP-4.

In embodiments, the posterior epiblast cell population is contacted with about 15 ng/mL, 30 ng/mL, 45 ng/mL, 60 ng/mL, 75 ng/mL, 90 ng/mL, 105 ng/mL, 120 ng/mL, 135 ng/mL, 150 ng/mL, 165 ng/mL, 180 ng/mL, 195 ng/mL, 210 ng/mL, 225 ng/mL, 240 ng/mL, 255 ng/mL, 270 ng/mL, 285 ng/mL, 300 ng/mL, 315 ng/mL, 330 ng/mL, 345 ng/mL, 360 ng/mL, 375 ng/mL, 390 ng/mL, 405 ng/mL, 420 ng/mL, 435 ng/mL, 450 ng/mL, 465 ng/mL, 480 ng/mL, 495 ng/mL, 510 ng/mL, 525 ng/mL, 540 ng/mL, 555 ng/mL, 570 ng/mL, 585 ng/mL or 600 ng/mL BMP-4. In embodiments, the posterior epiblast cell population is contacted with 15 ng/mL, 30 ng/mL, 45 ng/mL, 60 ng/mL, 75 ng/mL, 90 ng/mL, 105 ng/mL, 120 ng/mL, 135 ng/mL, 150 ng/mL, 165 ng/mL, 180 ng/mL, 195 ng/mL, 210 ng/mL, 225 ng/mL, 240 ng/mL, 255 ng/mL, 270 ng/mL, 285 ng/mL, 300 ng/mL, 315 ng/mL, 330 ng/mL, 345 ng/mL, 360 ng/mL, 375 ng/mL, 390 ng/mL, 405 ng/mL, 420 ng/mL, 435 ng/mL, 450 ng/mL, 465 ng/mL, 480 ng/mL, 495 ng/mL, 510 ng/mL, 525 ng/mL, 540 ng/mL, 555 ng/mL, 570 ng/mL, 585 ng/mL or 600 ng/mL BMP-4. In embodiments, the posterior epiblast cell population is contacted with about 20 ng/mL BMP-4. In embodiments, the posterior epiblast cell population is contacted with 20 ng/mL BMP-4.

“XAV939” in a customary sense refers to the compound identified by Cas Registry No.: 284028-89-3 or the chemical name 3,5,7,8-Tetrahydro-2-[4-(trifluoromethyl)phenyl]-4H-thiopyrano[4,3-d]pyrimidin-4-one.

In embodiments, the pluripotent stem cell population is contacted with 0.1 µM to 5 µM XAV939. In embodiments, the pluripotent stem cell population is contacted with 0.5 µM to 5 µM XAV939. In embodiments, the pluripotent stem cell population is contacted with 1 µM to 5 µM XAV939. In embodiments, the pluripotent stem cell population is contacted with 1.5 µM to 5 µM XAV939. In embodiments, the pluripotent stem cell population is contacted with 2 µM to 5 µM XAV939. In embodiments, the pluripotent stem cell population is contacted with 2.5 µM to 5 µM XAV939. In embodiments, the pluripotent stem cell population is contacted with 3 µM to 5 µM XAV939. In embodiments, the pluripotent stem cell population is contacted with 3.5 µM to 5 µM XAV939. In embodiments, the pluripotent stem cell population is contacted with 4 µM to 5 µM XAV939.

In embodiments, the pluripotent stem cell population is contacted with 0.1 µM to 4.5 µM XAV939. In embodiments, the pluripotent stem cell population is contacted with 0.1 µM to 4 µM XAV939. In embodiments, the pluripotent stem cell population is contacted with 0.1 µM to 3.5 µM XAV939. In embodiments, the pluripotent stem cell population is contacted with 0.1 µM to 3 µM XAV939. In embodiments, the pluripotent stem cell population is contacted with 0.1 µM to 2.5 µM XAV939. In embodiments, the pluripotent stem cell population is contacted with 0.1 µM to 2 µM XAV939. In embodiments, the pluripotent stem cell population is contacted with 0.1 µM to 1.5 µM XAV939. In embodiments, the pluripotent stem cell population is contacted with 0.1 µM to 1 µM XAV939. In embodiments, the pluripotent stem cell population is contacted with 0.1 µM to 0.5 µM XAV939.

In embodiments, the pluripotent stem cell population is contacted with about 0.1 µM, 0.5 µM, 1 µM, 1.5 µM, 2 µM, 2.5 µM, 3 µM, 3.5 µM, 4 µM, 4.5 µM or 5 µM XAV939. In embodiments, the pluripotent stem cell population is contacted with 0.1 µM, 0.5 µM, 1 µM, 1.5 µM, 2 µM, 2.5 µM, 3 µM, 3.5 µM, 4 µM, 4.5 µM or 5 µM XAV939. In embodiments, the pluripotent stem cell population is contacted with about 1 µM XAV939. In embodiments, the pluripotent stem cell population is contacted with 1 µM XAV939.

The term “SCF” or “SCF protein” as provided herein includes any of the recombinant or naturally-occurring forms of the stem cell factor (SCF), also known as Kit ligand, Mast cell growth factor, c-Kit ligand or variants or homologs thereof that maintain BMP-4 protein activity (e.g. within at least 50%, 80%, 90%, 95%, 96%, 97%, 98%, 99% or 100% activity compared to BMP-4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BMP-4 polypeptide. In embodiments, BMP-4 is the protein as identified by the UniProt reference number P21583 or a variant or homolog having substantial identity thereto.

In embodiments, the posterior epiblast cell population is contacted with 50 ng/ml to 300 ng/ml SCF. In embodiments, the posterior epiblast cell population is contacted with 75 ng/ml to 300 ng/ml SCF. In embodiments, the posterior epiblast cell population is contacted with 100 ng/ml to 300 ng/ml SCF. In embodiments, the posterior epiblast cell population is contacted with 125 ng/ml to 300 ng/ml SCF. In embodiments, the posterior epiblast cell population is contacted with 150 ng/ml to 300 ng/ml SCF. In embodiments, the posterior epiblast cell population is contacted with 175 ng/ml to 300 ng/ml SCF. In embodiments, the posterior epiblast cell population is contacted with 200 ng/ml to 300 ng/ml SCF. In embodiments, the posterior epiblast cell population is contacted with 225 ng/ml to 300 ng/ml SCF. In embodiments, the posterior epiblast cell population is contacted with 250 ng/ml to 300 ng/ml SCF. In embodiments, the posterior epiblast cell population is contacted with 275 ng/ml to 300 ng/ml SCF.

In embodiments, the posterior epiblast cell population is contacted with 50 ng/ml to 275 ng/ml SCF. In embodiments, the posterior epiblast cell population is contacted with 50 ng/ml to 250 ng/ml SCF. In embodiments, the posterior epiblast cell population is contacted with 50 ng/ml to 225 ng/ml SCF. In embodiments, the posterior epiblast cell population is contacted with 50 ng/ml to 200 ng/ml SCF. In embodiments, the posterior epiblast cell population is contacted with 50 ng/ml to 175 ng/ml SCF. In embodiments, the posterior epiblast cell population is contacted with 50 ng/ml to 150 ng/ml SCF. In embodiments, the posterior epiblast cell population is contacted with 50 ng/ml to 125 ng/ml SCF. In embodiments, the posterior epiblast cell population is contacted with 50 ng/ml to 100 ng/ml SCF. In embodiments, the posterior epiblast cell population is contacted with 50 ng/ml to 75 ng/ml SCF. In embodiments, the posterior epiblast cell population is contacted with 50 ng/ml to 50 ng/ml SCF. In embodiments, the posterior epiblast cell population is contacted with 50 ng/ml to 25 ng/ml SCF.

In embodiments, the posterior epiblast cell population is contacted with about 50 ng/ml, 75 ng/ml, 100 ng/ml, 125 ng/ml, 150 ng/ml, 175 ng/ml, 200 ng/ml, 225 ng/ml, 250 ng/ml, 275 ng/ml or 300 ng/ml SCF. In embodiments, the posterior epiblast cell population is contacted with 50 ng/ml, 75 ng/ml, 100 ng/ml, 125 ng/ml, 150 ng/ml, 175 ng/ml, 200 ng/ml, 225 ng/ml, 250 ng/ml, 275 ng/ml or 300 ng/ml SCF. In embodiments, the posterior epiblast cell population is contacted with about 100 ng/ml SCF. In embodiments, the posterior epiblast cell population is contacted with 100 ng/ml SCF.

The term “EGF” or “EGF protein” as provided herein includes any of the recombinant or naturally-occurring forms of the epidermal growth factor (EGF), also known as Urogastrone or variants or homologs thereof that maintain EGF protein activity (e.g. within at least 50%, 80%, 90%, 95%, 96%, 97%, 98%, 99% or 100% activity compared to EGF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGF polypeptide. In embodiments, EGF is the protein as identified by the UniProt reference number P01133 or a variant or homolog having substantial identity thereto.

In embodiments, the posterior epiblast cell population is contacted with 50 ng/ml to 250 ng/ml EGF. In embodiments, the posterior epiblast cell population is contacted with 75 ng/ml to 250 ng/ml EGF. In embodiments, the posterior epiblast cell population is contacted with 100 ng/ml to 250 ng/ml EGF. In embodiments, the posterior epiblast cell population is contacted with 125 ng/ml to 250 ng/ml EGF. In embodiments, the posterior epiblast cell population is contacted with 150 ng/ml to 250 ng/ml EGF. In embodiments, the posterior epiblast cell population is contacted with 175 ng/ml to 250 ng/ml EGF. In embodiments, the posterior epiblast cell population is contacted with 200 ng/ml to 250 ng/ml EGF. In embodiments, the posterior epiblast cell population is contacted with 225 ng/ml to 250 ng/ml EGF.

In embodiments, the posterior epiblast cell population is contacted with 50 ng/ml to 225 ng/ml EGF. In embodiments, the posterior epiblast cell population is contacted with 50 ng/ml to 200 ng/ml EGF. In embodiments, the posterior epiblast cell population is contacted with 50 ng/ml to 175 ng/ml EGF. In embodiments, the posterior epiblast cell population is contacted with 50 ng/ml to 150 ng/ml EGF. In embodiments, the posterior epiblast cell population is contacted with 50 ng/ml to 125 ng/ml EGF. In embodiments, the posterior epiblast cell population is contacted with 50 ng/ml to 100 ng/ml EGF. In embodiments, the posterior epiblast cell population is contacted with 50 ng/ml to 75 ng/ml EGF.

In embodiments, the posterior epiblast cell population is contacted with about 50 ng/ml, 75 ng/ml, 100 ng/ml, 125 ng/ml, 150 ng/ml, 175 ng/ml, 200 ng/ml, 225 ng/ml or 250 ng/ml EGF. In embodiments, the posterior epiblast cell population is contacted with 50 ng/ml, 75 ng/ml, 100 ng/ml, 125 ng/ml, 150 ng/ml, 175 ng/ml, 200 ng/ml, 225 ng/ml or 250 ng/ml EGF. In embodiments, the posterior epiblast cell population is contacted with about 50 ng/ml EGF. In embodiments, the posterior epiblast cell population is contacted with 50 ng/ml EGF.

In embodiments, the contacting the epiblast cell population with the WNT inhibitor, BMP, SCF, EGF or any combination thereof occurs sequentially, or simultaneously. In embodiments, the contacting the epiblast cell population with the WNT inhibitor, BMP, SCF, EGF or any combination thereof occurs sequentially. In embodiments, the contacting the epiblast cell population with the WNT inhibitor, BMP, SCF, EGF or any combination thereof occurs simultaneously.

In embodiments, the pluripotent stem cell population forms a monolayer in a cell culture vessel. In embodiments, the pluripotent stem cell population forms a monolayer in a cell culture container. In embodiments, the posterior epiblast cell population expresses one or more pluripotency marker genes and one or more primitive-streak marker genes.

In embodiments, the posterior epiblast cell expresses one or more of Nodal, Eomes, GSC, Lin28a, Fgf8, Sox2, Pouf5fl/Oct4, Nanog, Fst, Brachyury/T, Mixl1, Sp5Id1, Lefty 2, Foxh1 or Fgf19. In embodiments, the posterior epiblast cell expresses Nodal. In embodiments, the posterior epiblast cell expresses Eomes. In embodiments, the posterior epiblast cell expresses GSC. In embodiments, the posterior epiblast cell expresses Lin28a. In embodiments, the posterior epiblast cell expresses Fgf8. In embodiments, the posterior epiblast cell expresses Sox2. In embodiments, the posterior epiblast cell expresses Pouf5fl/Oct4. In embodiments, the posterior epiblast cell expresses Nanog. In embodiments, the posterior epiblast cell expresses Fst. In embodiments, the posterior epiblast cell expresses Brachyury/T. In embodiments, the posterior epiblast cell expresses Mixl1. In embodiments, the posterior epiblast cell expresses Sp5Id1. In embodiments, the posterior epiblast cell expresses Lefty 2. In embodiments, the posterior epiblast cell expresses Foxh1. In embodiments, the posterior epiblast cell expresses Fgfl9.

In embodiments, the contacting of step (i) and step (ii) occurs in serum-free medium. In embodiments, the contacting of step (i) or step (ii) occurs in serum-free medium. In embodiments, the contacting of step (i) occurs in serum-free medium. In embodiments, the contacting of step (ii) occurs in serum-free medium.

In embodiments, the pluripotent stem cell population is a human pluripotent stem cell population or a human embryonic stem cell population. In embodiments, the pluripotent stem cell population is a human pluripotent stem cell population. In embodiments, the pluripotent stem cell population is a human embryonic stem cell population.

### Cell Compositions

In an aspect, a primordial germ cell formed by a method provided herein including embodiments thereof is provided.

In an aspect is provided an in vitro cell culture composition, including a WNT-activated posterior epiblast cell population in a cell culture medium comprising a WNT inhibitor.

In embodiments, the cell culture medium further includes BMP, SCF, EGF or any combination thereof. In embodiments, the cell culture medium further includes BMP. In embodiments, the cell culture medium further includes SCF. In embodiments, the cell culture medium further includes EGF.

In an aspect, an in vitro cell culture, including a WNT-activated posterior epiblast cell population in a cell culture medium including a WNT inhibitor is provided. A “WNT-activated” posterior epiblast cell as referred to herein is a posterior epiblast cell, which has been contacted with a WNT agonist. In embodiments, the WNT-activated posterior epiblast cell includes an activated WNT signaling pathway component.

In an aspect, an in vitro cell culture, including a CXCR4+/PDGFRα-/GARP¬ PGC in a cell culture medium including a Wnt inhibitor is provided. Any of the WNT inhbitors described herein is contemplated for the cell compositions described in this section.

In embodiments, the PGC expresses one or more of the proteins selected from OCT4, NANOG, TFCP2L1, BLIMP1, NANOS3 and TFAP2C (AP2y). In embodiments, the PGC does not express detectable amounts of FOXA2, HHEX or extraembryonic fate (CDX2). In embodiments, the cell culture medium further includes BMP, SCF, EGF or any combination thereof.

In embodiments, the cell culture is in a cell culture container. In embodiments, the cell culture forms a monolayer. In embodiments, the cell culture medium is a serum-free medium.

In an aspect, a primordial germ cell in a cell culture container including a cell culture medium including a WNT inhibitor is provided, wherein the PGC is a CXCR4+/PDGFRα-/GARP¬ PGC. In embodiments, the PGC expresses one or more of the proteins selected from OCT4, NANOG, TFCP2L1, BLIMP1, NANOS3 and TFAP2C (AP2y). In embodiments, the PGC does not express detectable amounts of FOXA2, HHEX or extraembryonic fate (CDX2). In embodiments, the cell culture medium further comprises BMP, SCF, EGF or any combination thereof. In embodiments, the PGC forms part of a cell monolayer. In embodiments, the cell culture medium is a serum-free medium.

### METHODS OF USE

The PGCs provided herein including embodiments thereof may, inter alia, be in vitro differentiated to mature eggs and sperm and used for in vitro fertilization followed by transplantation of embryos in utero. The PGCs provided herein including embodiments thereof may, inter alia, be transplanted in vivo into ovaries and/or testes to restore fertility by repopulating the endogenous population of germ cells in the gonads. Further, the PGCs provided herein including embodiments thereof may be transplanted in vivo for the purpose of ameliorating immune functionality and beneficially impacting aging-associated morbidity.

In an aspect is provided a method of treating infertility in a subject in need thereof, the method including administering a therapeutically effective amount of a PGC provided herein including embodiments thereof to the subject, thereby treating infertility in the subject.

### EXAMPLES

### Example 1

Generating primordial germ cells (PGCs) from human pluripotent stem cells (hPSCs) advances studies of human reproduction and development of infertility treatments, but currently entails complex 3D aggregates. Here we develop a simplified, monolayer method to differentiate hPSCs into PGCs within 3.5 days, with higher efficiencies and improved consistency across multiple hPSC lines. Using our simplified platform and single-cell RNA-sequencing, we find that transient WNT activation for 12 hours differentiates hPSCs into posterior epiblast, a unique state wherein pluripotency and primitive-streak genes are coexpressed. Subsequently, sharp WNT inhibition together with BMP activation specifies PGCs; by contrast, continued WNT instead induces primitive streak. Thus, primitive streak and PGCs are related-yet distinct-lineages segregated by temporally-dynamic signaling. Pluripotency factors are continuously expressed during the transition from pluripotency to posterior epiblast to PGCs, thus bridging pluripotent and germline states. Finally, hPSC-derived PGCs can be easily purified by virtue of their CXCR4⁺PDGFRA⁻GARP⁻ surface-marker profile.

### Introduction

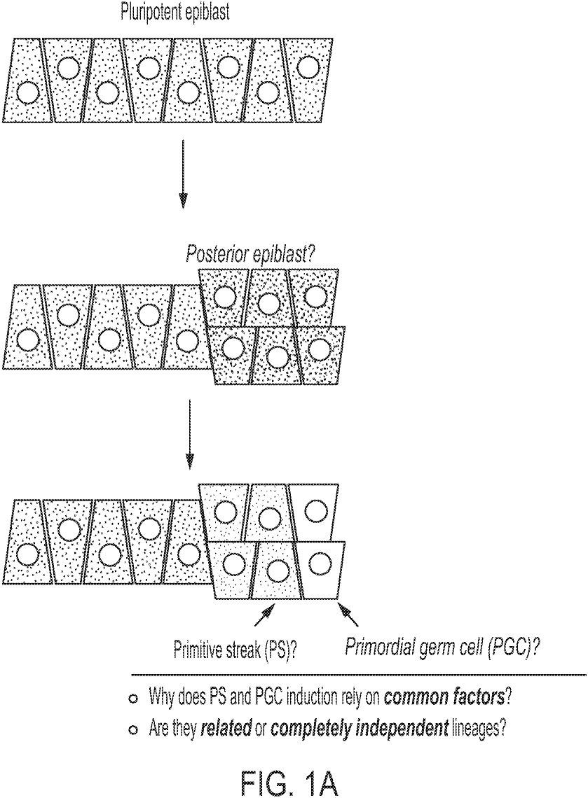

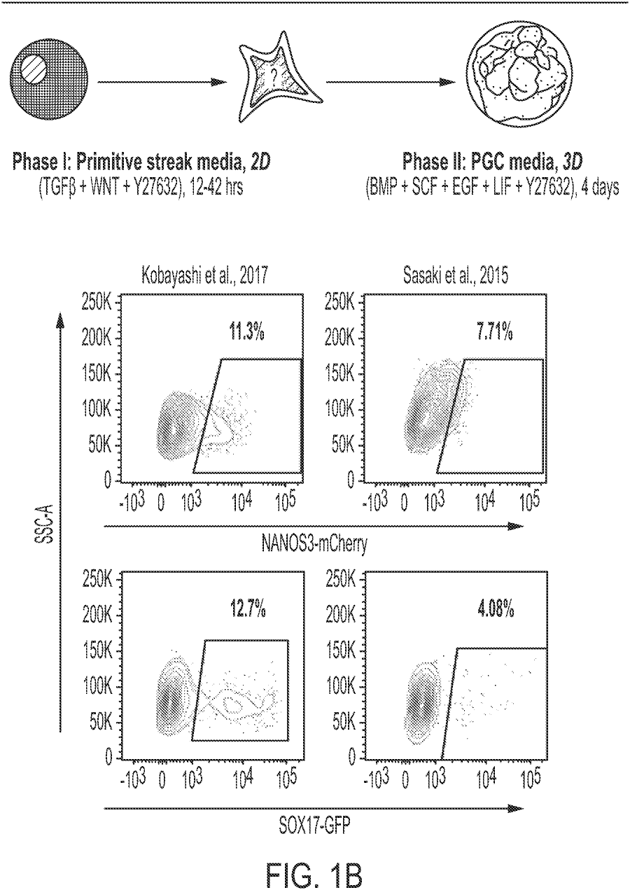

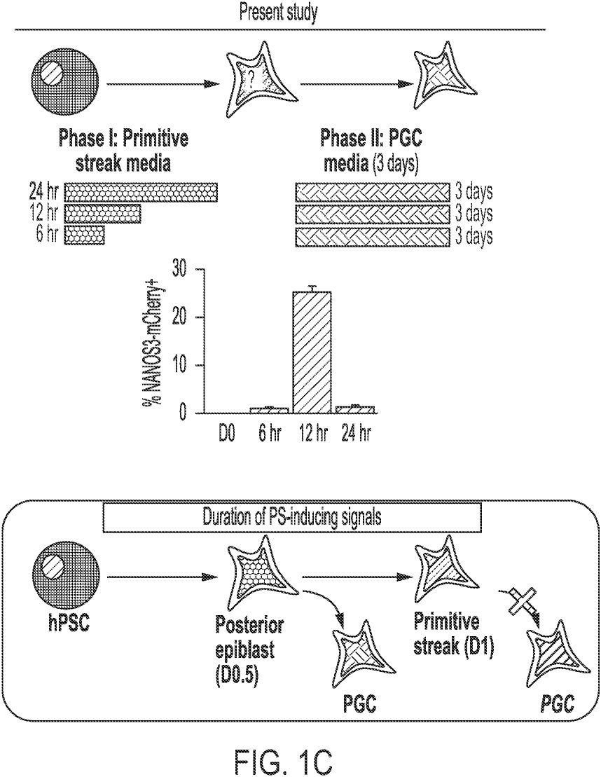

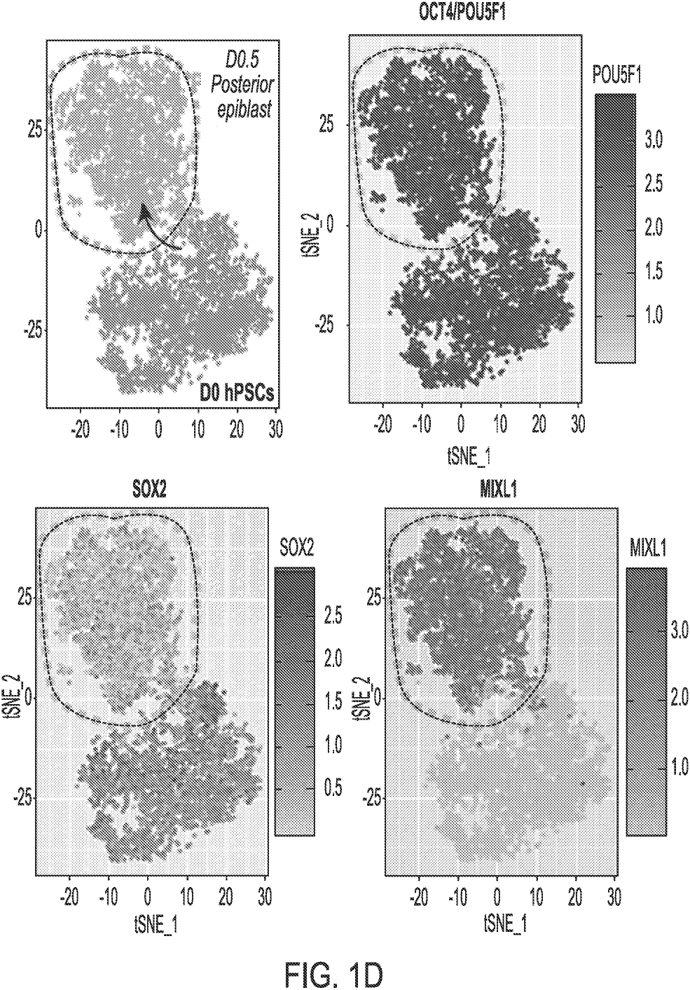

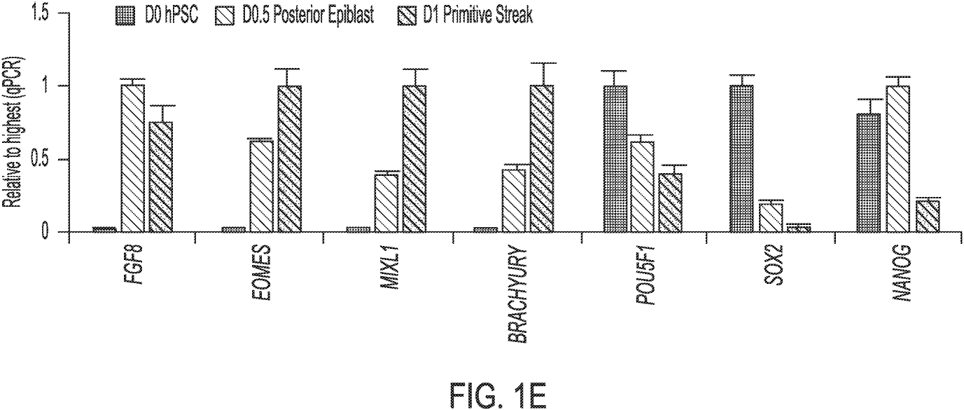

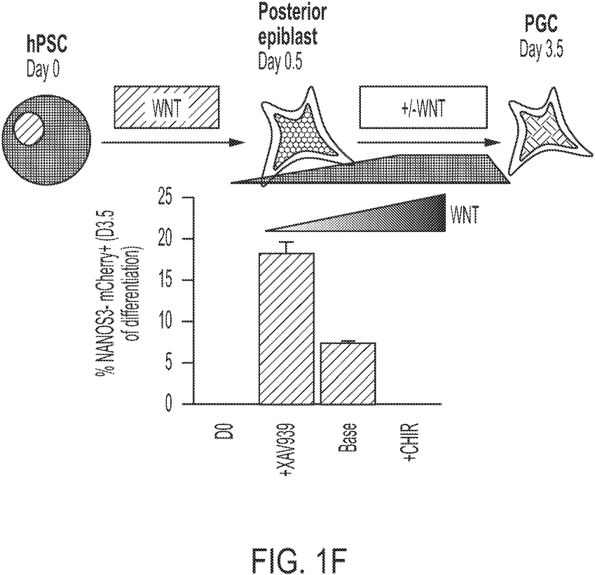

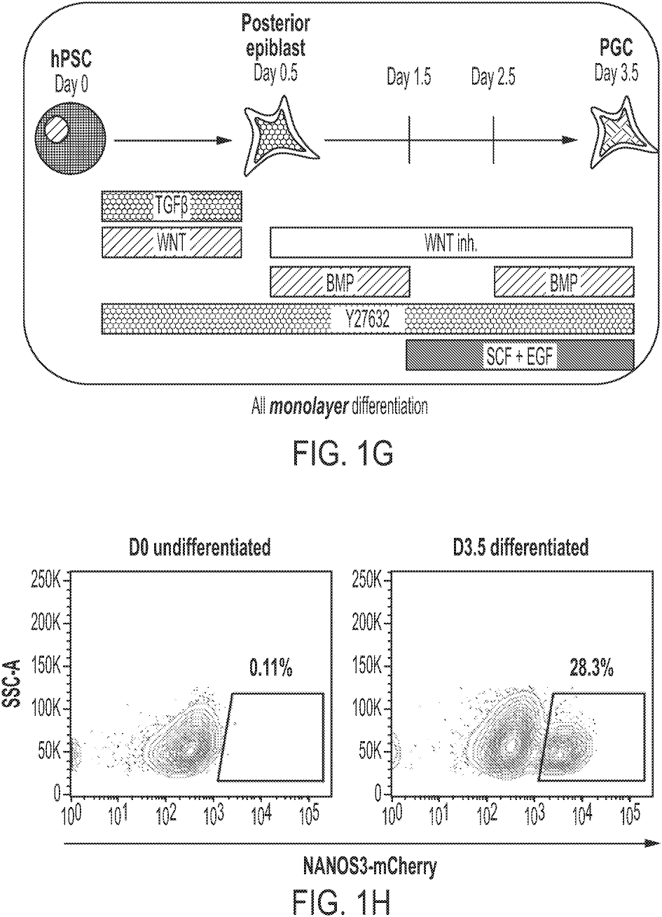

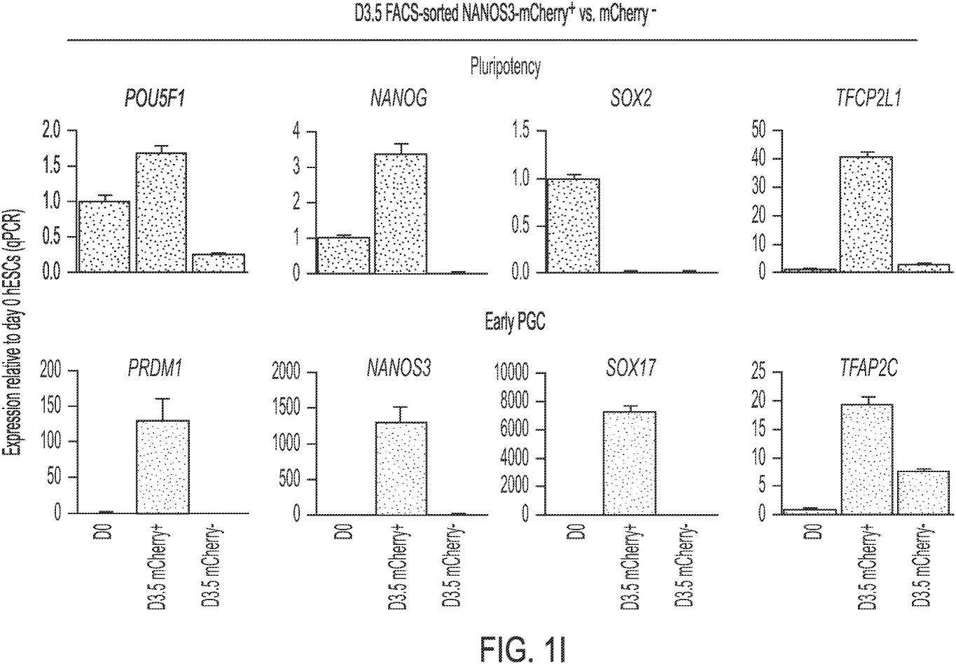

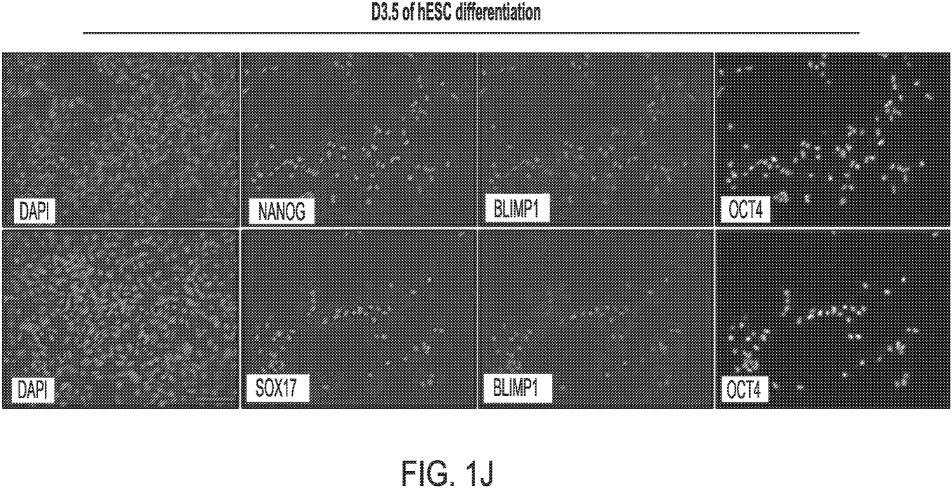

Within the mammalian embryo, PGCs are the harbinger to eggs and sperm; consequently, they are key to the act of reproduction itself and the vertical transmission of genetic and epigenetic information to the next generation. The origins of mouse PGCs have been thoroughly explored (Chiquoine, 1954; Ginsburg et al., 1990; Lawson and Hage, 1994), which has enabled the successful, stepwise differentiation of mouse pluripotent cells into PGCs in vitro (Hayashi et al., 2011; Ohinata et al., 2009). Yet, embryonic development of humans and mice has diverged across ~170 million years (Kobayashi and Surani, 2018), and fundamental differences in post-implantation development exist between these two species. In mice, soon after implantation the pluripotent epiblast forms a cup-shaped structure (the egg cylinder), and PGCs originate from the posterior region of the epiblast, in the vicinity of the primitive streak (the precursor to endoderm and mesoderm, FIG. 1A). Conversely, in human embryos, the epiblast is a flat bilaminar epithelium (the embryonic disc), and PGCs first arise within weeks 2-3 of human embryogenesis, presumably also in the posterior part of the embryo (De Felici, 2016; O′Rahilly and Müller, 1987). Ethical and technical hurdles in attaining early post-implantation human embryos limit our ability to decipher the precise origins of human PGCs and the signals that specify their formation, which have remained hitherto uncertain. Studies of pig embryos also suggest that PGCs form in the posterior epiblast, in the vicinity of the primitive streak (Kobayashi et al., 2017) (FIG. 1A). In cynomolgus monkey embryos, PGCs are instead thought to first form in the dorsal amnion (which also derives from the epiblast), and subsequently migrate into the yolk sac (Sasaki et al., 2016).

The exact relationship between PGCs and the primitive streak remains mysterious (FIG. 1A). Traditionally, PGCs and primitive streak have been regarded as two completely distinct lineages; in fact a hallmark of PGC development is “repression of somatic genes”, including primitive streak/mesodermal markers (Surani et al., 2007). Nonetheless, PGCs and primitive streak are specified by several common factors. First, the WNT and BMP signaling pathways—which are critical for primitive streak formation (Loh et al., 2014; Loh et al., 2016)—are also critical for PGC specification, and this is conserved across mammalian species studied thus far (Hayashi et al., 2011; Kobayashi et al., 2017; Ohinata et al., 2009; Sasaki et al., 2016). However, the precise temporal requirements for WNT and BMP signals in PGC specification remain to be elucidated. Second, Brachyury and Eomes (transcription factors expressed in, and important for, the primitive streak) are respectively required for mouse (Aramaki et al., 2013) and human (Chen et al., 2017; Kojima et al., 2017) PGC specification. One interpretation of these results is that PGCs may arise from a primitive streak/mesoderm-like intermediate (Sasaki et al., 2015). Alternatively, PGCs and primitive streak may be two completely independent lineages that coincidentally share several common developmental regulators. Taken together, multiple outstanding questions continue to surround where, when and how human PGCs are specified.

Given the uncertainties surrounding human PGC origins, preceding methods to generate human PGC-like cells from PSCs generated heterogeneous cell populations containing only a subset of PGC-like cells and relied on complex 3D cultures with high concentrations of growth factors (Irie et al., 2014; Kobayashi et al., 2017; Sasaki et al., 2015). Published protocols to differentiate hPSCs into PGCs show wide line-to-line variability (Yokobayashi et al., 2017), with a systematic comparison of multiple hPSC lines showing that, on average, less than 10% pure PGCs are generated (Chen et al., 2017). These complicates the generation of PGCs from hPSCs and our ability to dissect the precise mechanism of human PGC specification, including developmental intermediates and the precise timings and combinations of key extracellular signals.

Here we develop a simplified 2D in vitro platform to generate human PGCs within 3.5 days of hPSC differentiation, with the dual goals of better understanding the key lineage intermediates and extracellular signals in PGC specification and to simplify the production of human PGCs for future drug discovery, disease modeling and perhaps infertility treatments. First, we find that primed PSCs are competent to generate PGCs, and they first briefly differentiate into posterior epiblast (in 12 hours) before splitting off from the primitive streak lineage trajectory and then committing to form PGCs. Second, these differentiation steps entail the temporally dynamic activation and repression of the WNT and BMP pathways and this method can be robustly extrapolated to a wide range of hESC and hiPSC lines. Third, we also describe unique cell-surface markers for human PGC-like cells (namely, CXCR4⁺PDGFRA⁻GARP⁻) that enable their ready purification via flow cytometry. Finally, we provide a detailed characterization of cells en route to PGC specification by single cell RNA-seq analysis and find that pluripotency transcription factors OCT4 and NANOG are continuously expressed during this process, thus bridging pluripotency with germline states.

### Results

Brief exposure of pluripotent cells to primitive streak-inducing signals generates posterior epiblast cells with PGC potential. As a starting point, we first evaluated two published methods to differentiate hPSCs into PGCs (Kobayashi et al., 2017; Sasaki et al., 2015) and tested them on two hPSC reporter lines wherein fluorescent reporters were knocked into endogenous PGC marker genes: NANOS3-mCherry (Irie et al., 2014) and SOX17-GFP (Wang et al., 2011) (Fig. 1b). NANOS3 and SOX17 are both markers of human PGCs (Irie et al., 2014; Sasaki et al., 2015), although SOX17 is less specific, as it is also expressed in endoderm (Loh et al., 2014; Wang et al., 2011).

Both published methods to differentiate hPSCs into PGCs are divided into two phases (FIG. 1B). First, hPSCs are exposed to posteriorizing, primitive streak (PS)-inducing signals for 42 hours (Sasaki et al., 2015) or 12 hours (Kobayashi et al., 2017). Second, cells are then aggregated in 3D and are treated with high concentrations of BMP, EGF, SCF and LIF together with a non-specific ROCK inhibitor (Y27632) for 4 days (Kobayashi et al., 2017; Sasaki et al., 2015). In our hands, prolonged differentiation of hPSCs into PS for 42 hours at the first phase of differentiation (Sasaki et al., 2015) failed to reproducibly generate PGCs by the second stage (FIG. 1B). However, brief 12-hour exposure of hPSCs to PS-inducing signals was effective in generating PGCs (Kobayashi et al., 2017), albeit at lower frequencies compared to the original report (FIG. 1B). Based on these initial observations, we expanded upon the latter method, with emphasis on increasing differentiation efficiencies while obviating the need for complex 3D aggregates (Kobayashi et al., 2017). We used NANOS3-mCherry hPSCs to screen different culture conditions.

First, we determined the optimal exposure of hPSCs to PS-inducing signals at the first stage of differentiation. We found that treating hPSCs with TGFβ agonist (ACTIVIN), WNT agonist (CHIR99021) and Y27632 for 12 hours (Kobayashi et al., 2017) at the first stage was optimal in order to subsequently generate NANOS3-mCherry⁺ PGCs at the second stage of differentiation as monolayers (FIG. 1C). We henceforth refer to these intermediate cells generated upon 12-hour exposure to PS-inducing signals as “posterior epiblast”. This is consistent with how, in mouse embryos, the post-implantation pluripotent epiblast is formed by embryonic day 5.5 (E5.5), but then PS markers (e.g., Brachyury) are transiently expressed in the posterior region of the epiblast (~E6-E6.25) immediately prior to overt formation of the morphologically-conspicuous PS (~E6.5) (Rivera-Perez and Magnuson, 2005); similar results have been reported in pig embryos (Kobayashi et al., 2017). By contrast, treating hPSCs with these same signals (ACTIVIN, CHIR99021 and Y27632) for 24 hours—which we have shown generates fully-fledged PS cells (Kobayashi et al., 2017; Loh et al., 2014; Loh et al., 2016)—prevented subsequent differentiation into PGCs (FIG. 1C). In summary, pluripotent cells briefly exposed to PS-inducing signals become posterior epiblast cells, which then bifurcate in fate: cells can divert to become PGCs, but prolonged exposure to PS-inducing signals generates fully-fledged PS cells that become primed for endoderm and mesoderm differentiation (FIG. 1C). Taken together, this may reconcile the controversial relationship of the primitive streak and PGCs.

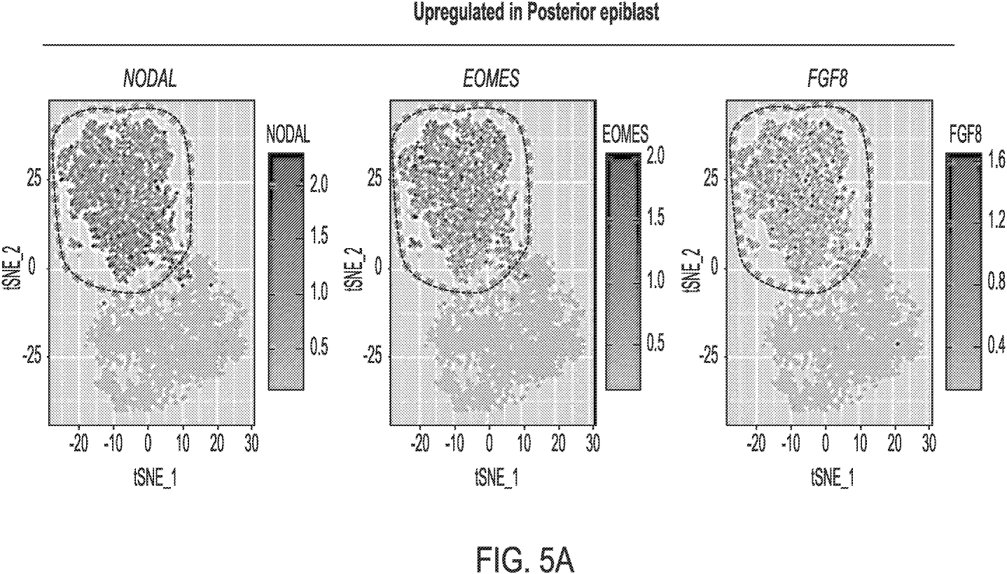

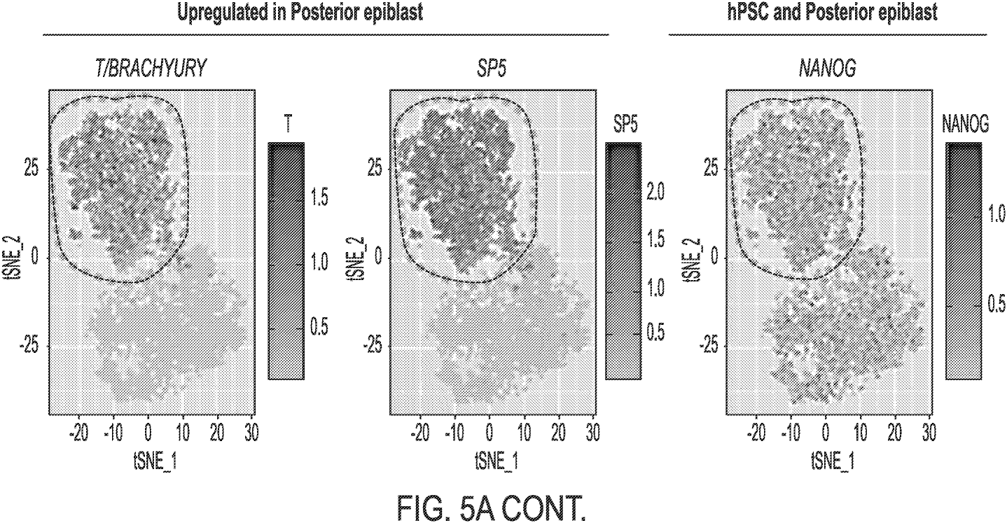

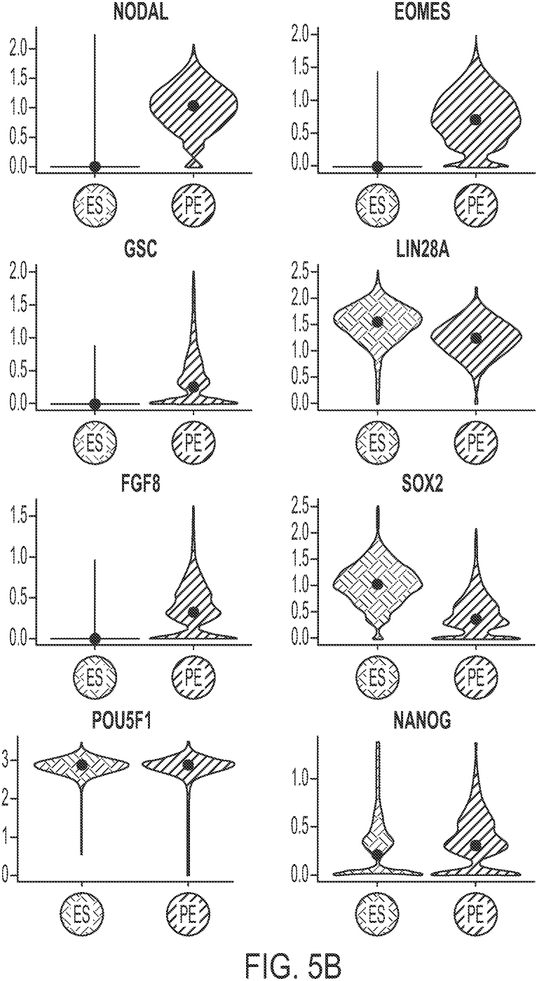

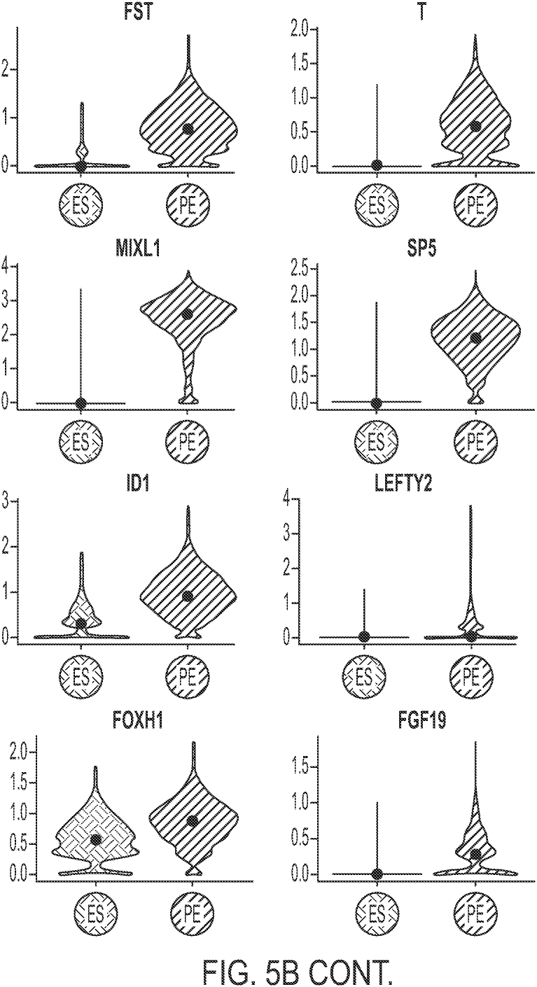

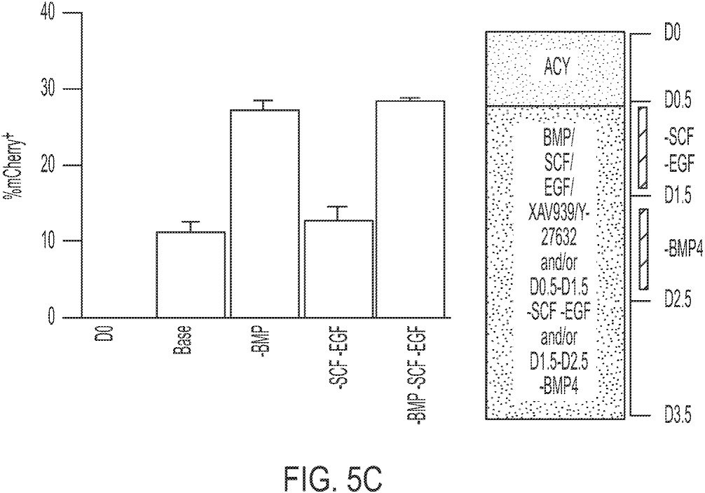

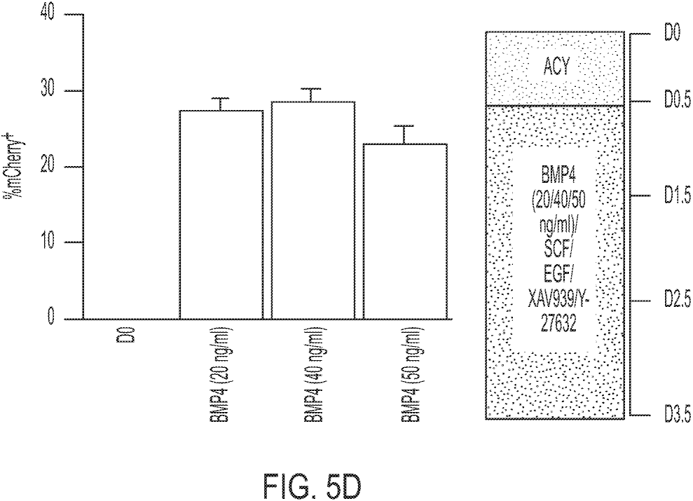

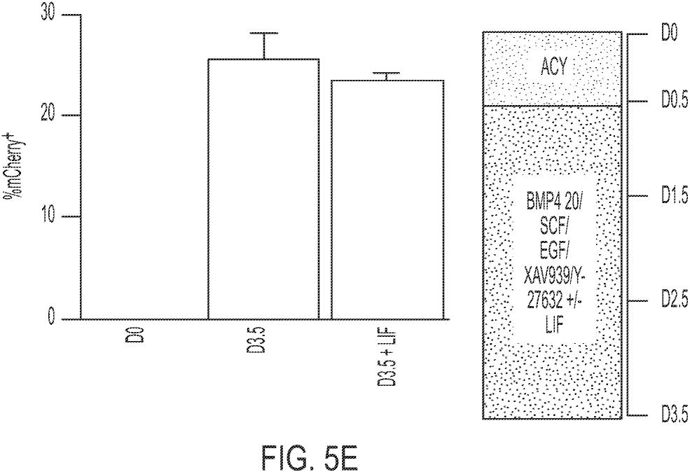

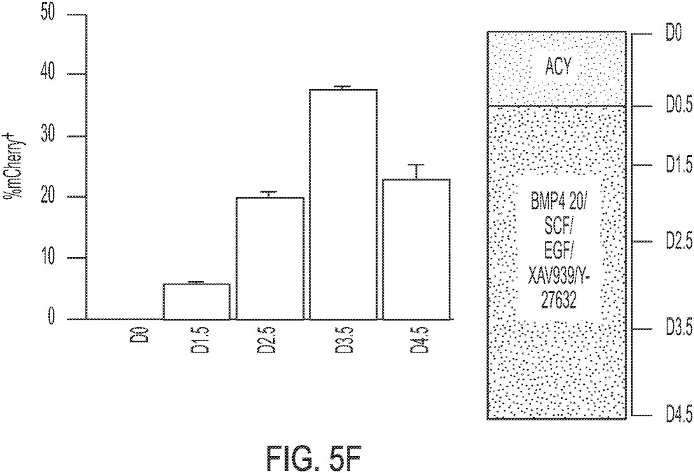

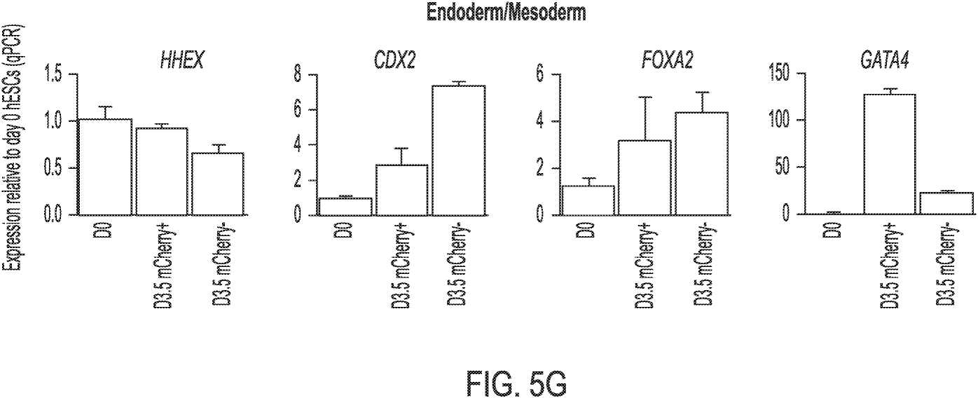

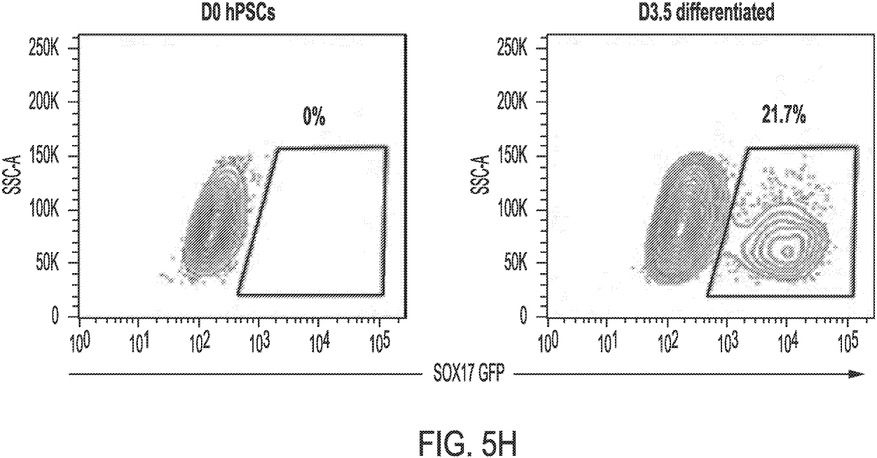

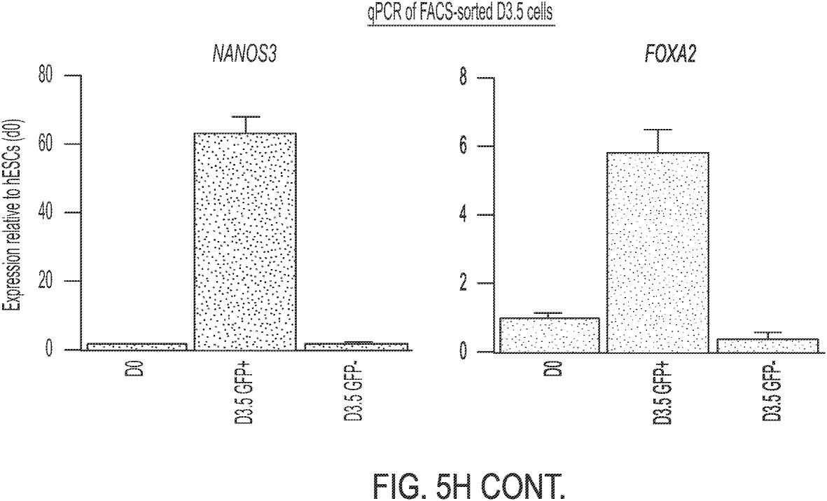

Given the unique competence of these incipient posterior epiblast cells (but not primitive streak) to give rise to PGCs, we sought to molecularly describe in more detail this transient intermediate state. Single-cell RNA-sequencing (scRNAseq) revealed that day 0.5 (D0.5) hPSC-derived posterior epiblast cells continued to highly express pluripotency transcription factors OCT4 and NANOG, although SOX2 began to decrease (FIG. 1D, FIGS. 5A, 5B). Posterior epiblast also began to concomitantly express posterior epiblast/future primitive streak markers such as MIXL1,BRACHYURY, FGF8 and NODAL (FIG. 1D, FIGS. 5A, 5B), although generally at lower levels as compared to D1 PS cells (FIG. 1E). Consistent with the use of TGFβ and WNT agonists to induce hPSC-derived posterior epiblast, such cells demonstrated an active transcriptional response to both signaling pathways, including TGFβ target genes (FOLLISTATIN, ID1 and LEFTY2) and WNT target genes (SP5) (FIG. 1D, FIGS. 5A, 5B). In summary, this discloses a unique transcriptional signature for posterior epiblast cells, where OCT4 and NANOG are co-expressed together with primitive streak markers.

Temporally dynamic WNT signaling drives progression of human pluripotent cells into posterior epiblast and then PGC-like cells. After generating D0.5 posterior epiblast cells, next we sought to optimize their subsequent differentiation into PGCs (the second phase of differentiation). Initial WNT activation for 12 hours differentiated hPSCs into posterior epiblast (FIG. 1C), but we hypothesized that later, WNT signaling has a temporally dynamic role in germline specification. We thus tested whether further activation or inhibition of WNT signaling (after the first 12 hours of activation) had an effect. Remarkably, sharp inhibition of WNT signaling (leaving all the other culture conditions unaltered) was sufficient to drastically enhance the yield of PGC formation by ~3-fold (FIG. 1F). Conversely, prolonged activation of WNT blocked PGC formation (FIG. 1F). These findings demonstrate that WNT initially promotes, and then sharply inhibits PGC specification in a temporally-dynamic fashion. Consequently, inhibiting WNT after the first 12 hours diverts posterior epiblast cells away from the PS, instead differentiating them into PGCs. The temporally-dynamic role of WNT explains why protocols that activate WNT for 42 sustained hours during PGC differentiation (Sasaki et al., 2015) generate low numbers of PGCs (FIG. 1B). Our results that late-stage WNT repression promotes PGC specification is consistent with how, after the posterior epiblast has formed, WNT is dispensable for PGC formation in pig embryonic explants (Kobayashi et al., 2017).

After demonstrating a positive effect of WNT inhibitors, we tested whether continuous BMP, SCF, LIF and EGF activation (Irie et al., 2014; Kobayashi et al., 2017; Sasaki et al., 2015) was required for the entire second phase of PGC differentiation. First, omitting BMP4 from the culture media for 24 hrs at day 2 of differentiation was sufficient to promote the differentiation of PGCs from about 10% to 30% while the absence of SCF and EGF in the same time window was superfluous (FIG. 5C). Second, past 3D differentiation methods used extremely high BMP4 concentrations (200-500 ng/mL) (Irie et al., 2014; Kobayashi et al., 2017), but in our monolayer conditions, we observed that significantly (25-fold) lower BMP4 concentrations were needed (FIG. 5D). This is consistent with the notion that BMP signaling does not effectively diffuse through large hPSC clusters (Warmflash et al., 2014; Zhang et al., 2019). 3D aggregates may therefore impair BMP signaling, thus emphasizing the benefits of a monolayer differentiation system. Third, LIF, which is commonly used to enhance PGC survival (Irie et al., 2014; Kobayashi et al., 2017; Sasaki et al., 2015), was dispensable in our platform (FIG. 5E). Fourth, we observed a peak of PGC formation by day 3.5 of differentiation (FIG. 1F).

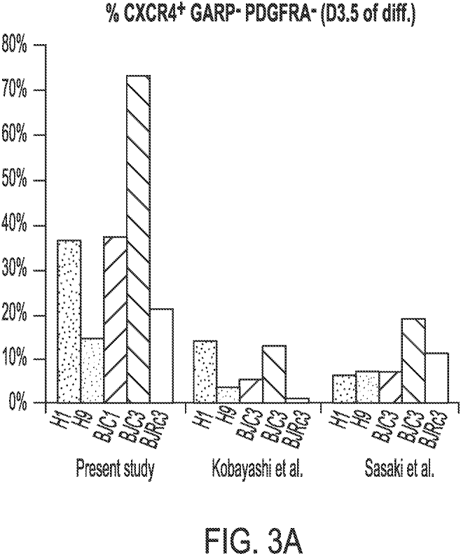

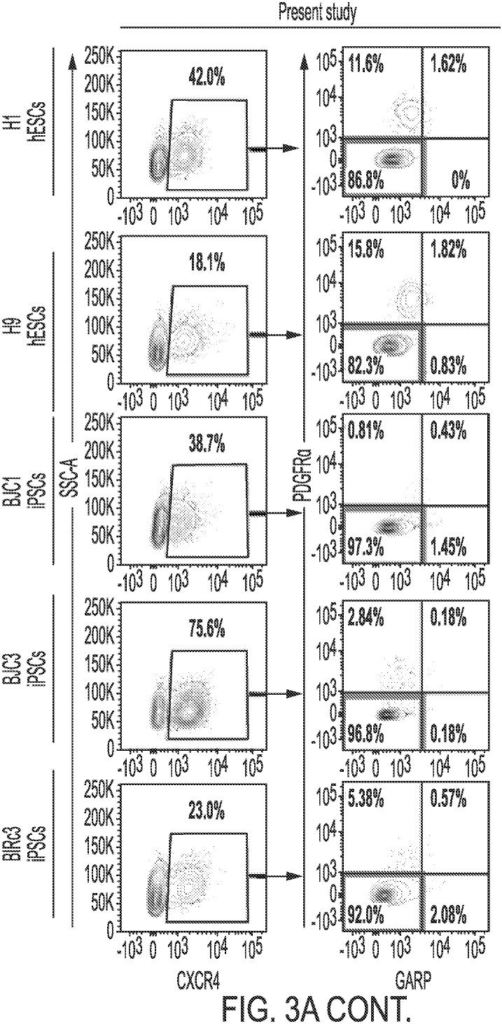

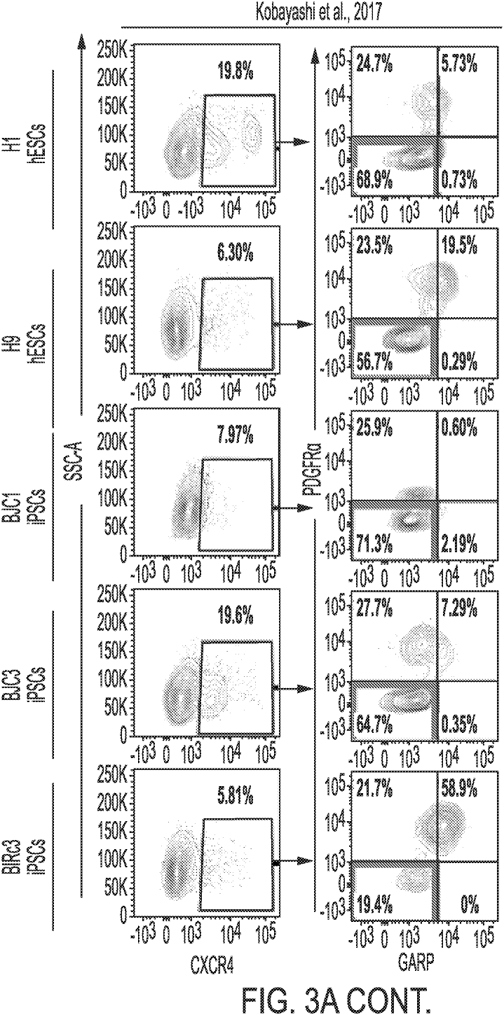

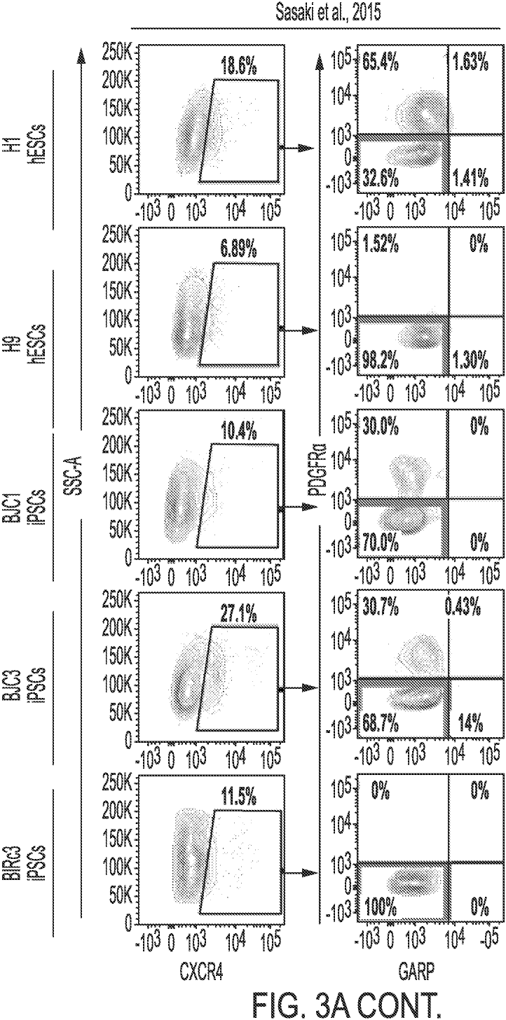

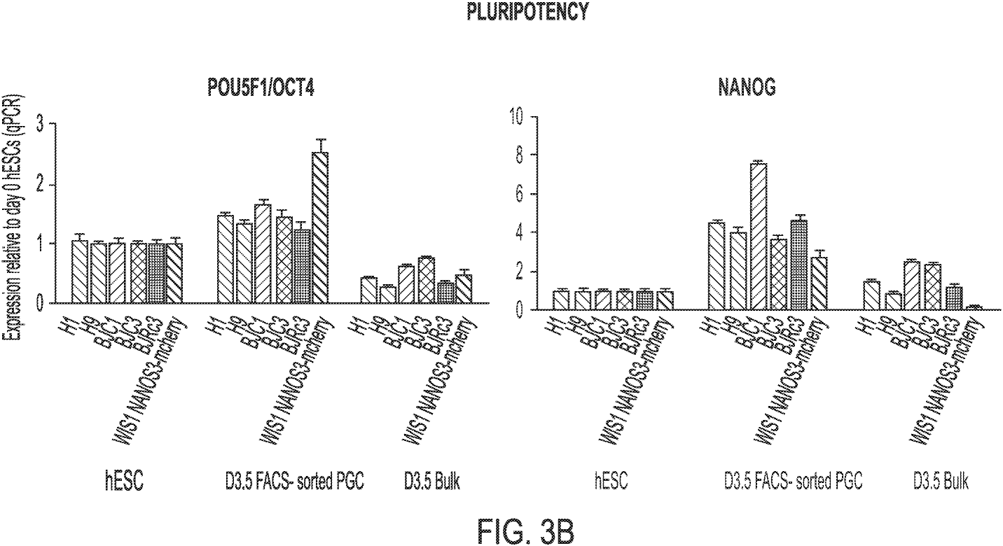

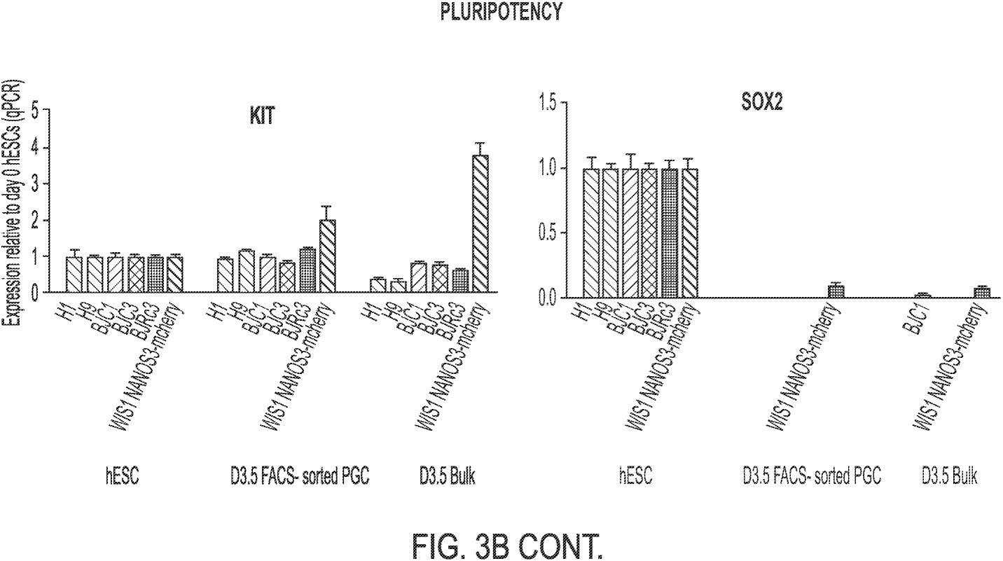

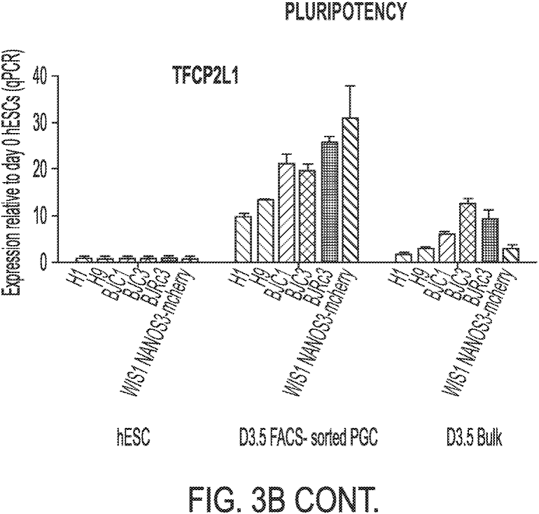

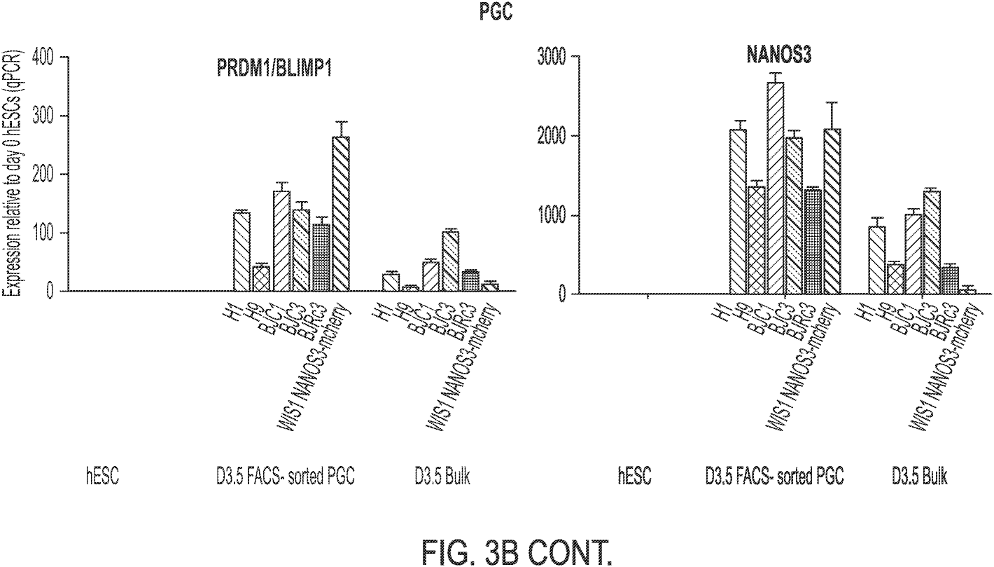

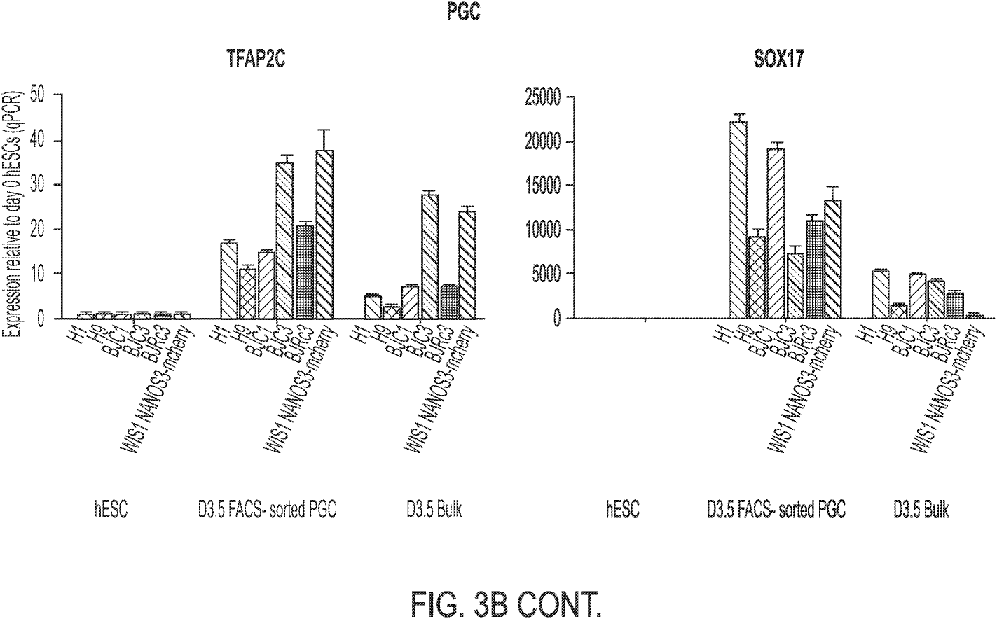

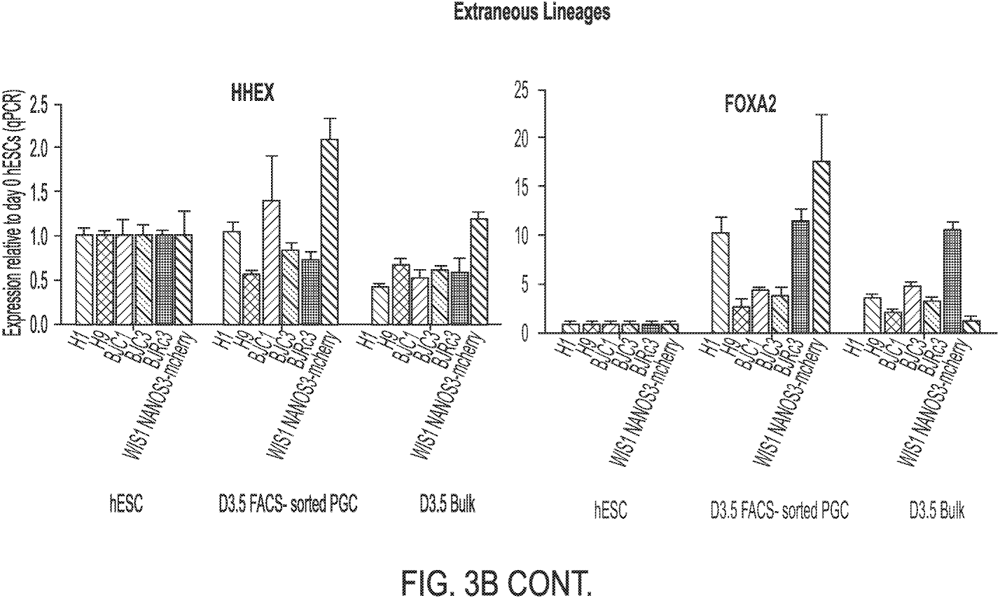

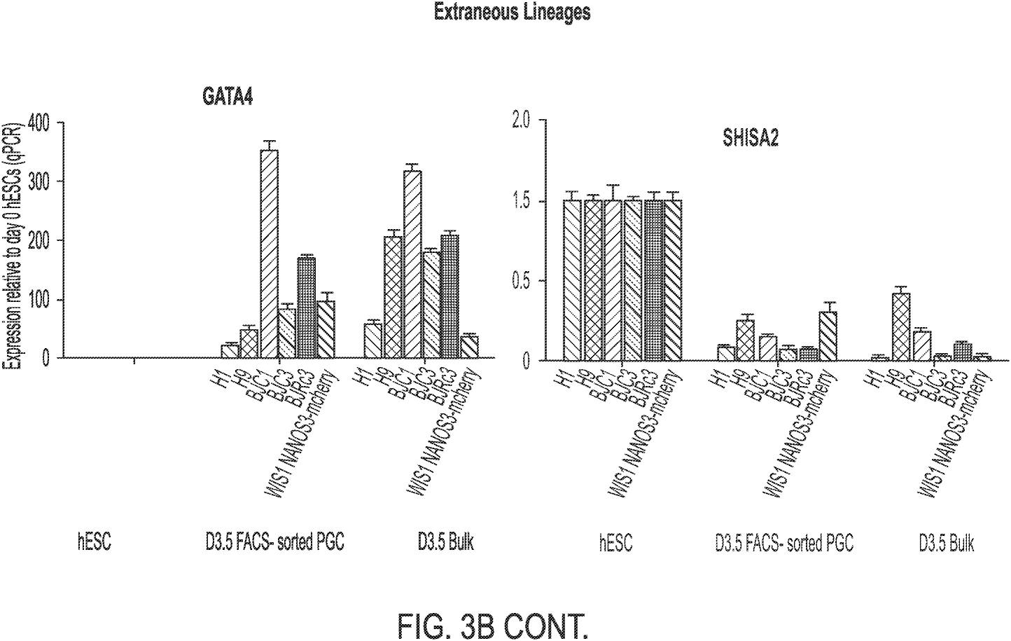

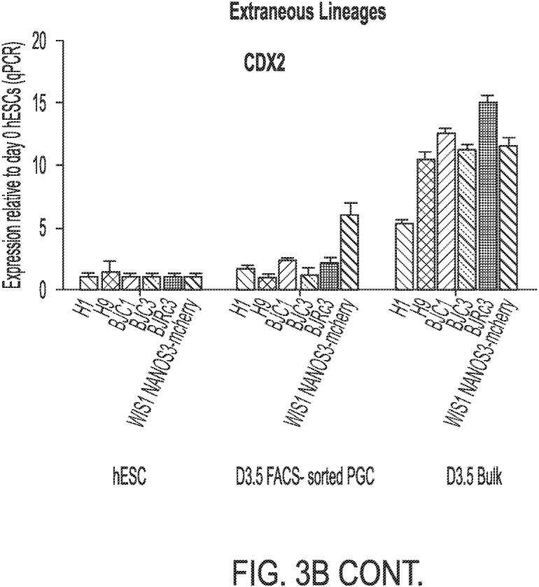

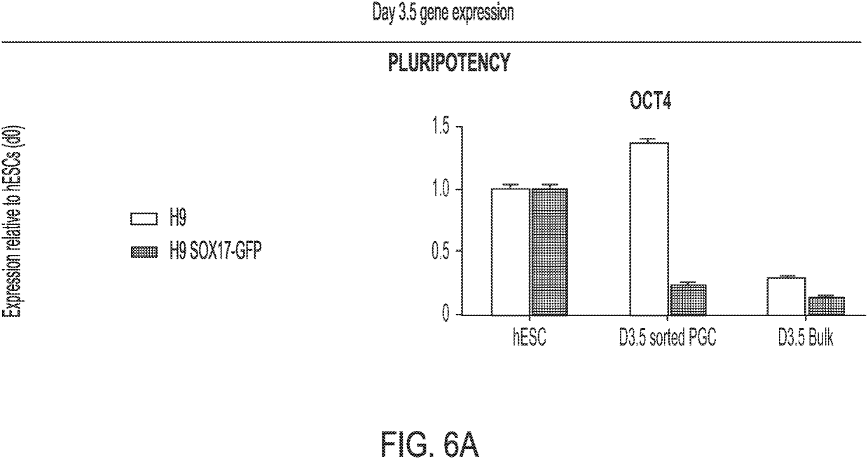

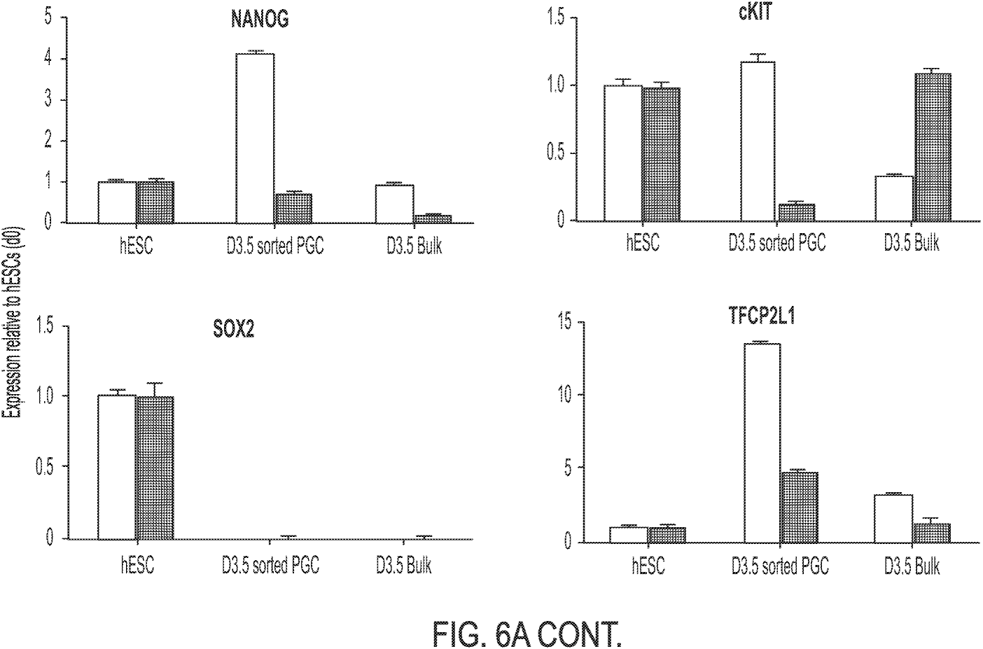

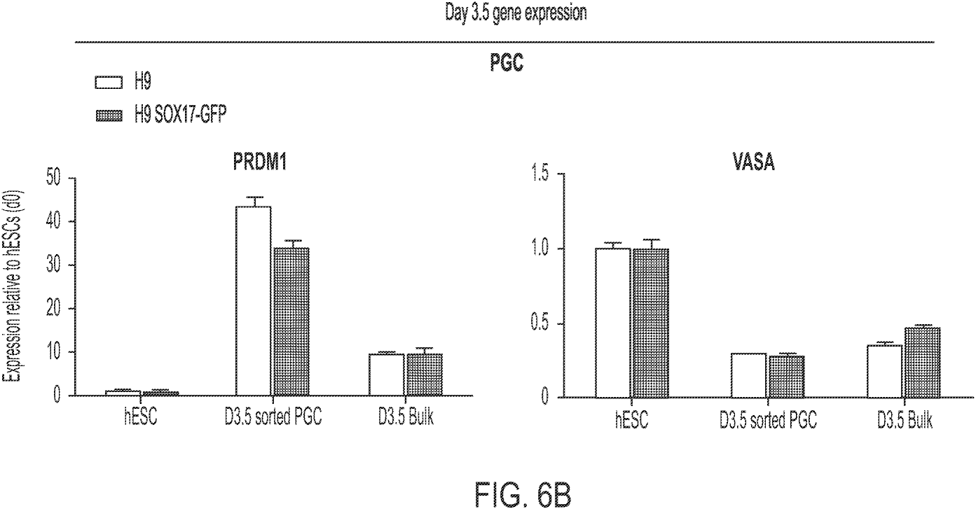

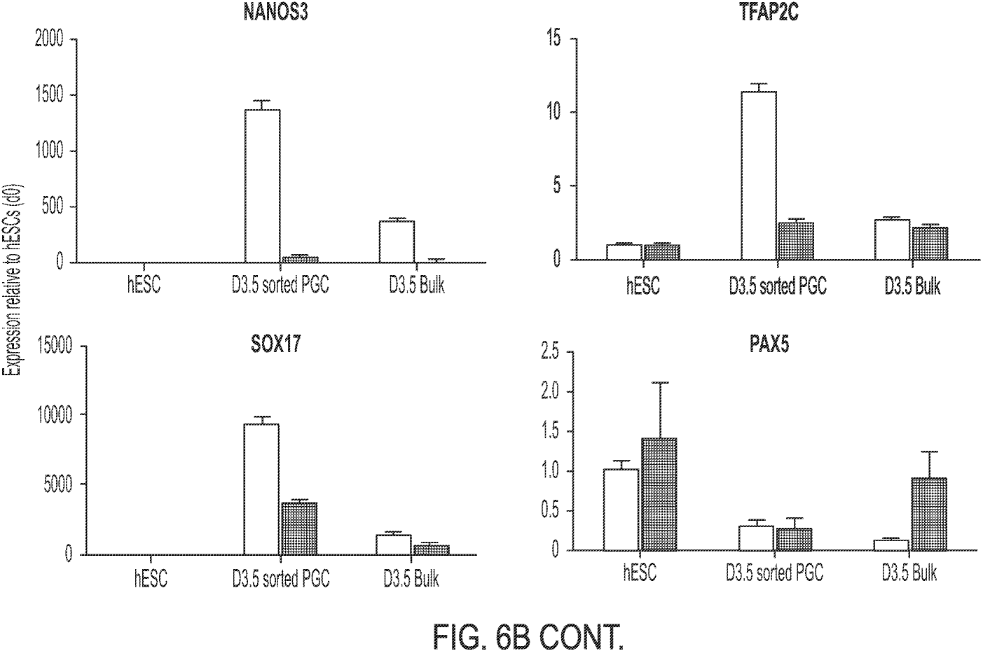

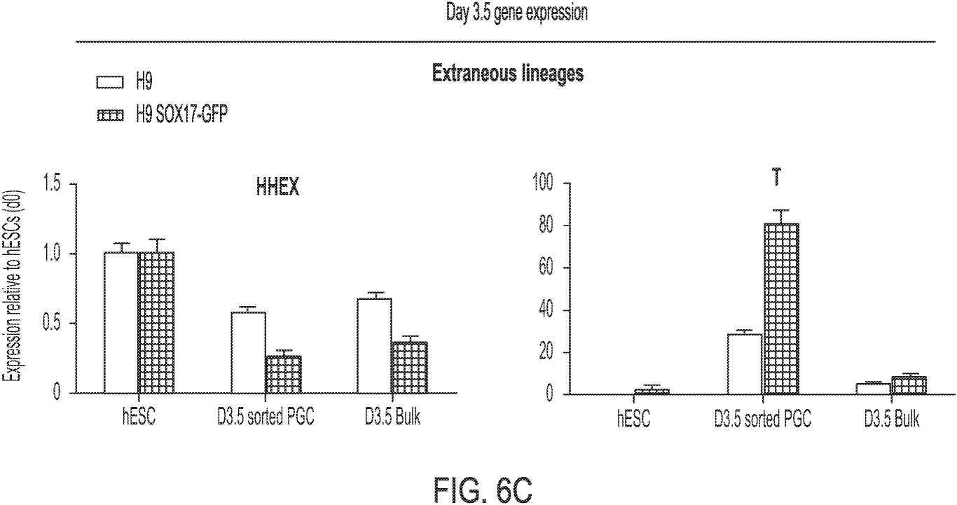

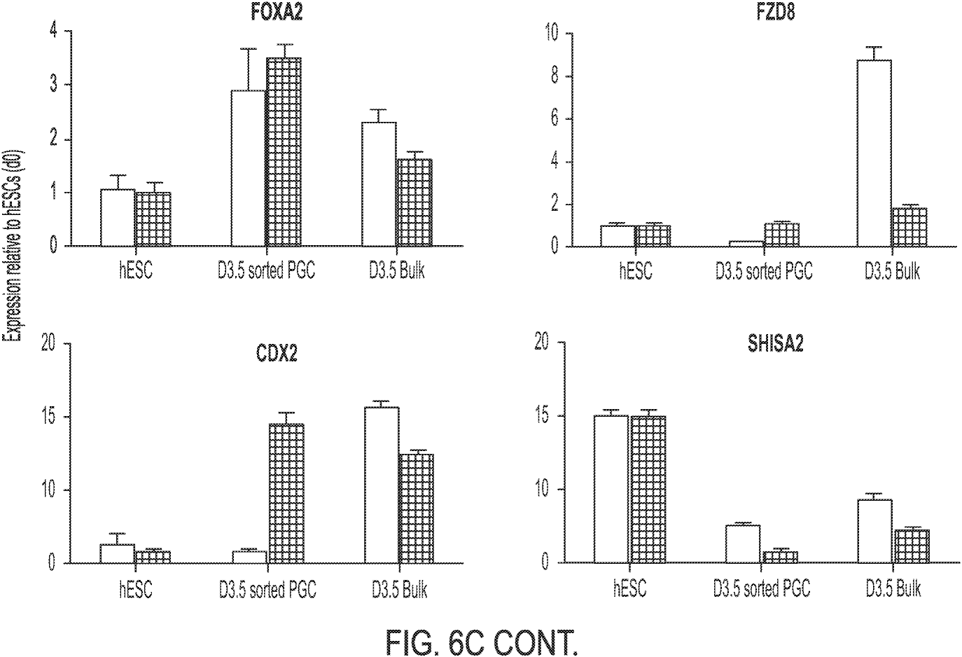

Combining these improvements together, we developed a monolayer, serum-free protocol (FIG. 1G) to consistently and reproducibly generate 20-30% pure NANOS3-mCherry⁺ PGCs within 3.5 days of in vitro differentiation (FIG. 1H). NANOS3-mCherry⁺ PGCs purified by fluorescence-activated cell sorting (FACS) expressed hallmark PGC markers, including POU5F1 (OCT4), NANOG, TFCP2L1, PRDMI (BLIMP1), NANOS3 and TFAP2C (AP2y) (FIG. 1i). hPSC-derived PGCs generated using our protocol did not express markers of endoderm (FOXA2, HHEX) or extraembryonic fate (CDX2), thus reaffirming their lineage specificity (FIG. 5G). At the protein level, PGCs showed co-expression of PGCs-specific markers like NANOG and PRDM1/BLIMP1 or SOX17 and PRDM1/BLIMP1 (FIG. 1K). When the same differentiation protocol was applied to the SOX17-GFP knock-in reporter hPSCs (Wang et al., 2011) we confirmed that the SOX17-GFP⁺ PGCs obtained by D3.5, expressed NANOS3 but not endodermal marker FOXA2(FIG. 5H), confirming that these PGCs were not SOX17⁺FOXA2⁺ endoderm (Loh et al., 2014). We ultimately applied our differentiation protocol across 6 different hESC/hiPSC lines and found that it reproducibly generated PGCs (detailed below; FIG. 3). However, we note that the SOX17-GFP reporter hPSCs lack one functional allele of SOX17, which is required for human PGC specification (Irie et al., 2014). Therefore SOX17-GFP⁺ PGCs generated from SOX17-GFP hPSCs had reduced expression of several PGC markers by comparison to wild-type hPSC-derived PGCs (FIGS. 6A-6C). This reveals potential disadvantages in using genetically-engineered reporter hPSC lines; below, we overcame this limitation by applying our differentiation protocol to wild-type hPSC lines.

Surface-marker profile of hPSC-derived PGCs: CXCR4⁺ PDGFRA⁻ GARP⁻. To produce and utilize human PGCs generated from wild-type hPSC lines, we require a means (e.g., cell-surface markers) to identify the successfully-generated PGCs without recourse to genetic reporters (Irie et al., 2014; Sasaki et al., 2015). Using our optimized monolayer platform for PGC differentiation, we thus sought to discover cell-surface markers in order to exclusively purify hPSC-derived PGCs from wild-type hPSC lines. Various markers such as alkaline phosphatase activity (Irie et al., 2014) and EpCAM and ITGA6 expression (Sasaki et al., 2015) have been reported to enrich for PGCs, but are not specific to PGCs, as they also mark undifferentiated hPSCs.

We robotically screened the expression of 369 cell-surface markers using high-throughput FACS (Loh et al., 2016) on SOX17-GFP hPSCs that had been differentiated towards PGCs for 3.5 days. We assessed surface marker expression on D3.5 SOX17-GFP⁺ PGCs vs. SOX17-GFP⁻ non-PGCs; undifferentiated hPSCs were also included as a negative control (Table 5). This confirmed that EpCAM and ITGA6 (Sasaki et al., 2015) were not specific markers; they were both expressed on hPSCs as well as PGCs (Table 5).

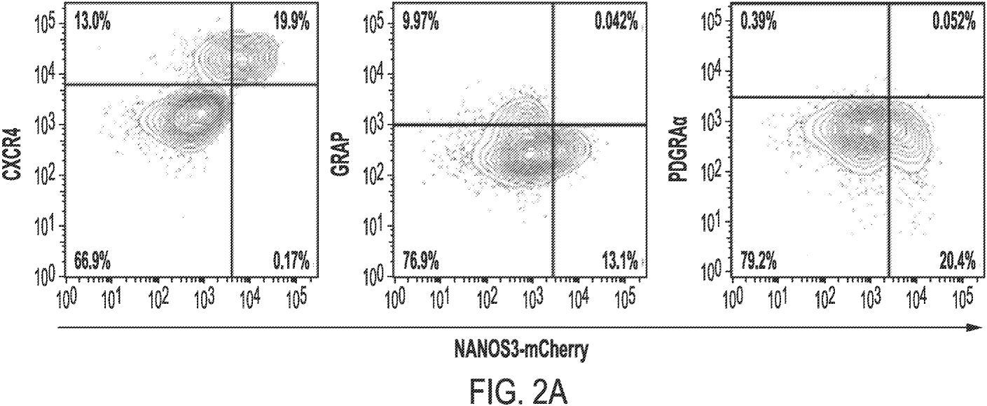

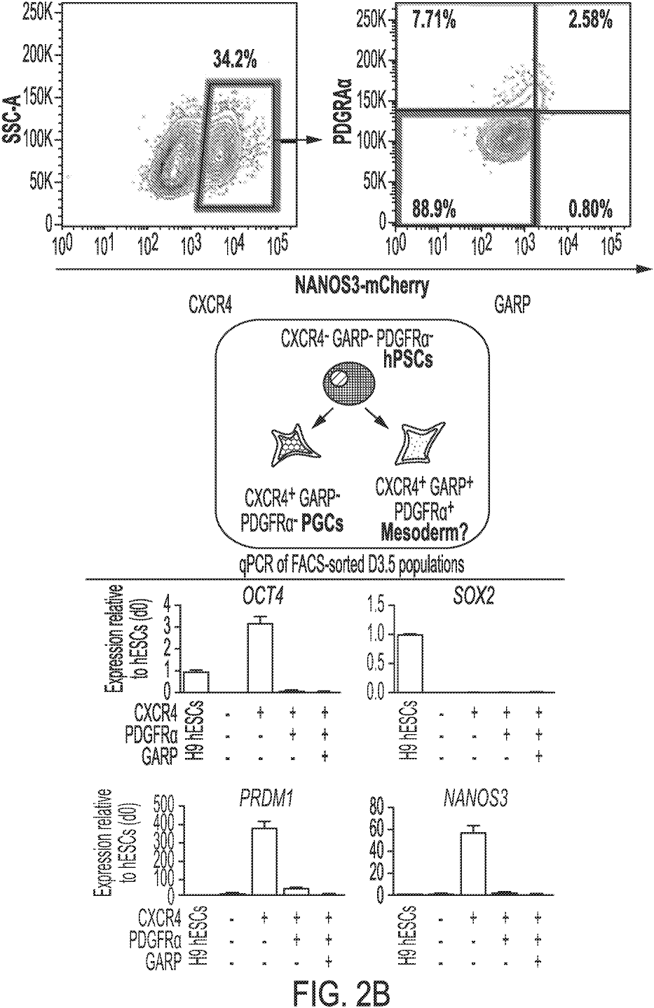

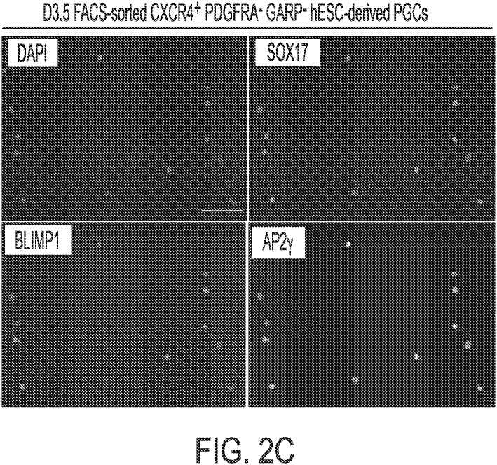

In our analysis, the most specific positive marker for SOX17-GFP⁺ PGCs was the chemokine receptor CXCR4/CD184 (Table 5), which we validated to similarly mark NANOS3-mCherry⁺ PGCs (FIG. 2A). In model organisms, Cxcr4 is known to be expressed by later-stage PGCs, where it enables their migration towards the gonads in response to Cxcl12 (Molyneaux et al., 2003); this may also be conserved in human (Mitsunaga et al., 2017). However, CXCR4 is also expressed on mesodermal derivatives (McGrath et al., 1999), and therefore negative expression of mesodermal markers is necessary to exclude mesoderm. We found that the mesodermal markers PDGFRA/CD140A (Kattman et al., 2011) and GARP/LRRC32 (Loh et al., 2016) were expressed on the D3.5 non-PGCs (Table 5, FIG. 2A), thus providing a means to eliminate mesoderm.

Taken together, by relying on a combination of positive (CXCR4) and negative (PDGFRA, GARP) markers, we defined a CXCR4⁺ PDGFRA⁻ GARP⁻ surface marker profile for hPSC-derived PGCs. In differentiated D3.5 cultures, the CXCR4⁺ PDGFRA⁻ GARP⁻ fraction contained all PGCs; other combinations of these surface markers did not enrich for PGCs (FIG. 2B). Immunostaining of FACS-sorted hPSC-derived D3.5 CXCR4⁺ PDGFRA⁻ GARP⁻ cells confirmed that they ubiquitously expressed the PGC marker proteins SOX17, PRDM1/BLIMP1 and TFAP2C/TFAP2y (FIG. 2B).

PGCs can be consistently generated across diverse hESC and hiPSC lines. Our monolayer differentiation method was more efficient at generating PGCs by comparison to two prevailing 3D-based methods (Kobayashi et al., 2017; Sasaki et al., 2015) (FIG. 3A). Across a panel of 5 distinct, wild-type male and female hESC and hiPSC lines, the present differentiation method generated an average of 36.7±22.6% (with peaks as high as 73.2%) pure CXCR4⁺ PDGFRA⁻ GARP⁻ PGCs, which was several-fold more efficient than previously possible with prior methods (7.3±5.6% (Kobayashi et al., 2017) and 10.0±5.2% (Sasaki et al., 2015)) in side-by-side comparisons (FIG. 3A). Therefore, despite the tremendous enthusiasm in differentiating 3D aggregates or “organoids” (Lancaster and Knoblich, 2014), 2D cultures may offer certain advantages with regard to the efficiency and simplicity of differentiation.

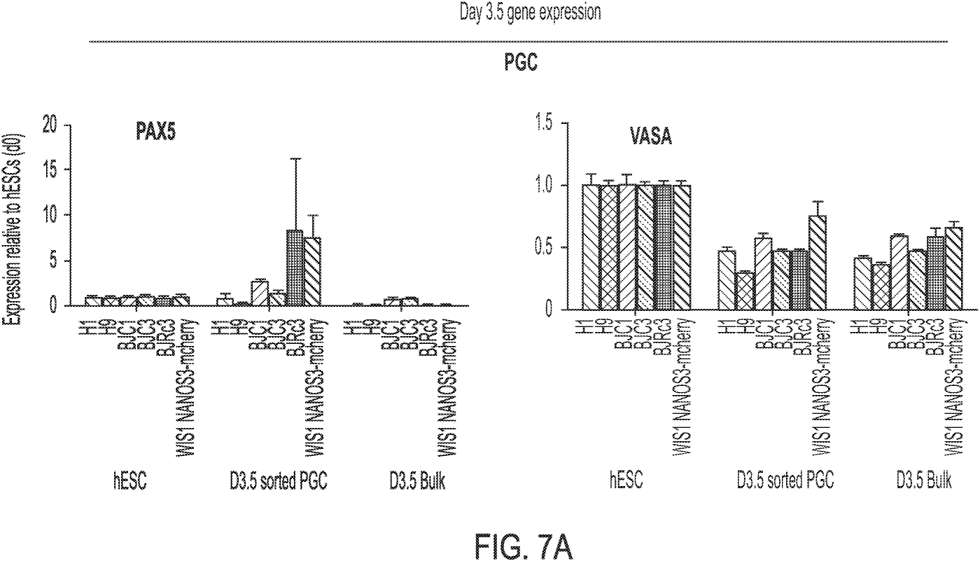

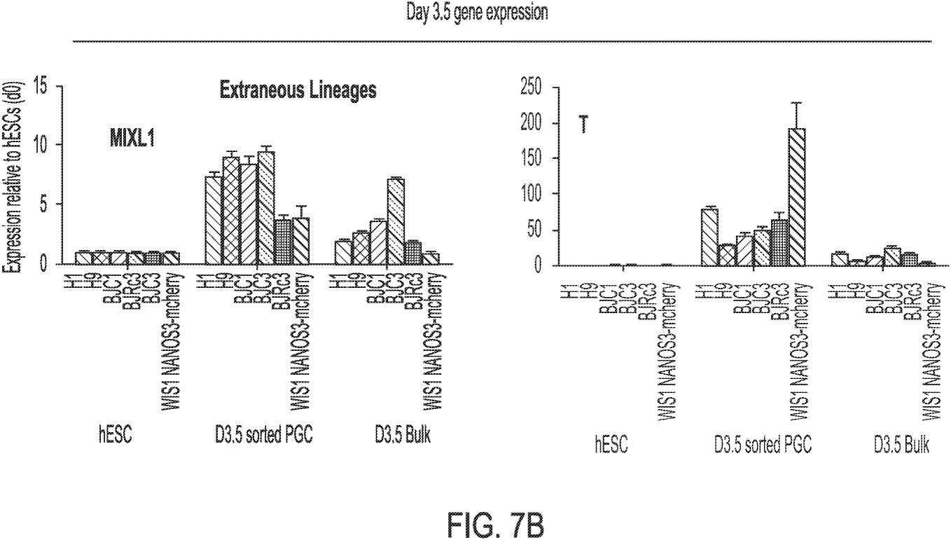

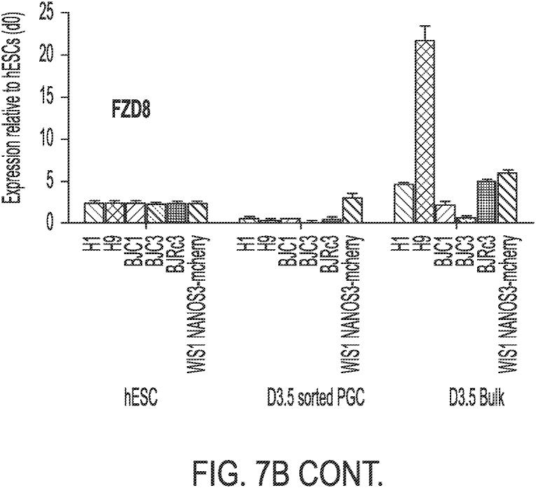

Our CXCR4⁺ PDGFRA⁻ GARP⁻ cell-surface marker signature allowed us to purify differentiated PGCs across all hESC and hiPSC lines tested, using our improved differentiation strategy and without recourse to transgenic reporters (FIG. 3B, FIGS. 7A-7B). Across all lines, hPSC-derived CXCR4⁺ PDGFRA⁻ GARP⁻ PGCs demonstrated upregulation of hallmark PGC markers without substantial expression of endodermal or mesodermal markers (FIG. 3B, FIGS. 7A-7B). This exemplifies the fidelity of PGC specification across distinct genetic backgrounds and also demonstrates the utility of the surface-marker profile.

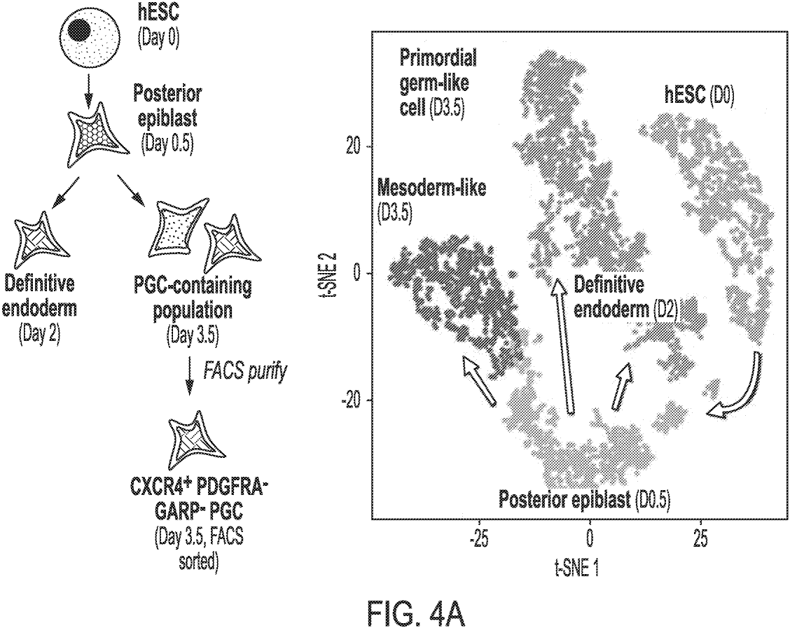

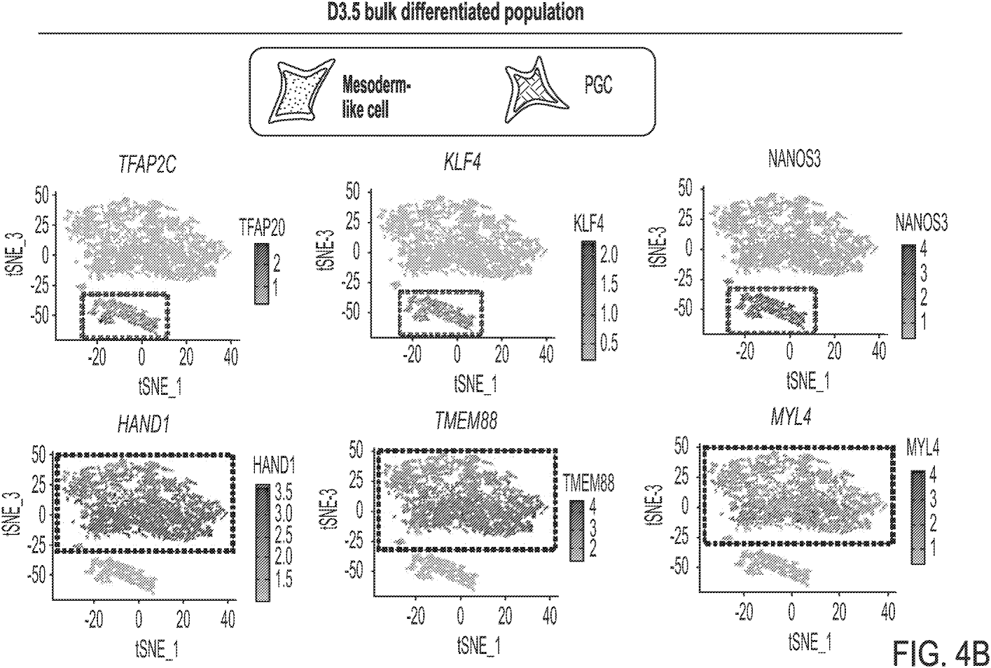

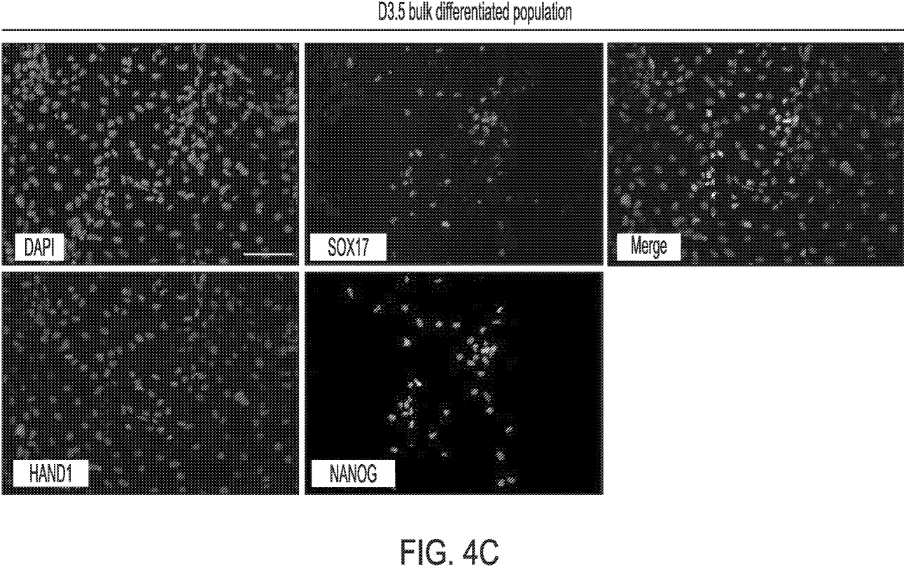

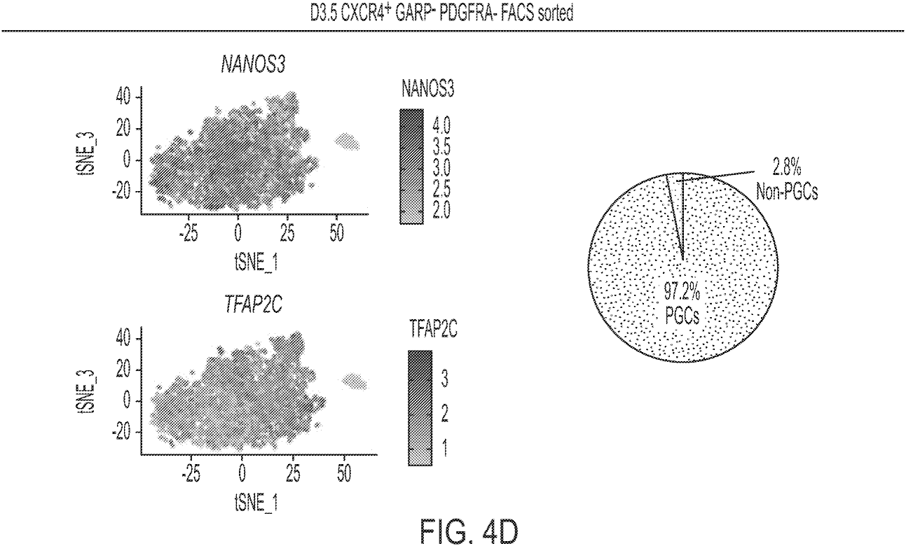

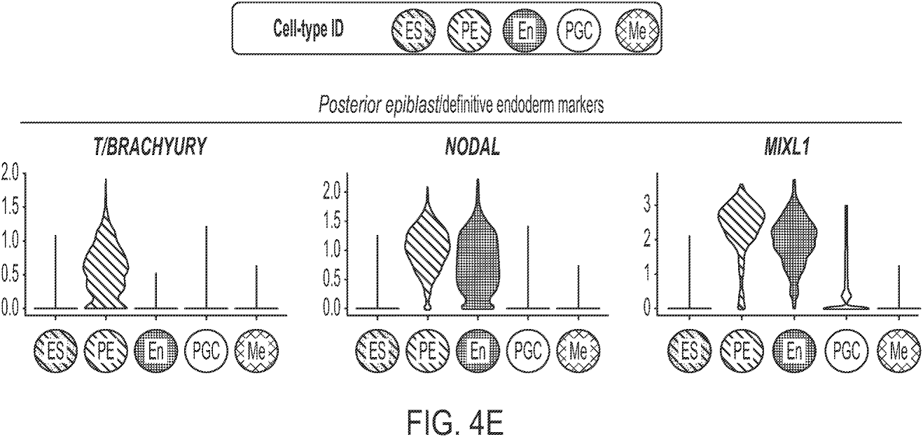

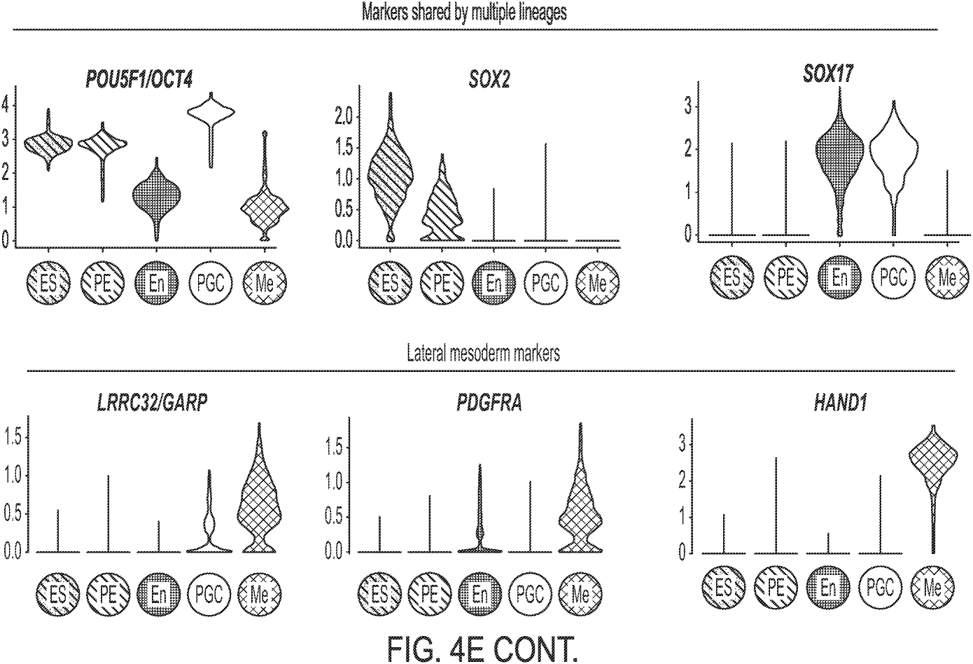

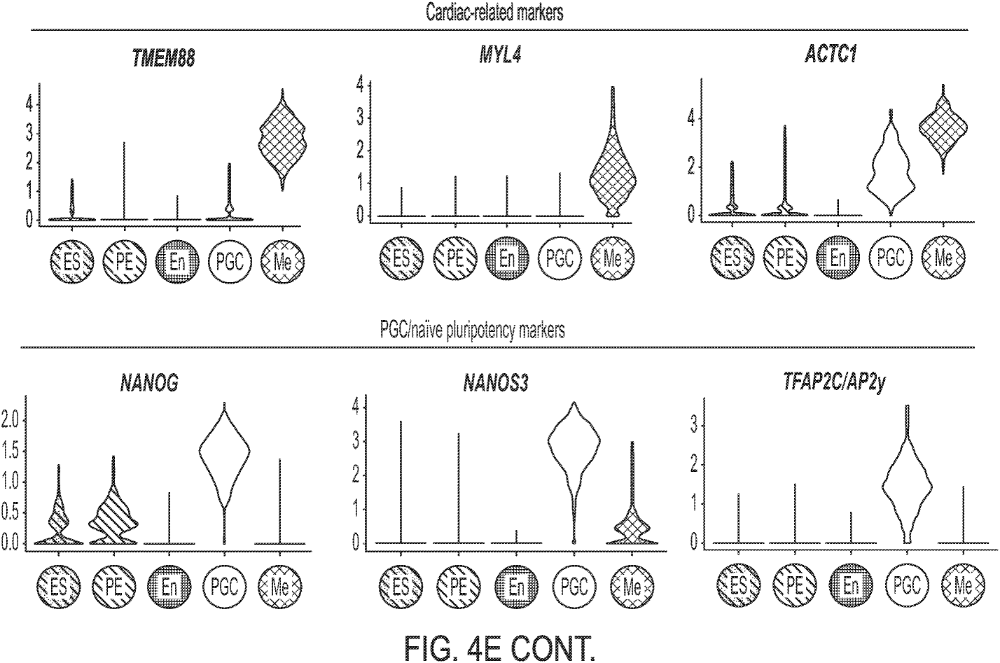

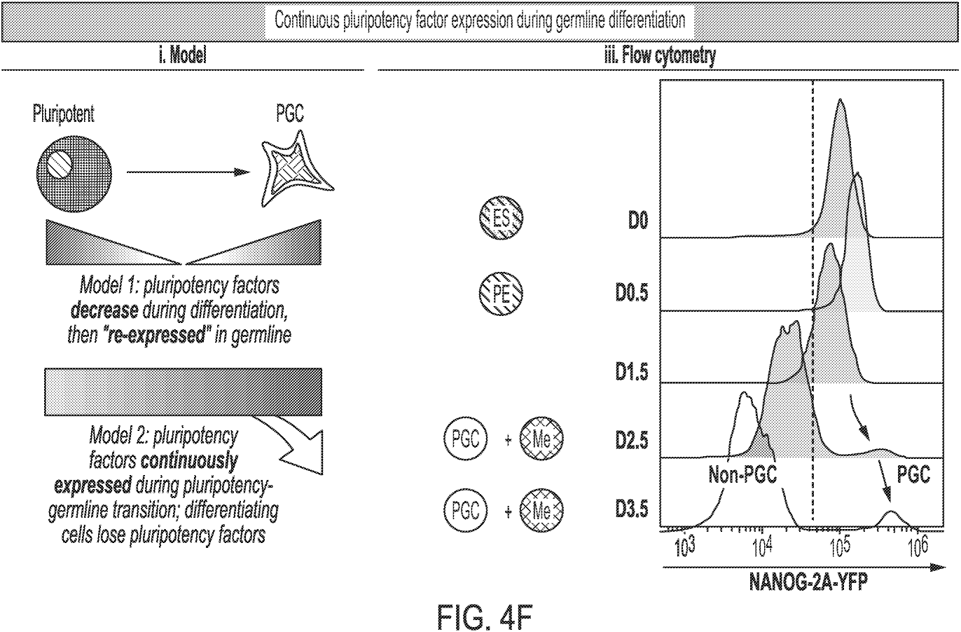

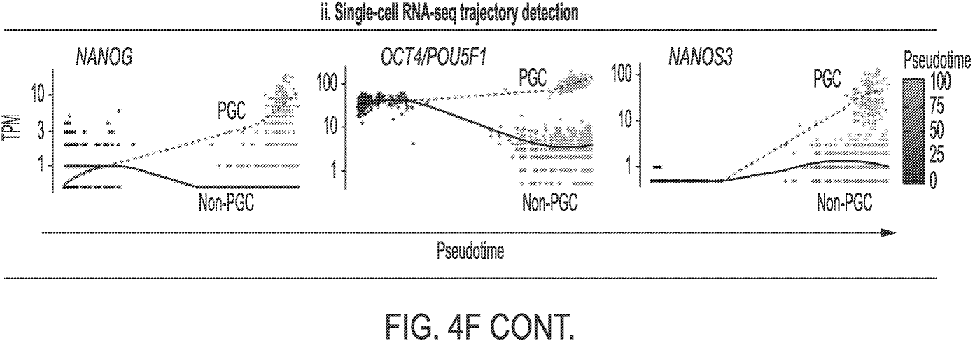

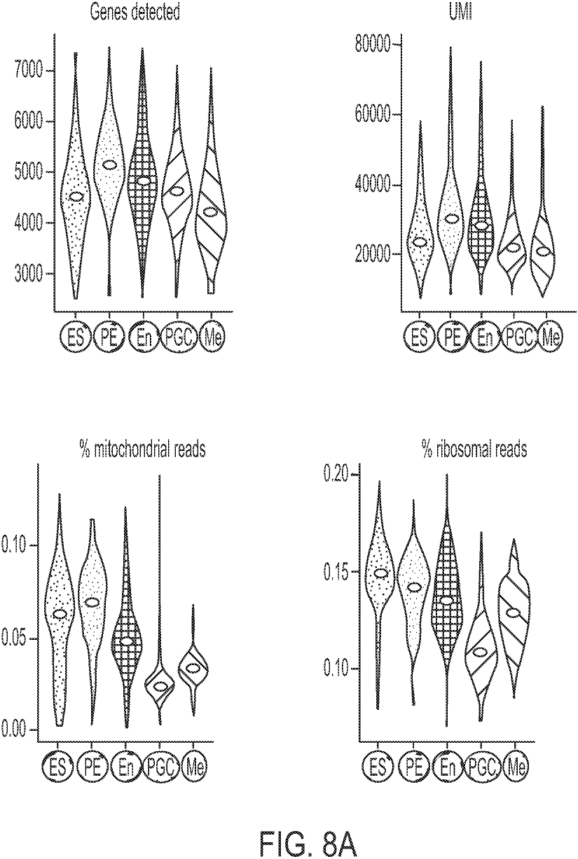

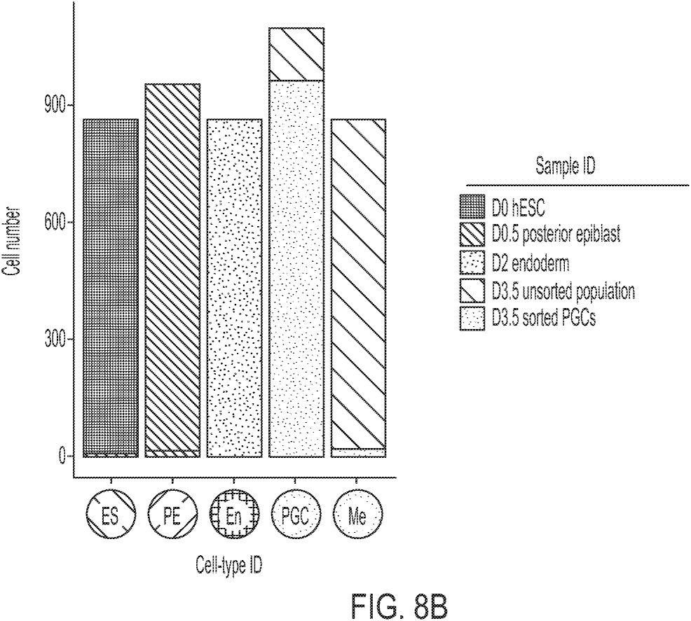

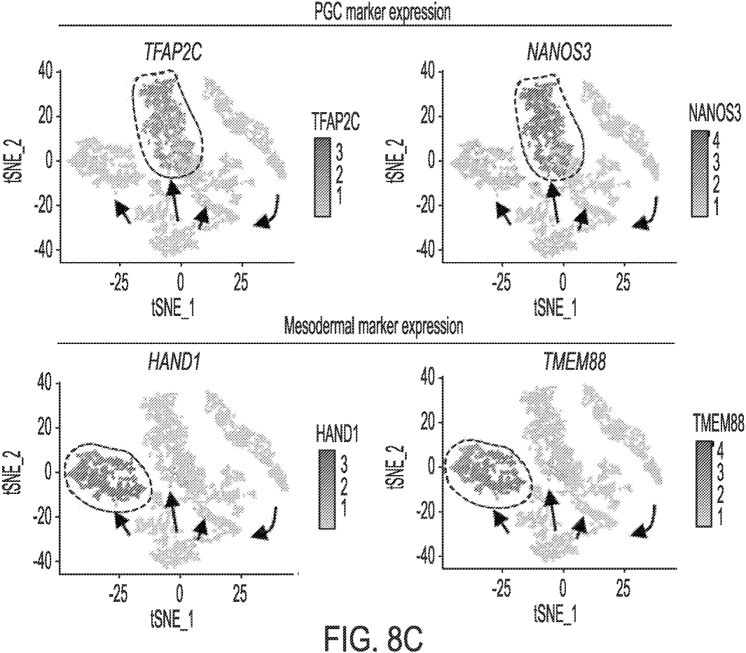

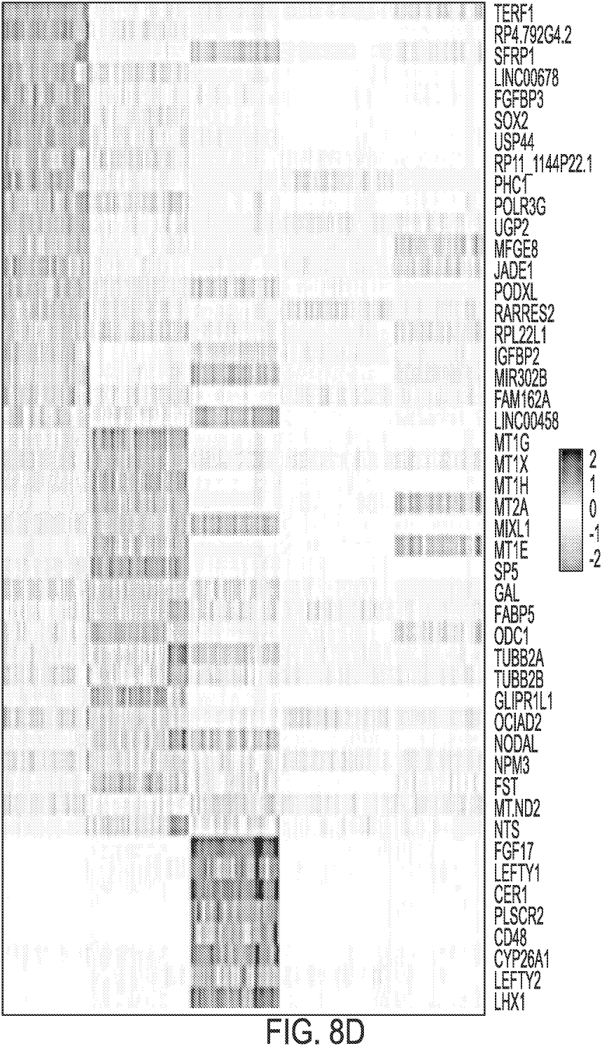

Tracking the trajectory and uniformity of PGC specification in vitro using single-cell RNA-sequencing. Finally, we illuminated the stepwise changes in gene expression as hPSCs incipiently differentiated into posterior epiblast (D0.5) and then into a PGC-containing population (D3.5) by performing single-cell RNA sequencing (scRNAseq) of all of these populations (FIG. 4A, FIG. 8A). Given that we showed above that the D3.5 population is heterogeneous (FIGS. 1-3), scRNAseq was important to detail the cellular diversity of this population and to obtain a refined transcriptional signature only of the PGCs. We also performed scRNAseq of D2 definitive endoderm (Loh et al., 2014)—a lineage derived from the PS (and thus, on a related but distinct lineage path from PGCs)-to clarify the relationship between human PGCs and endoderm, given that human PGCs have been previously reported to express “endodermal” markers SOX17 and GATA4 (Irie et al., 2014; Sasaki et al., 2015). Taken together, we analyzed 24,473 cells by scRNAseq, with a median of >4000 genes detected per cell in each cell population (FIG. 8A).

scRNAseq showed that the D3.5 bulk differentiated population was transcriptionally heterogeneous, comprising two distinct subsets (FIG. 4B, FIG. 8B). One subset comprised PGCs expressing NANOS3, TFAP2C and KLF4 (FIG. 4B, FIG. 8C). Intriguingly, the non-PGCs expressed lateral mesoderm marker HAND1 and the cardiac mesoderm markers TMEM88, MYOSIN LIGHT CHAIN 4 (MYL4) and ALPHA CARDIAC ACTIN (ACTCI) (Loh et al., 2016; Novikov and Evans, 2013) (FIG. 4B, FIGS. 8C, 8D). This suggests that the “mis-differentiated”, non-PGCs at D3.5 are mesoderm-like cells, as evinced by HAND1 protein expression in the D3.5 non-PGCs (FIG. 4C). Indeed, the principal signals we used to differentiate posterior epiblast into PGCs (BMP activation and WNT inhibition) are the same ones that differentiate primitive streak into cardiac mesoderm (Loh et al., 2016). However, FACS sorting of the D3.5 population for CXCR4⁺ GARP⁻ PDGFRA⁻ cells (as described in FIG. 3) yielded an essentially homogeneous (97.2%) population of PGCs, as shown by scRNAseq of the FACS-sorted cells (FIG. 4D). This thus reaffirms the power of our cell-surface marker profile to precisely isolate PGCs from a heterogeneous cell population.

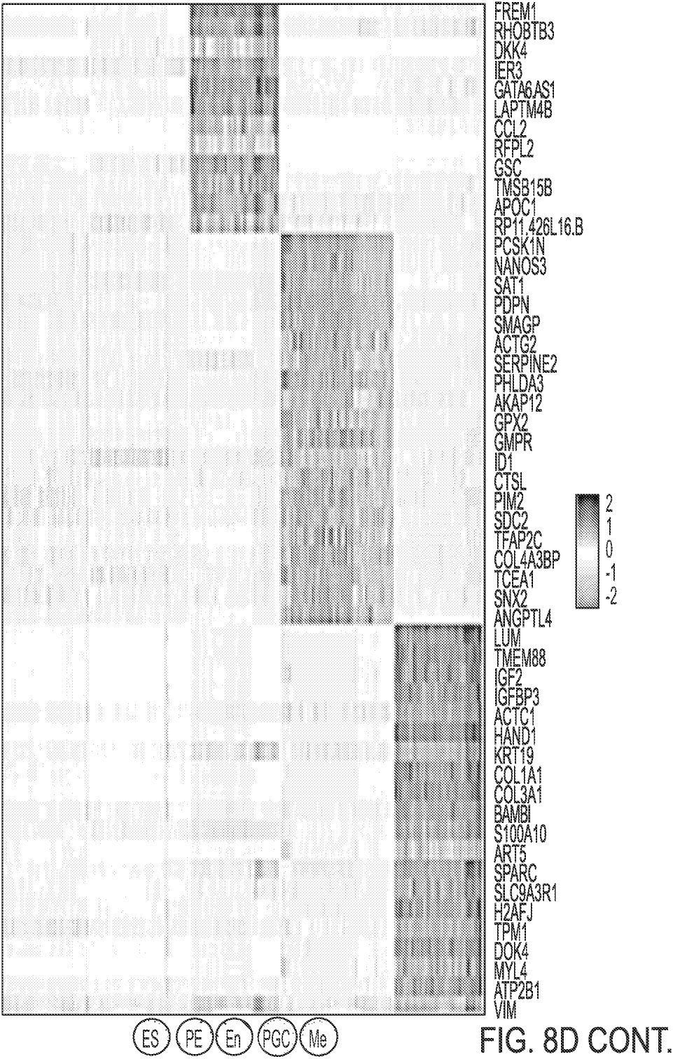

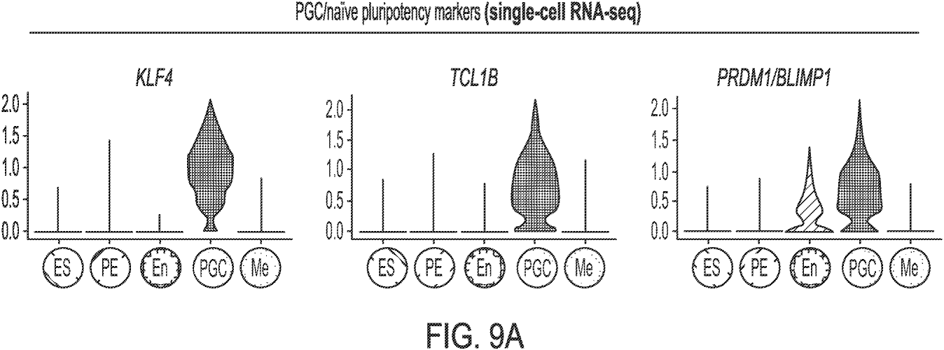

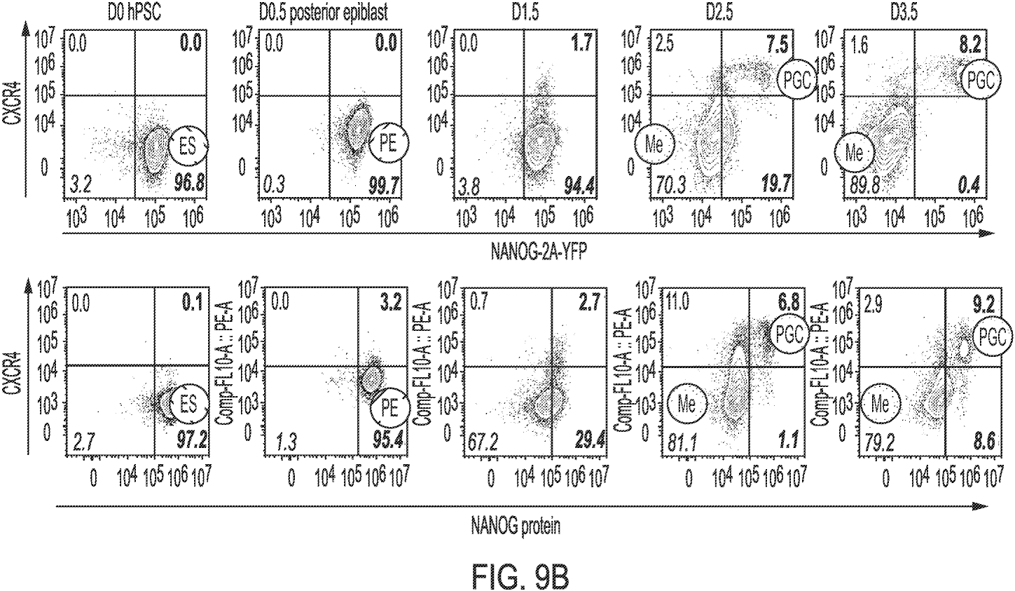

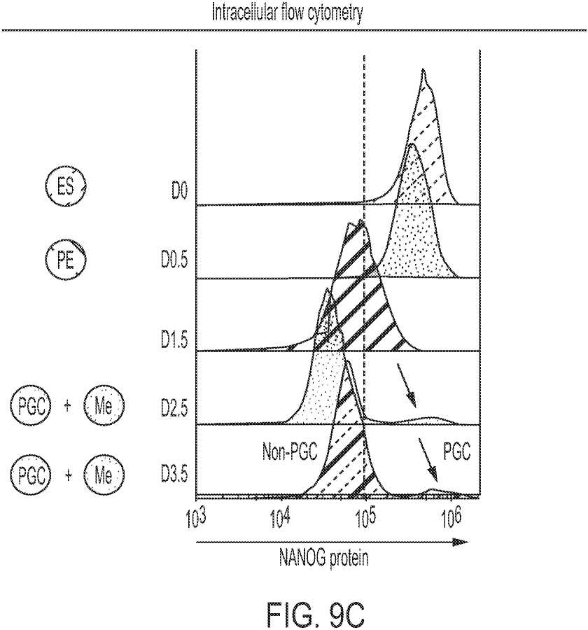

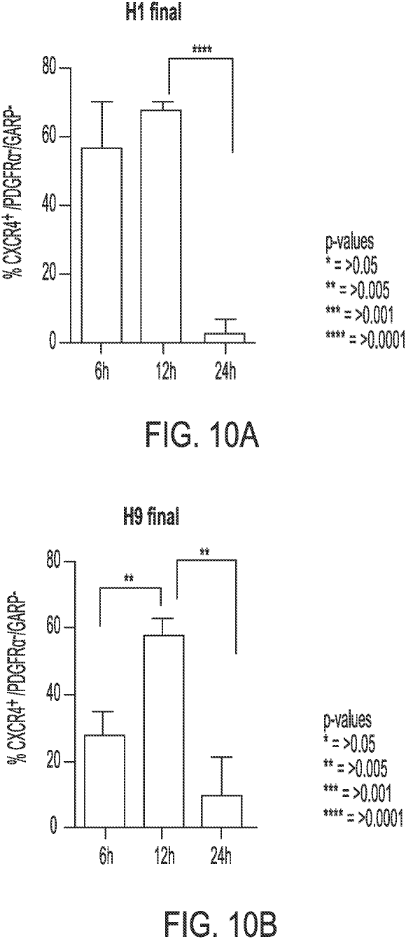

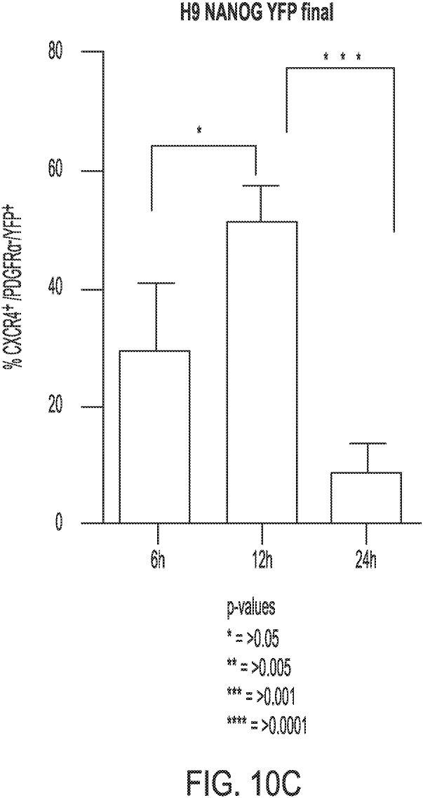

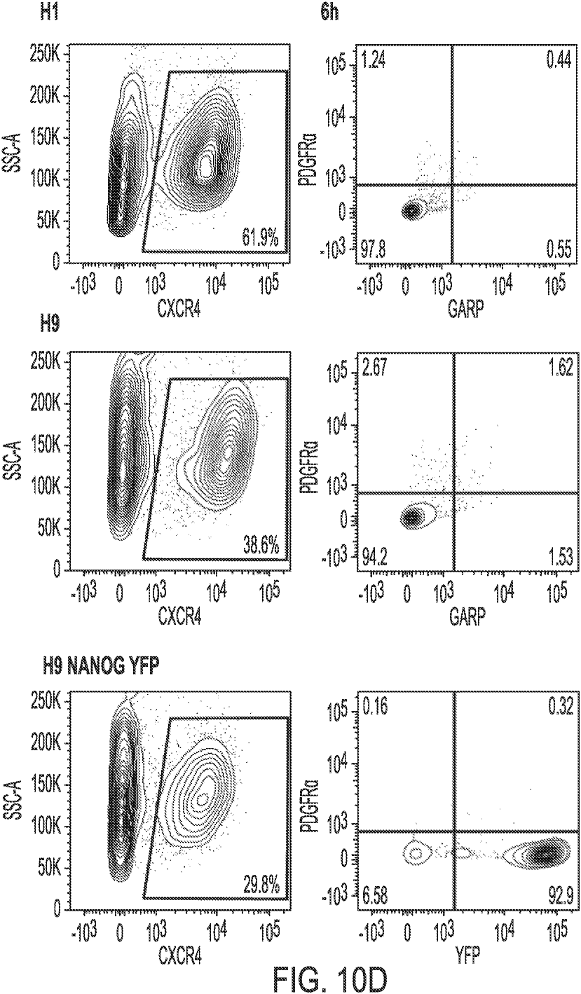

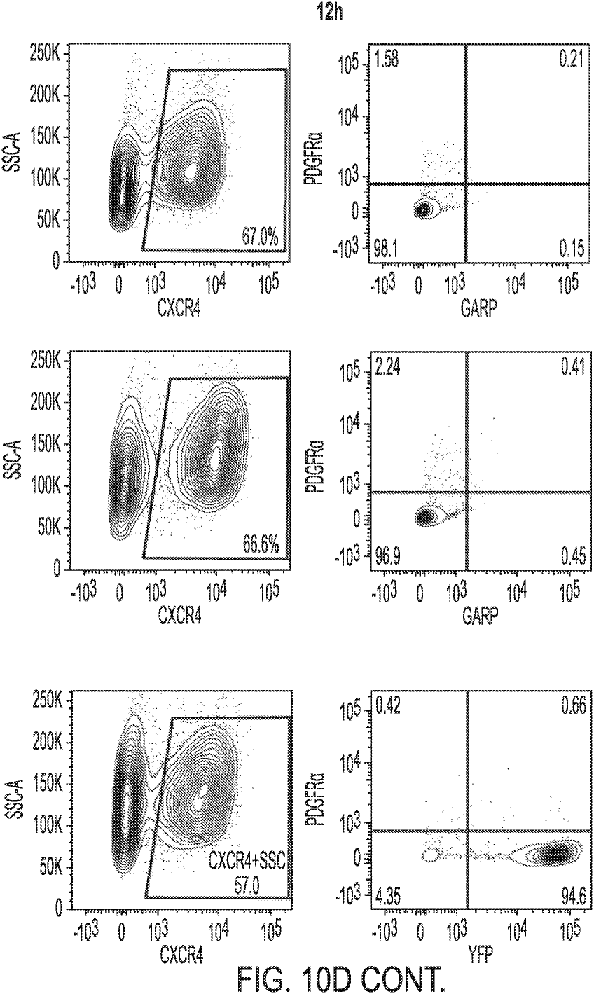

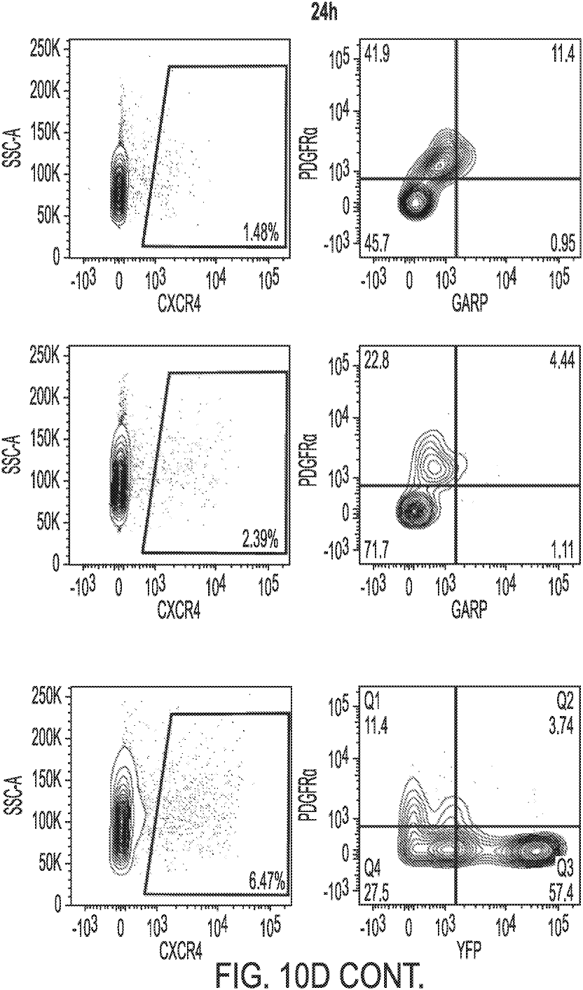

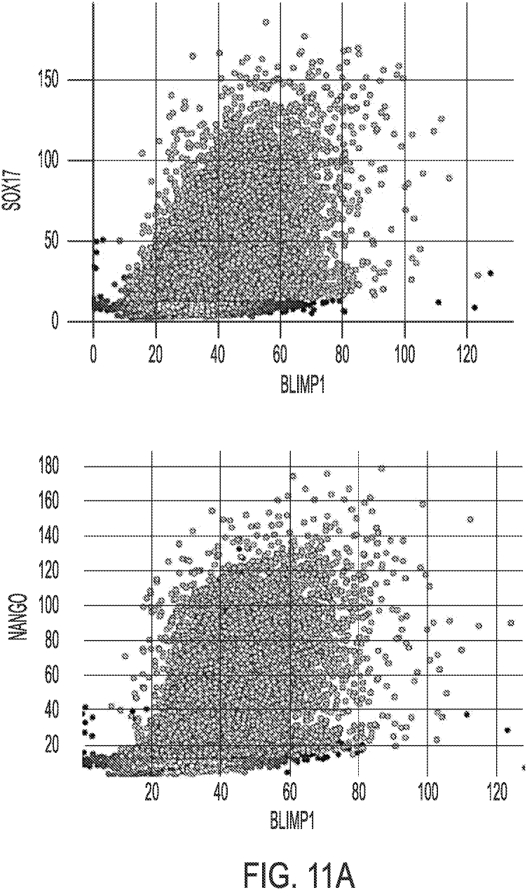

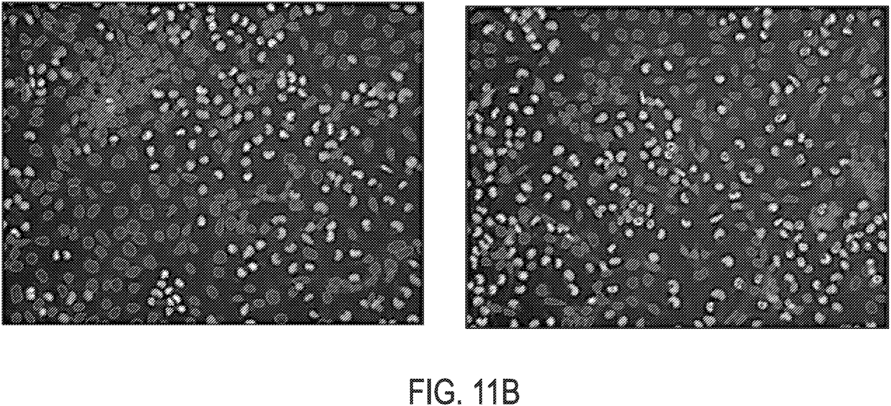

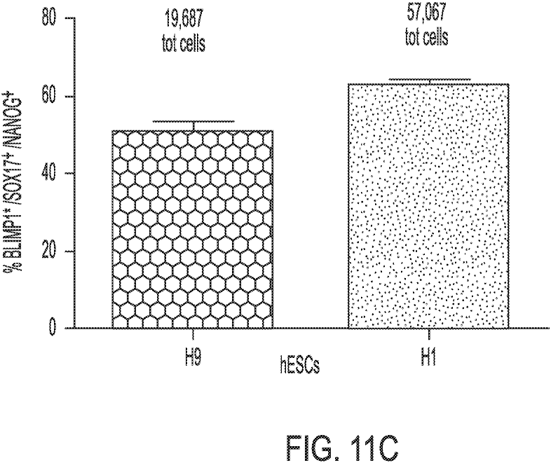

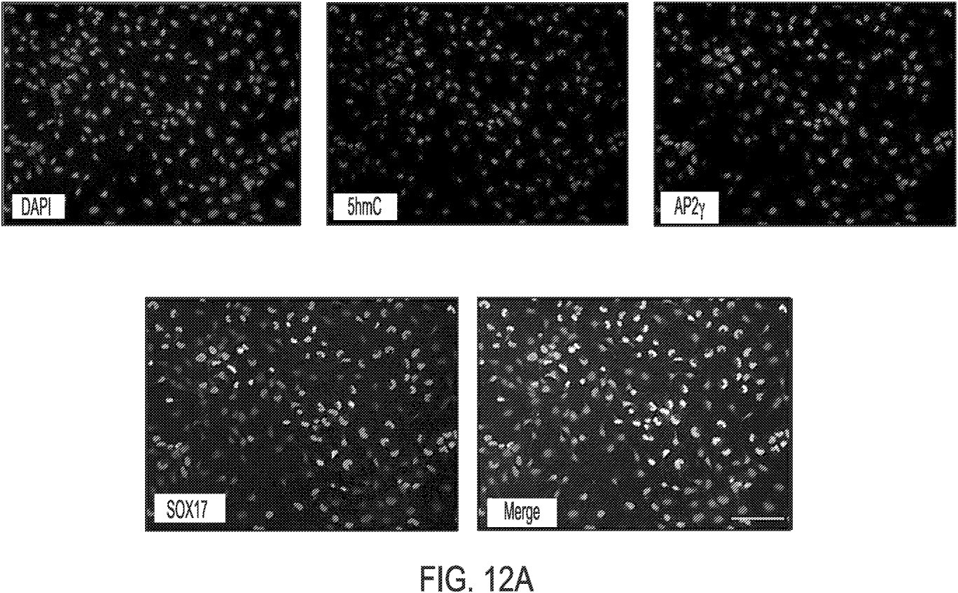

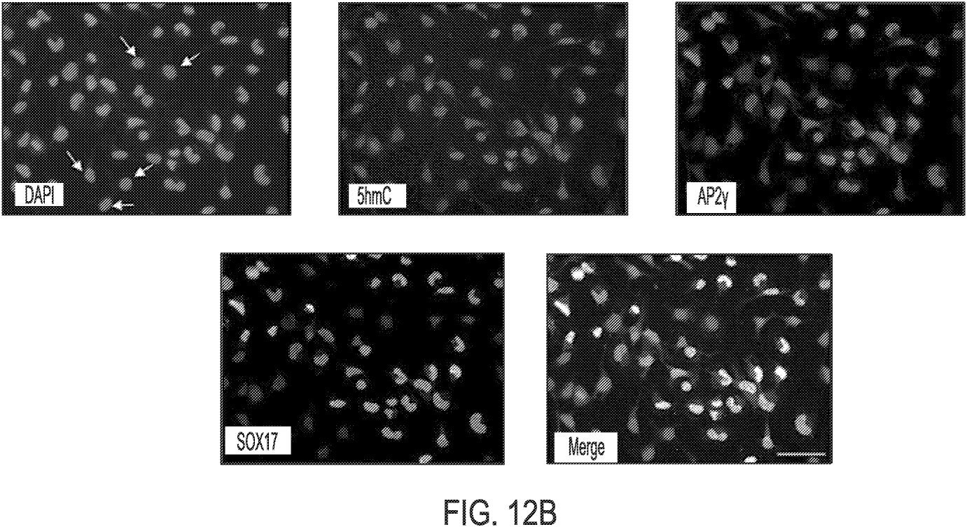

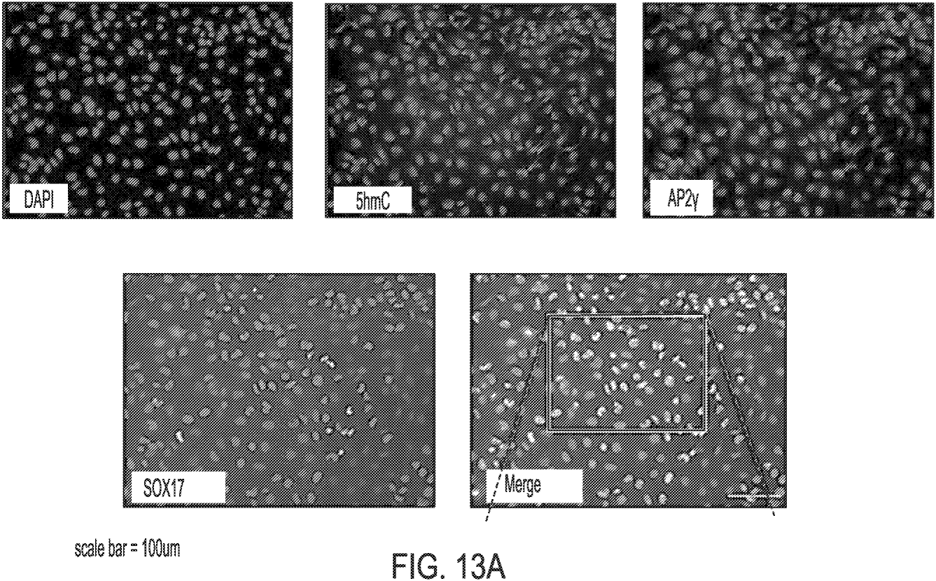

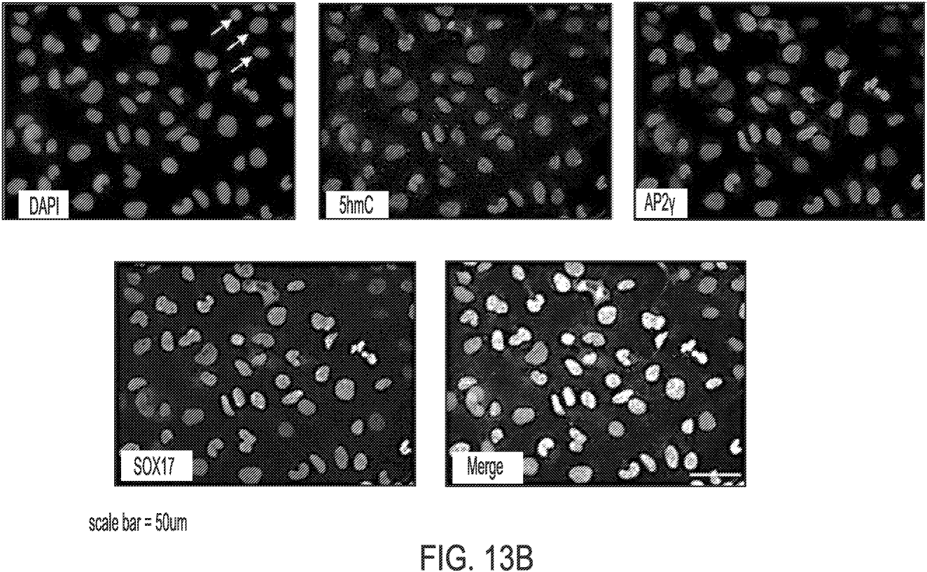

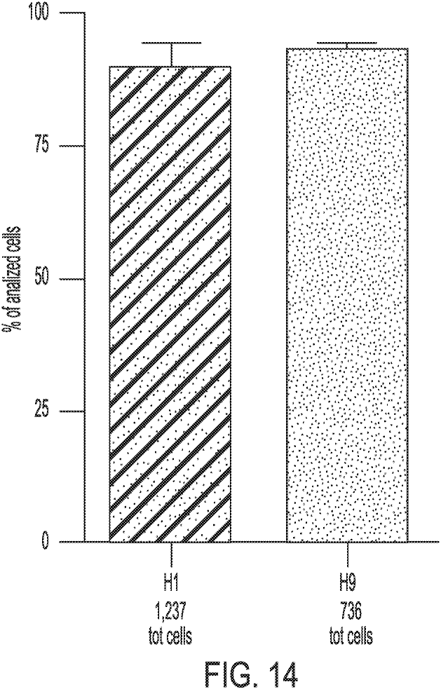

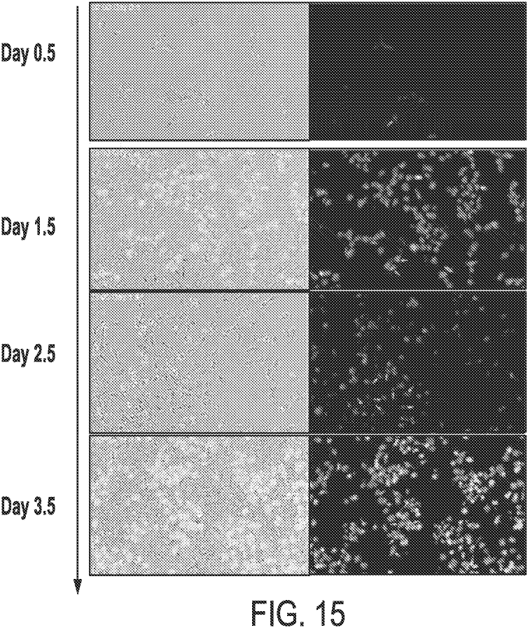

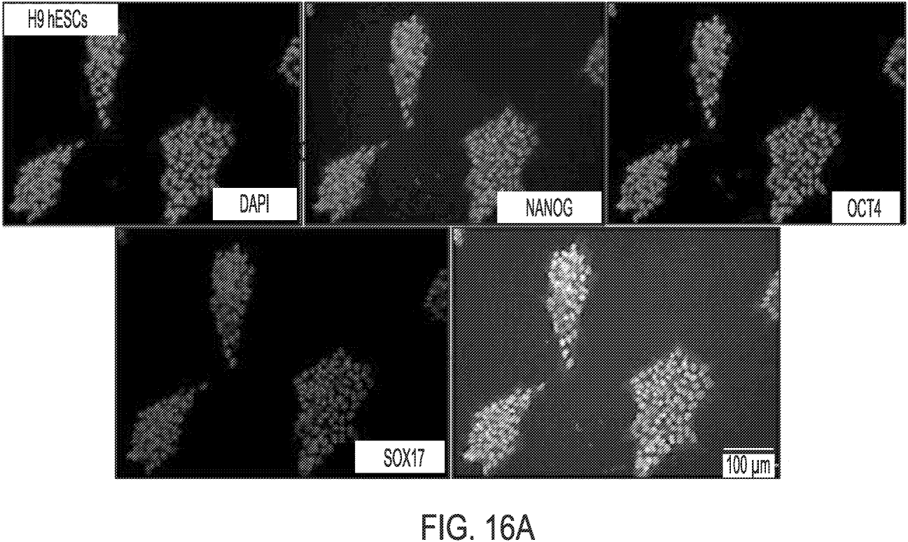

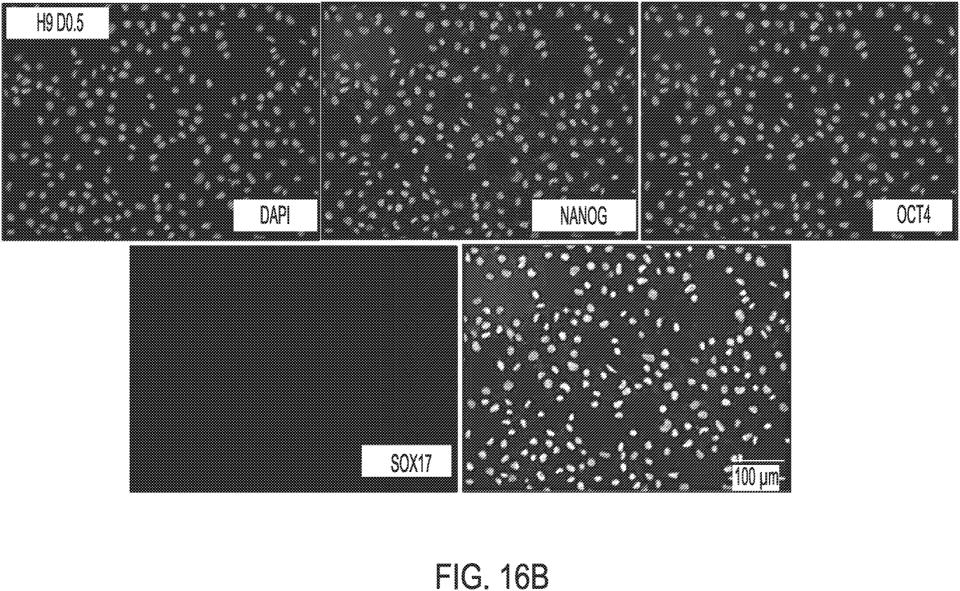

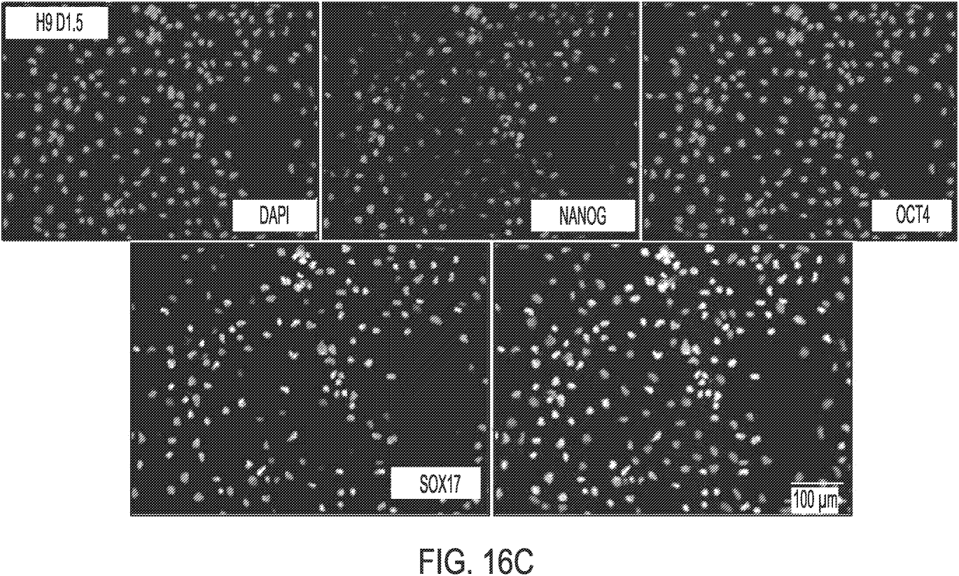

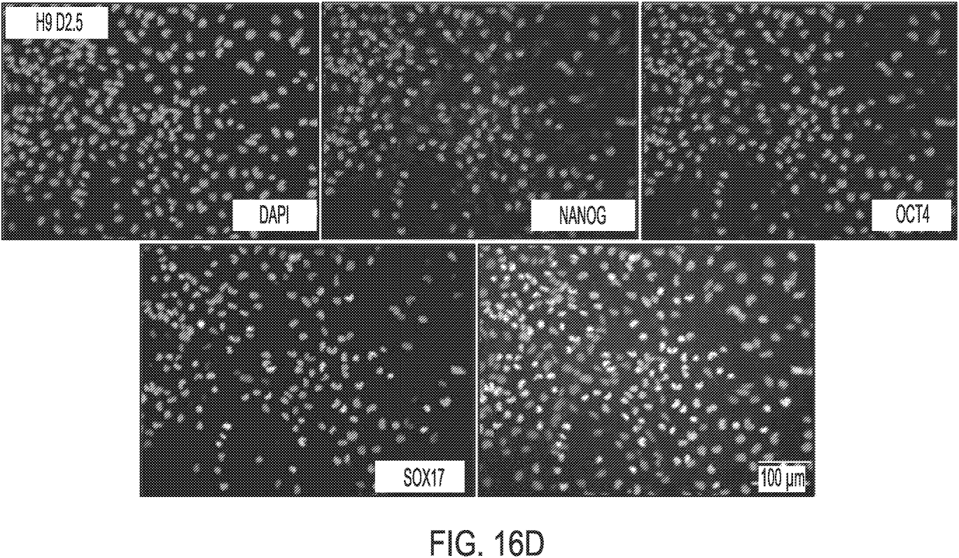

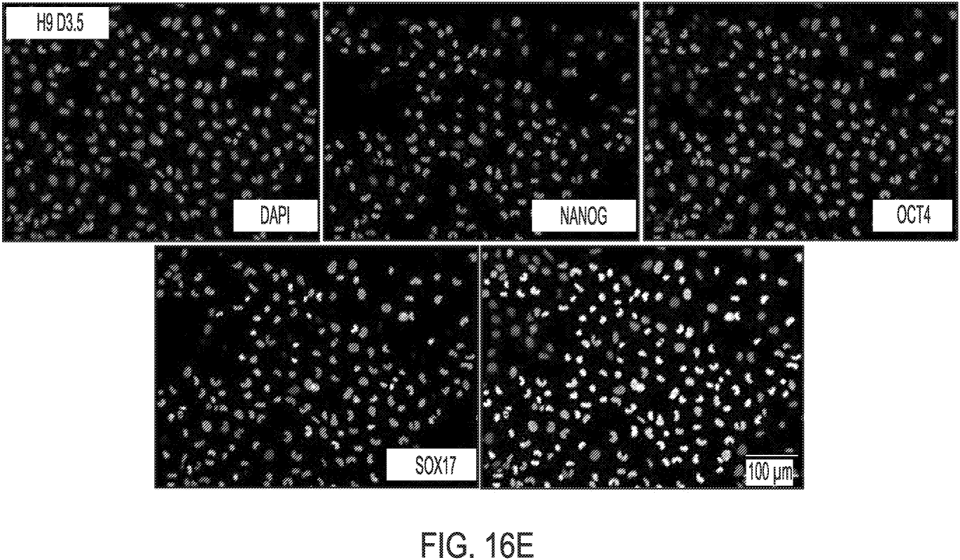

Integrated scRNAseq analysis of all populations revealed the stepwise changes in gene expression as pluripotent cells segue into D0.5 posterior epiblast and finally, D3.5 PGCs (FIG. 4E, FIG. 8D). While posterior epiblast markers BRACHYURY, MIXLI and NODAL were transiently expressed at D0.5, they were subsequently downregulated in D3.5 PGCs (FIG. 4E). This is consistent with the observed “repression of somatic genes” in fully-formed PGCs (Surani et al., 2007), although we note that these genes are nonetheless briefly expressed in their precursors (the posterior epiblast). Our side-by-side comparison of hPSC-derived PGCs and endoderm confirmed that they shared common markers SOX17 and PRDM1/BLIMP1 (Irie et al., 2014; Sasaki et al., 2015); however D3.5 PGCs expressed multiple unique markers that were not found in endoderm, including NANOG, NANOS3, TFAP2C, KLF4 and TCL1B (FIG. 4E, FIG. 9A). This thus discloses a single-cell transcriptional signature for hPSC-derived PGCs.

Finally, we investigated expression of pluripotency markers during germline differentiation; a quintessential feature of early germ cells (unlike most somatic cell-types) is that they express pluripotency transcription factors (reviewed by Magnusdottir and Surani, 2013; Surani et al., 2007). The prevailing model is that upon early differentiation, pluripotent cells initially downregulate pluripotency factors, but subsequently only cells allocated to the germline then “re-express” pluripotency factors (reviewed by Magnusdottir and Surani, 2013; Surani et al., 2007) (FIG. 4F, panel i). However, after computationally ordering differentiating cells in our scRNAseq dataset along an inferred “pseudotime”, OCT4 and NANOG were continuously expressed during the transition from pluripotency to posterior epiblast to PGCs (FIG. 4F, panel ii). This thus implies continuous expression of pluripotency factors in the transition from pluripotency to germline fate. We sought to experimentally validate this prediction by tracking NANOG expression at the single-cell level. To this end, we engineered NANOG-2A-YFP reporter hESCs wherein Cas9/AAV6 genome editing (Martin et al., 2019) was used to insert a 2A-YFP reporter immediately downstream of the NANOG gene without disrupting its coding sequence.

We found that NANOG was continuously expressed during the hPSC-to-germline transition, without evidence for NANOG downregulation followed by re-expression. Undifferentiated hPSCs, D0.5 posterior epiblast cells and D1.5 cells were largely NANOG⁺ CXCR4⁻, but then by D2.5-D3.5, a subpopulation continued to express NANOG but gained CXCR4, thus transitioning to NANOG⁺ CXCR4⁺ PGCs (FIG. 4F, panel iii; FIG. 9B). By contrast, by D2.5-D3.5, other cells lost NANOG, thus differentiating into NANOG⁻ CXCR4⁻ non-PGCs (FIG. 4F, panel iii; FIG. 9B). We independently confirmed these results obtained using the NANOG-2A-YFP reporter hESCs, by instead using intracellular flow cytometry to stain for NANOG protein itself (FIGS. 8B, 8C).

Therefore, after posterior epiblast formation, differentiating cells that “inherit” pluripotency factor expression from the posterior epiblast may progress forth to the germline, by contrast to the prevailing model that differentiating cells first downregulate and later “re-express” pluripotency genes (reviewed by Magnusdottir and Surani, 2013; Surani et al., 2007). Continued NANOG expression may thus serve as a bridge to link the pluripotent and PGC states.

### Discussion

The origins of human PGCs—as well as the exact lineage intermediate(s) and inductive extracellular signal(s) leading to their specification—remain incompletely understood. This has complicated efforts to efficiently generate PGCs in vitro from hPSCs. While germline and somatic cells are extremely different cell-types, the development of PGCs and primitive streak (PS) cells unexpectedly relies on certain shared signaling pathways and transcription factors (Aramaki et al., 2013; Hayashi et al., 2011; Kobayashi et al., 2017; Ohinata et al., 2009), and thus the relationship between these lineages has remained mysterious. We find that primed human PSCs (corresponding to post-implantation epiblast) are capable of generating PGCs (Kobayashi et al., 2017; Sasaki et al., 2015), but they must first be briefly exposed to PS-inducing signals (WNT and TGFβ to generate posterior epiblast, the common precursor to both PS and PGCs (Kobayashi et al., 2017). While sustained exposure to PS-inducing signals (e.g., WNT activation) further differentiates posterior epiblast into PS (Kobayashi et al., 2017; Loh et al., 2014; Loh et al., 2016), here we show that WNT suppression and BMP activation launches posterior epiblast into the PGC lineage and away from the PS lineage trajectory. In this model, the PGC and PS lineages are related-yet distinct—cell-types that arise from posterior epiblast intermediates and are segregated by mutually-exclusive signals (e.g., WNT).

Hence, there is both a temporal and combinatorial logic with which cells interpret WNT during germline specification. While WNT must be initially activated to differentiate hPSCs towards posterior epiblast, 12 hours later it inhibits PGC formation. At the 12-hour time point, we find that posterior epiblast cells have a unique molecular signature, denoted by pluripotency and PS marker co-expression. This is consistent with how E6.0-E6.25 mouse posterior epiblast expresses both Nanog and Brachyury immediately prior to overt formation of the primitive streak (Hoffman et al., 2013; Rivera-Perez and Magnuson, 2005). Posterior epiblast cells that receive both BMP and WNT signals respond by differentiating into PS (Loh et al., 2014; Loh et al., 2016), whereas those that receive BMP (in the absence of WNT) instead differentiate into PGCs. The molecular mechanisms underlying temporally dynamic reinterpretation of the WNT signal are enigmatic, but in mouse, a brief pulse of WNT can rapidly induce Brachyury, which subsequently ignites the expression of hallmark PGC markers (Aramaki et al., 2013). During this stepwise process, we find that pluripotency factors (OCT4 and NANOG) are continuously expressed in the transitions from hPSCs to posterior epiblast to PGCs. Thus, the continued expression of these pluripotency factors may serve as a molecular bridge between the pluripotent and germline states; cells that lose such expression may instead differentiate into somatic cells. This contrasts with an earlier hypothesis that differentiating cells lose pluripotency factor expression, but cells allocated to the germline “re-express” such factors (reviewed by Magnusdottir and Surani, 2013; Surani et al., 2007).

Finally, while we report a more efficient, reproducible and simplified monolayer system to generate human PGCs, despite our improvements, typically only one-third of cells (36.7%, across all hPSC lines) in the D3.5 population are PGCs (FIG. 2). Our single-cell RNA-sequencing survey revealed that the remaining “mis-differentiated” cells are mesoderm-like cells. Recent reports suggest that repression of Otx2 (in mouse), or overexpression of SOX17 and BLIMP1(in human) (Irie et al., 2014; Kobayashi et al., 2017), suffices to generate PGCs in vitro even in the absence of any exogenous signals. Delineating the upstream extracellular signals that repress OTX2, or that induce SOX17 and BLIMPl, is therefore paramount to further enhance the efficiency of human PGC formation in vitro.

### Materials and Methods

Human pluripotent stem cell (hPSC) culture. H1 hESCs (Thomson et al., 1998), H9 hESCs (Thomson et al., 1998), NANOS3-mCherry WIS1 hESCs (Irie et al., 2014), and SOX17-GFP H9 hESCs (Wang et al., 2011), BJC1 hiPSCs (Durruthy-Durruthy et al., 2014), BJC3 hiPSCs (Durruthy-Durruthy et al., 2014), and BIRc3 hiPSCs were routinely propagated feeder-free in mTeSR1 medium + 1% penicillin/streptomycin (StemCell Technologies) on cell culture plastics coated with Matrigel (Coming). Undifferentiated hPSCs were maintained at high quality with particular care to avoid any spontaneous differentiation, which would confound downstream differentiation.

In the NANOS3-mCherry hESC line, a 2A-mCherry fluorescent reporter was inserted immediately downstream of the NANOS3 gene without disrupting its coding sequence (Irie et al., 2014). In the SOX17-GFP hESC line, a GFP fluorescent reporter was inserted immediately after the SOX17 start codon, thus functionally invalidating one SOX17 allele (Wang et al., 2011). In the NANOG-2A-YFP hESC line, Cas9 RNP/AAV6-based genome editing (Martin et al., 2019) was used to insert a 2A-iCaspase9-2A-YFP fluorescent reporter immediately downstream of the NANOG gene without disrupting the NANOG coding sequence.

hPSC differentiation into PGCs. Undifferentiated hPSCs were maintained in mTeSR1 + 1% penicillin/streptomycin and enzymatically passaged (Accutase, 1:8-1:12 split) for differentiation. After overnight recovery in mTeSR1 + Thiazovivin (ROCK inhibitor, 5 µM), the following morning, hPSCs were briefly washed (DMEM/F12) and differentiated into posterior epiblast for 12 hours (100 ng/mL Activin + 3 µM CHIR99021 + 10 µM Y-27632) in aRB27 basal media, which comprised Advanced RPMI 1640 medium supplemented with 1% B27 supplement, 0.1 mM non-essential amino acids (NEAA), 100 U/ml Penicillin + 0.1 mg/ml Streptomycin, and 2 mM L-Glutamine (Kobayashi et al., 2017). Subsequently, cells were washed once more and treated with 40 ng/mL BMP4, 1 µM XAV939, and 10 µM Y-27632 for 24 hours, then 100 ng/mL SCF, 50 ng/mL EGF, 1 µM XAV939, and 10 µM Y-27632 for an additional 24 hrs, and finally 40 ng/mL BMP4, 100 ng/mL SCF, 50 ng/mL EGF, 1 µM XAV939, and 10 µM Y-27632 for 24 hours (all in aRB27 basal media). For comparison, published PGC differentiation protocols (Sasaki et al., 2015; Kobayashi et al., 2017) were performed as described previously.

Table 1 shows activators and inhibitors of signaling pathways used for differentiation.

| Item name | Company | Catalog no. |
|---|---|---|
| Activin A | Peprotech | 120-14E |
| BMP4 | R&D Systems | 314-BP-050 |
| CHIR99021 | Tocris | 4423 |
| EGF | Peprotech | AF-100-15 |
| LIF | Peprotech | 300-05 |
| SCF | Peprotech | 300-07 |
| TC-S 7001 | Tocris | 4961 |
| Thiazovivin | Tocris | 3845 |
| XAV939 | Tocris | 3748 |

Immunostaining Cells were grown on Matrigel-coated glass coverslips (Fisher). Cells were washed with PBS and then fixed with 4% PFA (paraformaldehyde) for 15 min. Coverslips were then washed with PBS, permeabilized with 0.2% Triton X-100 and blocked with PBS-BT (3% BSA + 0.1% Triton X-100 + 0.02% sodium azide in PBS) for at least 30 min. Coverslips were incubated with primary antibodies diluted in PBS-BT overnight, and then washed with PBS-BT, subsequently incubated with secondary antibodies and DAPI diluted in PBS-BT for 45 min, and then washed again. Finally, samples were mounted in ProLong Gold (Thermo Fisher Scientific) anti-fade solution. Images were acquired on a Zeiss LSM 500 scanning confocal microscope (Carl Zeiss, Jena, Germany) with a 40X objective.

Table 2 shows antibodies used for immunostaining.

| Antibody | Company | Cat. No. | Dilution |
|---|---|---|---|
| Anti-Blimp1 | eBioscience | 14-5963-82 | 1:50 |
| Anti-Hand1 | R&D Systems | AF3168 | 1:100 |
| Anti-Nanog | Abcam | ab109250 | 1:250 |
| Anti-Oct4 | BD | 611203 | 1:100 |
| Anti-Sox17 | R&D Systems | AF1924 | 1:500 |

High-throughput cell-surface marker screening. hESCs or differentiated PGCs were dissociated (using Accutase) and plated into individual wells of four 96-well plates, each well containing a distinct antibody against a human cell surface antigen, altogether totaling 371 unique cell-surface markers across multiple 96-well plates (LEGENDScreen PE-Conjugated Human Antibody Plates; Biolegend, 700007). For each LEGENDScreen experiment, approximately 10-70 million cells of each lineage were used. High-throughput cell-surface marker staining was largely done as per the manufacturer’s recommendations, and cells were stained with a viability dye (DAPI, 1.1 µM; Biolegend) prior to analysis on a CytoFLEX Flow Cytometer (Stanford Stem Cell Institute FACS Core). Stained cells were not fixed prior to FACS analysis. Sometimes, after lysophilized antibodies were reconstituted in LEGENDScreen plates they were aliquoted into a separate plate to generate replicates of antibody arrays. Undifferentiated H9 hESCs and SOX17-GFP H9 hESCs were used for LEGENDScreen analyses.

Flow cytometry and fluorescence-activated cell sorting (FACS) for cell-surface marker expression. hPSCs or their differentiated derivatives were dissociated using TrypLE Express (Gibco), were washed off the plate with FACS buffer (PBS + 0.1% BSA fraction V [Gibco] + 1 mM EDTA [Gibco] + 1% penicillin/streptomycin [Gibco]) and were pelleted by centrifugation (5 mins, 4° C.). Subsequently, cell pellets were directly resuspended in FACS buffer containing pre-diluted primary antibodies (listed below), thoroughly triturated to ensure a single cell suspension, and primary antibody staining was conducted for 30 mins on ice. Afterwards, cells were washed with an excess of FACS buffer and pelleted again, and this was conducted one more time. Finally, washed cell pellets were resuspended in FACS buffer containing 1.1 µM DAPI (Biolegend), and were strained through a 35 µm filter. Flow cytometry and sorting was conducted on a BD FACSAria II (Stanford Stem Cell Institute FACS Core).

Table 3 shows antibodies used for flow cytometry.

| Antibody | Company | Catalog Number | Dilution |
|---|---|---|---|
| CXCR4 APC | BD Biosciences | 560936 | 1:5 |
| GARP FITC | eBioscience | 11-9882-41 | 1:20 |
| PDGFRα PE | BD Biosciences | 556002 | 1:20 |

RNA extraction and reverse transcription. In general, RNA was extracted from undifferentiated or differentiated hPSC populations plated in 12-well format by lysing them with 350 µL RLT Plus Buffer per well. RNA was extracted with the RNeasy Plus Mini Kit (Qiagen) as per the manufacturer’s instructions. Generally 50-200 ng of total RNA was reverse-transcribed with the High Capacity cDNA Reverse Transcription Kit (Applied Biosystems) to generate cDNA libraries for qPCR.

Quantitative PCR (qPCR). Total cDNA was diluted 1:10-1:30 in H₂O and qPCR was performed with the SensiFAST SYBR Hi-ROX Kit (Bioline) with 10 µL qPCR reactions per well in a 384-well plate: each individual reaction contained 5 µL 2x SensiFAST SYBR qPCR Master Mix + 4.2 µL cDNA (totaling ~120 ng of cDNA) + 0.8 µL of 10 µM primer stock (5 µM forward + 5 µM reverse primers). In general, gene-specific primer pairs for qPCR were tested for (1) specificity of amplicon amplification (only one peak on a dissociation curve) and (2) linearity of amplicon amplification (linear detection of gene expression in cDNA samples serially diluted seven times over two orders of magnitude, with 90-110% efficiency of amplification deemed acceptably linear). After qPCR plates were prepared by arraying sample-specific cDNAs and gene-specific primers (listed below), they were sealed and briefly centrifuged (5 mins). 384-well qPCR plates and their adhesive sealing sheets were obtained from Thermo (AB1384 and AB0558, respectively). qPCR plates were run on a 7900HT Fast Real-Time PCR System (Applied Biosystems) with the following cycling parameters: initial dissociation (95° C., 2 mins) followed by 40 cycles of amplification and SYBR signal detection (95° C. dissociation, 5 seconds; 60° C. annealing, 10 seconds; followed by 72° C. extension, 30 seconds), with a final series of steps to generate a dissociation curve at the end of each qPCR run. During qPCR data analysis, the fluorescence threshold to determine Ct values was set at the linear phase of amplification.

Table 4 shows primers used for qPCR analysis.

| Gene Name | Forward | SEQ ID NO.: | Reverse | SEQ ID NO.: |
|---|---|---|---|---|
| BRACHYURY | TGCTTCCCTGAGACCCAGTT | 1 | GATCACTTCTTTCCTTTGCATCAAG | 2 |
| CDX2 | GGGCTCTCTGAGAGGCAGGT | 3 | CCTTTGCTCTGCGGTTCTG | 4 |
| KIT | GGATTCCCAGAGCCCACAA | 5 | ACATCCACTGGCAGTACAGAA | 6 |
| FOXA2 | GGGAGCGGTGAAGATGGA | 7 | TCATGTTGCTCACGGAGGAGTA | 8 |
| FZD8 | ATCGGCTACAACTACACCTACA | 9 | GTACATGCTGCACAGGAAGAA | 10 |
| GATA4 | TCCCTCTTCCCTCCTCAAAT | 11 | TCAGCGTGTAAAGGCATCTG | 12 |
| HHEX | CACCCGACGCCCTTTTACAT | 13 | GAAGGCTGGATGGATCGGC | 14 |
| MIXL1 | GGTACCCCGACATCCACTTG | 15 | TAATCTCCGGCCTAGCCAAA | 16 |
| NANOG | AGAACTCTCCAACATCCTGAACCTC | 17 | CTGAGGCCTTCTGCGTCACA | 18 |
| NANOS3 | ACTTACTGGCCAGGGCTACAC | 19 | ACTTCCCGGCACCTCTGAA | 20 |
| OCT4 (POU5F1) | AGTGAGAGGCAACCTGGAGA | 21 | ACACTCGGACCACATCCTTC | 22 |
| PAX5 | AAACCAAAGGTCGCCACAC | 23 | GTTGATGGAACTGACGCTAGG | 24 |
| PRDM1 | TCTCCAATCTGAAGGTCCACCTG | 25 | GATTGCTGGTGCTGCTAAATCTCTT | 26 |
| SHISA2 | TTCCTTTACTGAAGGGAGACGAAGG | 27 | CCATCCAAAGGAATCGTGCCATAAA | 28 |
| SOX17 | CGCACGGAATTTGAACAGTA | 29 | GGATCAGGGACCTGTCACAC | 30 |
| SOX2 | TGGACAGTTACGCGCACAT | 31 | CGAGTAGGACATGCTGTAGGT | 32 |
| TFAP2C | ATTAAGAGGATGCTGGGCTCTG | 33 | CACTGTACTGCACACTCACCTT | 34 |
| TPCP2L1 | AGCTCAAAGTTGTCCTACTGCC | 35 | TTCTAACCCAAGCACAGATCCC | 36 |
| VASA (DDX4) | GTGCCCTATGTGCCGTTAC | 37 | GGCTGACGTTGGACTGAGG | 38 |

Single cell RNA-sequencing. Differentiated cells of various stages were dissociated and washed twice in wash buffer (0.04% Bovine Serum Albumin in Ca2+/Mg2+-free PBS) and counted on the Countess II automated cell counter (Thermo Fisher). For each cell population, 10,400 cells were loaded per lane on the 10x Chromium platform (aiming to capture 6,000 cells). Cells were then processed for cDNA synthesis and library preparation using 10X Chromium Version 2 chemistry (Cat# 120234) as per the manufacturer’s protocol. cDNA and libraries were checked for quality on the Agilent 4200 Tape Station platform and their concentration was quantified by KAPA qPCR. Libraries were sequenced HiSeq 4000 (Illumina) to a depth of, at a minimum, 70,000 reads per cell.

Single cell RNA-seq computational analysis. Illumina base call files were converted to FASTQ files using the Cell Ranger v2.0 program. FASTQ files were then aligned to the human hg19 genome using Cell Ranger. The Seurat R package (v2.3.1) was used for subsequent analyses. Cells from all the various timepoints were first combined into a single Seurat object. For quality control, we first filtered out low-quality cells that expressed less than 2,500 genes; we also excluded cells that expressed more than 7,500 genes (which would imply doublets) or that expressed more than 0.15% mitochondrial genes (indicative of dead cells in this dataset) were filtered out. Counts were normalized and scaled by a factor of 10,000. To adjust for cell cycle effects, S phase and G2M genes were regressed out before Principal Component Analysis (PCA) was performed using the highly-variable genes.

For further analyses, 1,000 cells were randomly sampled from each of the 5 data sets (D0, D0.5, D3.5 sorted, D3.5 unsorted and D2 definitive endoderm) and then combined into a new file for further analysis. The top six principal components were used for clustering using the Shared Nearest Neighbor (SNN) algorithm, which was implemented via the FindCluster function in Seurat. Clusters were visualized using t-SNE plot with 3-dimensional embedding. Differentially expressed genes between clusters were identified using thee Wilcoxon rank sum test which was performed via the Seurat package. For all other independent library analyses, the following numbers of principal components (NPC) were used: Day 3.5 sorted library - npc=15; Day 3.5 unsorted library - npcs=10; Day 0 vs Day 0.5 analysis – npcs=10. For identifying enriched transcription factors (TFs), surface expressed proteins and ligands and receptors in each cell population, first a list of curated TF, surface proteins, ligands and receptor database were obtained from Bioinformatics. 2013 Oct 1;29(19):2519-20, PLoS One. 2015 Apr 20;10(3):e0121314 and Nat Genet. 2001 Nov;29(3):295-300. respectively. These lists were used to subset the differentially expressed genes between clusters to identify the enriched TFs, surface proteins and ligands and receptors. All plots were generated using Seurat and ggplot2 R packages. For trajectory analysis, only D0, D0.5 and D3.5 unsorted libraries were used. Only genes expressed in at least 10 cells were used. The top 1000 differentially expressed genes were used for ordering and dimensional reduction was done with DDRTree.

Table 5. Surface marker screen of PGCs, non-PGCs and undifferentiated hPSCs. The table shows percentages of surface markers expressed in undifferentiated hPSC (D0), D3.5 SOX17-GFP⁺ PGCs and D3.5 SOX17-GFP⁻ non-PGCs identified from LEGENDScreen. To discriminate PGCs vs. non-PGCs, SOX17-GFP hESCs were differentiated for D3.5 and then subgated on GFP⁺ and GFP⁻ before further analysis of surface marker expression. %PE+ indicates the percentage of cells bound to the PE-conjugated antibody, demonstrating detection of the indicated marker.

| Well ID | Antigen | % PE+ in PGCs | % PE+ in non-PGCs | % PE+ in H1 UD |
|---|---|---|---|---|
| A1 | Blank | 0 | 0 | 0.13 |
| A2 | IgG Isotype Ctrl | 0 | 5.43548E-05 | 0.058 |
| A3 | CCR10 | 0.004014824 | 0.002736394 | 0.37 |
| A4 | CD278 | 0 | 5.9713E-05 | 0.038 |
| A5 | IFN-y R b chain | 0 | 0.000536693 | 0.39 |
| A6 | IgG1, κ Isotype Ctrl | 0 | 0.000273205 | 0.21 |
| A7 | CD46 | 0.956006768 | 0.948720542 | 99.9 |
| A8 | CD70 | 0 | 0.000506329 | 0.25 |
| A9 | CD1a | 0.001604564 | 0.000141537 | 0.78 |
| A10 | CD2 | 0 | 0 | 0.039 |
| A11 | β2-microglobulin | 0.989541432 | 0.995443038 | 99.7 |
| A12 | B7-H4 | 0.035621199 | 0.065344932 | 0.61 |
| B1 | Cadherin 11 | 0 | 0.000222848 | 0.23 |
| B2 | CD10 | 0.542635659 | 0.426545086 | 99.7 |
| B3 | CD100 | 0.003861951 | 0.001114375 | 99.3 |
| B4 | CD103 | 0 | 0 | 0.1 |
| B5 | CD105 (Endoglin) | 0.092436975 | 0.210526316 | 77 |
| B6 | CD106 | 0.011437908 | 0.000384857 | 0.19 |
| B7 | CD107a | 0.705882353 | 0.509109312 | 99.7 |
| B8 | CD107b | 0.040365575 | 0.071370641 | 18.6 |
| B9 | CD109 | 0.11965812 | 0.00161845 | 44.2 |
| B10 | CD111 | 0.64 | 0.51266464 | 99.7 |
| B11 | CD112 | 0.846774194 | 0.821681864 | 99.9 |
| B12 | CD114 | 0 | 0.000192661 | 0.65 |
| C1 | CD116 | 0.002923757 | 0.032323437 | 0.9 |
| C2 | CD117 | 0.12195122 | 0.083729877 | 99.6 |
| C3 | CD119 | 0.328244275 | 0.432624113 | 99.8 |
| C4 | CD11a | 0 | 5.66331E-05 | 0.3 |
| C5 | CD11b | 0 | 7.33482E-05 | 0.13 |
| C6 | CD122 | 0.00137503 | 1.81354E-05 | 0.087 |
| C7 | CD123 | 0.004303976 | 0.001620089 | 0.31 |
| C8 | CD126 | 0.098484848 | 0.000232975 | 0.26 |
| C9 | CD127 | 0 | 3.48872E-05 | 0.044 |
| C10 | CD13 | 0.610687023 | 0.792299899 | 54.6 |
| C11 | CD131 | 0 | 0.000141824 | 0.16 |
| C12 | CD134 | 0 | 0.000111436 | 0.35 |
| D1 | CD135 | 0 | 0.000142112 | 0.26 |
| D2 | CD137 | 0 | 3.39745E-05 | 0.13 |
| D3 | 4-1BB Ligand | 0.025245442 | 0.047377498 | 1.23 |
| D4 | CD138 | 0.119402985 | 0.117527862 | 0.49 |
| D5 | CD14 | 0.007795889 | 0.007711039 | 0.29 |
| D6 | CD140a | 0.057437408 | 0.359675785 | 0.12 |
| D7 | CD140b | 0 | 0.006790311 | 0.53 |
| D8 | CD141 | 0.062927497 | 0.06830407 | 18.9 |
| D9 | CD142 | 0.22962963 | 0.174442191 | 99.6 |
| D10 | CD143 | 0.242857143 | 0.097919838 | 25 |
| D11 | CD146 | 0.928838951 | 0.647416413 | 91.7 |
| D12 | CD148 | 0.529411765 | 0.276315789 | 99.7 |
| E1 | CD15 | 0.008649368 | 0.004060914 | 2.04 |
| E2 | CD150 | 0 | 0 | 0.022 |
| E3 | CD151 | 0.734939759 | 0.981588852 | 99.3 |
| E4 | CD154 | 0 | 0.000162081 | 0.16 |
| E5 | CD156c | 0.983836964 | 0.923389143 | 99.8 |
| E6 | CD158e1 | 0 | 2.31776E-05 | 0.19 |
| E7 | CD16 | 0 | 5.17215E-05 | 0.21 |
| E8 | CD161 | 0 | 4.80707E-05 | 0.068 |
| E9 | CD162 | 0 | 9.06706E-05 | 0.67 |
| E10 | CD163 | 0.001346333 | 0.000131828 | 0.2 |
| E11 | CD164 | 0.372262774 | 0.585192698 | 99.3 |
| E12 | CD165 | 0.951374207 | 0.937119675 | 99.8 |
| F1 | CD166 | 0.809859155 | 0.740101523 | 99.8 |
| F2 | CD169 | 0 | 0.000232975 | 0.21 |
| F3 | CD170 | 0.001370382 | 0.000587889 | 7.92 |
| F4 | CD172a/b (SIRPα/β) | 0.02739726 | 0.084542779 | 82.4 |
| F5 | CD172g (SIRPγ) | 0.002096945 | 0.000172559 | 0.16 |
| F6 | CD178 | 0 | 0.00016241 | 0.32 |
| F7 | CD179a | 0.001065977 | 0.001420743 | 0.31 |
| F8 | CD179b | 0 | 0.000111663 | 0.1 |
| F9 | CD18 | 0 | 0.000131962 | 0.069 |
| F10 | CD180 | 0 | 1.58213E-05 | 0.13 |
| F11 | CD182 | 0 | 3.4009E-05 | 1.31 |
| F12 | CD183 | 0.014084507 | 0.018772197 | 1.59 |
| G1 | CD185 | 0 | 0.00026389 | 0.17 |
| G2 | CD19 | 0 | 2.76135E-05 | 0.062 |
| G3 | CD191 | 0.007490637 | 0.00203252 | 0.5 |
| G4 | CD194 | 0.006233625 | 0.004667208 | 0.46 |
| G5 | CD1b | 0 | 0.000578933 | 0.31 |
| G6 | CD1c | 0 | 5.8313E-05 | 1.19 |
| G7 | CD200 | 0.968882603 | 0.748478702 | 99.9 |
| G8 | CD200R | 0 | 0.000283895 | 0.24 |
| G9 | CD202b | 0.018361582 | 0.363083164 | 50.5 |
| G10 | CD203c | 0 | 0.000354844 | 0.45 |
| G11 | CD205 | 0.001084537 | 0.000172384 | 50 |
| G12 | CD206 | 0 | 0.000364978 | 0.25 |
| H1 | CD207 | 0 | 0.000253485 | 0.042 |
| H2 | CD21 | 0 | 8.53882E-05 | 73.1 |
| H3 | CD213α1 | 0 | 0.000830952 | 0.7 |
| H4 | CD213α2 | 0 | 0.002329586 | 0.24 |
| H5 | CD218a | 0 | 0.00152207 | 0.12 |
| H6 | CD221 | 0.696296296 | 0.713272543 | 99.5 |
| H7 | CD223 (LAG-3) | 0 | 0.000152261 | 0.19 |
| H8 | CD226 | 0 | 0.000567629 | 0.24 |
| H9 | CD227 | 0.076212471 | 0.161094225 | 90.9 |
| H10 | CD229 | 0.000948006 | 9.25878E-05 | 0.19 |
| H11 | CD23 | 0 | 1.25759E-05 | 0.043 |
| H12 | CD231 | 0 | 0.000314622 | 0.71 |
| A1 | Blank | 0 | 0 | 0.041 |
| A2 | CD244 (2B4) | 0 | 0.000151645 | 0.53 |
| A3 | CD245 | 0.038461538 | 0.165656566 | 98.7 |
| A4 | CD25 | 0.025210084 | 0.016293897 | 0.12 |
| A5 | CD252 | 0 | 0.00059681 | 1.23 |
| A6 | CD261 | 0 | 0.00024261 | 96.4 |
| A7 | CD262 | 0.235294118 | 0.320525784 | 99.9 |
| A8 | CD263 | 0.028571429 | 0.153690597 | 1.91 |
| A9 | CD266 | 0.017175573 | 0.032649348 | 99.2 |
| A10 | CD268 | 0.005039891 | 0.005966225 | 11.6 |
| A11 | CD27 | 0.007806504 | 0.071154235 | 0.14 |
| A12 | CD271 | 0.583333333 | 0.342770475 | 99.7 |
| B1 | CD275 | 0.418181818 | 0.177957533 | 99 |
| B2 | CD276 | 0.994600739 | 0.999412706 | 99.8 |
| B3 | CD277 | 0.003142475 | 0.000838528 | 0.89 |
| B4 | CD279 | 0 | 0.000625871 | 1.88 |
| B5 | CD28 | 0 | 3.50493E-05 | 0.33 |
| B6 | CD29 | 0.991177432 | 0.998789346 | 99.8 |
| B7 | CD290 | 0 | 0.000242856 | 0.22 |
| B8 | CD298 | 1 | 0.999311518 | 98.7 |
| B9 | CD3 | 0 | 0.000232739 | 0.75 |
| B10 | CD30 | 0 | 0.000141537 | 75.2 |
| B11 | CD300c | 0.003650179 | 0.000910102 | 0.6 |
| B12 | CD309 | 0.008068302 | 0.15065723 | 99.4 |
| C1 | CD31 | 0.02027027 | 0.000273205 | 0.36 |
| C2 | CD314 | 0 | 6.62241E-05 | 0.36 |
| C3 | CD317 | 0.756756757 | 0.895748988 | 99.4 |
| C4 | CD324 | 0.743801653 | 0.55465587 | 99.5 |
| C5 | CD325 | 0 | 0.0006986 | 0.61 |
| C6 | CD328 | 0 | 0.000162081 | 0.13 |
| C7 | CD33 | 0 | 0.000232739 | 0.43 |
| C8 | CD334 | 0 | 0 | 16.9 |
| C9 | CD335 | 0 | 2.59103E-05 | 0.043 |
| C10 | CD336 | 0 | 3.21852E-05 | 0.24 |
| C11 | CD337 | 0 | 2.42909E-05 | 0.27 |
| C12 | CD34 | 0 | 0.000505817 | 4.12 |
| D1 | CD340 | 0.746268657 | 0.953877344 | 99.8 |
| D2 | CD344 | 0.575 | 0.395748988 | 96.5 |
| D3 | CD35 | 0 | 0.000748427 | 3.22 |
| D4 | CD354 | 0.330769231 | 0.434650456 | 0.24 |
| D5 | CD360 | 0 | 0.00161845 | 8.46 |
| D6 | CD365 | 0.352941176 | 0.509109312 | 0.28 |
| D7 | CD366 | 0 | 0.000323454 | 0.26 |
| D8 | CLEC4A | #DIV/0! | #DIV/0! | 0.22 |
| D9 | CD36L1 | 0 | 0 | 99.5 |
| D10 | CD38 | 0 | 0.000101204 | 2.15 |
| D11 | CD39 | 0.228070175 | 0.035229804 | 47.2 |
| D12 | CD4 | 0.156521739 | 0.004048583 | 11.1 |
| E1 | CD40 | #DIV/0! | #DIV/0! | 13.2 |
| E2 | CD41 | #DIV/0! | #DIV/0! | 0.064 |
| E3 | CD42b | 0.001707822 | 0.00293552 | 0.066 |
| E4 | CD43 | 0 | 2.35824E-05 | 0.55 |
| E5 | CD44 | 0 | 0 | 79.7 |
| E6 | CD45 | 0.007104897 | 0.018634798 | 0.2 |
| E7 | CD47 | 0.097560976 | 0.270242915 | 99.6 |
| E8 | CD48 | 0 | 0.000151799 | 2.17 |
| E9 | CD49a | 0.933823529 | 0.855983773 | 99.7 |
| E10 | CD49b | 0 | 0.000425351 | 99.8 |
| E11 | CD49c | 0.785714286 | 0.899433428 | 99.8 |
| E12 | CD49d | 0.625 | 0.170040486 | 62.1 |
| F1 | CD5 | #DIV/0! | #DIV/0! | 0.2 |
| F2 | CD50 | #DIV/0! | #DIV/0! | 51.8 |
| F3 | CD54 | 0 | 3.36021E-05 | 99.7 |
| F4 | CD55 | 0.055319149 | 0.007285974 | 99.8 |
| F5 | CD56 (NCAM) | 0.172727273 | 0.019012945 | 99.8 |
| F6 | CD58 | 0.933758978 | 0.841945289 | 99.9 |
| F7 | CD6 | 0.611940299 | 0.924293098 | 0.26 |
| F8 | CD61 | 0.971867008 | 0.927031677 | 0.043 |
| F9 | CD62E | 0 | 0.00015313 | 0.13 |
| F10 | CD62L | 0 | 6.28503E-05 | 0 |
| F11 | CD62P | 0 | 4.74674E-05 | 0.11 |
| F12 | CD63 | 0.007307551 | 0.003238211 | 99.5 |
| G1 | CD64 | #DIV/0! | #DIV/0! | 0.44 |
| G2 | CD69 | #DIV/0! | #DIV/0! | 0.13 |
| G3 | CD73 | 0.00148616 | 0.000151953 | 20.8 |
| G4 | CD74 | 0.001645725 | 0.004657756 | 0.39 |
| G5 | CD79b | 0.003784076 | 0.001215559 | 0.76 |
| C6 | CD8a | 0 | 0.000455719 | 0.22 |
| G7 | CD80 | 0 | 0.000506842 | 0.064 |
| G8 | CD81 | #DIV/0! | #DIV/0! | 99.5 |
| G9 | CD82 | 0 | 0.000161917 | 87.5 |
| G10 | CD83 | 0.99413694 | 0.945001519 | 3.72 |
| G11 | CD85 | 0.010629599 | 0.296558704 | 0.067 |
| G12 | CD85k | 0.002867722 | 0.002430626 | 0.088 |
| H1 | CD87 | #DIV/0! | #DIV/0! | 0.37 |
| H2 | CD89 | #DIV/0! | #DIV/0! | 0.13 |
| H3 | CD8a | 0.002513352 | 0.000243102 | 2.47 |
| H4 | CD9 | 0 | 0.000162081 | 99.7 |
| H5 | CD90 | 0 | 0.000131695 | 99.9 |
| H6 | CD93 | 0.971193416 | 0.905041506 | 1.41 |
| H7 | CD94 | 0.99662338 | 0.999483026 | 0.13 |
| H8 | CD95 | #DIV/0! | #DIV/0! | 78.2 |
| H9 | CD96 | 0 | 0.000253229 | 0.69 |
| H10 | CD97 | 0 | 0.000253485 | 9.77 |
| H11 | CD99 | 0 | 0.000111324 | 81.7 |
| H12 | CXCL16 | 0 | 0.000982806 | 0.29 |
| A1 | Blank | 0 | 0 | 0.022 |
| A2 | DLL1 | 0.027072758 | 0.020957781 | 0.68 |
| A3 | DLL4 | 0 | 0 | 0.1 |
| A4 | DR3 | 0 | 0 | 0.2 |
| A5 | EGFR | 0 | 0.00014168 | 64.5 |
| A6 | CD357 | 0 | 4.76698E-05 | 0.69 |
| A7 | GPR19 | 0 | 0.000656798 | 68.9 |
| A8 | GPR56 | 0 | 0.00090091 | 0.18 |
| A9 | HLA-E | 0 | 0.000485103 | 0.43 |
| A10 | HVEM | 0 | 0.001214329 | 0.34 |
| A11 | Ig light chain κ | 0 | 0 | 20.1 |
| A12 | IgM | 0 | 0 | 0.1 |
| B1 | CD360 | 0 | 0.000141824 | 4.55 |
| B2 | Integrin α9β1 | 0.760330579 | 0.747975709 | 91.3 |
| B3 | Jagged 2 | 0 | 0.000565908 | 0.47 |
| B4 | Ksp37 | 0 | 9.39183E-05 | 0.06 |
| B5 | LAP | 0 | 0.002627324 | 0.097 |
| B6 | LY6G6D | 0 | 0 | 0.11 |
| B7 | MERTK | 0.711864407 | 0.504048583 | 99.8 |
| B8 | MSC | 0.718181818 | 0.602628918 | 99.8 |
| B9 | MSC,N PC | 0.382113821 | 0.36437247 | 9.21 |
| B10 | TNAP | 0.95026643 | 0.68958544 | 99.7 |
| B11 | MUC-13 | 0 | 0.000121566 | 0.27 |
| B12 | NKp80 | 0 | 0.000121443 | 0.23 |
| C1 | Notch 1 | 0.00341946 | 0.000454798 | 97.2 |
| C2 | Notch3 | 0 | 4.44762E-05 | 44.6 |
| C3 | Notch 4 | 0 | 6.49755E-05 | 0.51 |
| C4 | NPC | 0.243243243 | 0.264914055 | 8.34 |
| C5 | CD352 | 0 | 0.000161917 | 0.35 |
| C6 | PSMA | 0 | 7.55004E-05 | 0.091 |
| C7 | ROR1 | 0.831932773 | 0.817813765 | 99.8 |
| C8 | Siglec-10 | 0 | 0.019746835 | 0.35 |
| C9 | CD328 | 0 | 0.000121443 | 0.17 |
| C10 | Siglec-8 | 0 | 4.64084E-05 | 0.16 |
| C11 | Siglec-9 | 0 | 0.000121566 | 0.15 |
| C12 | SSEA-5 | 0.996748701 | 0.919679935 | 51.7 |
| D1 | SUSD2 | 0.75 | 0.662285137 | 96.3 |
| D2 | TCR α/β | 0 | 0.000131562 | 0.53 |
| D3 | TCR y/δ | 0 | 0.000363872 | 0.53 |
| D4 | Tim-4 | 0 | 4.26095E-05 | 0.37 |
| D5 | TLT-2 | 0 | 7.88613E-05 | 0.22 |
| D6 | TM4SF20 | 0 | 0 | 0.12 |
| D7 | TRA-2-49 | 1 | 0.999120208 | 99.8 |
| D8 | TRA-2-54 | 1 | 0.99838155 | 99.9 |
| D9 | TSLPR | 0 | 8.6949E-05 | 0.34 |
| D10 | VEGFR-3 | 0 | 0.000171861 | 47.2 |
| D11 | IgG2a, κ Isotype Ctrl | 0 | 0.000181969 | 0.19 |
| D12 | APCDD1 | 0.035532995 | 0.005362744 | 0.82 |
| E1 | CD272 | 0 | 0.000222622 | 0.24 |
| E2 | CD198 | 0.002278379 | 0.002837454 | 0.84 |
| E3 | CCRL2 | 0 | 0.000393784 | 0.27 |
| E4 | CD102 | 0 | 0.000243102 | 3.9 |
| E5 | CD104 | 0 | 0.000151799 | 0.5 |
| E6 | CD124 | 0 | 3.5019E-05 | 11.3 |
| E7 | CD130 | 0.504273504 | 0.201417004 | 0.22 |
| E8 | CD144 | 0 | 0.000212505 | 0.55 |
| E9 | CD152 (CTLA-4) | 0 | 0.00014168 | 0.32 |
| E10 | CD155 | 0.994514385 | 0.817629179 | 99.9 |
| E11 | CD158b | 0 | 0.000161753 | 0.46 |
| E12 | CD184 | 0.79047619 | 0.017072432 | 1.21 |
| F1 | CD186 | 0 | 0.000151799 | 0.082 |
| F2 | CD192 | 0 | 0.000313667 | 0.16 |
| F3 | CD197 | 0.004314637 | 0.013044797 | 2.73 |
| F4 | CD199 | 0.016236867 | 0.007780135 | 1.29 |
| F5 | CD209 | 0 | 4.55445E-05 | 0.083 |
| F6 | CD217 | 0 | 0.002932551 | 85.3 |
| F7 | CD230 (Prion) | 0 | 0.008808343 | 11.5 |
| F8 | CD24 | 0.982800983 | 0.997367355 | 72.7 |
| F9 | CD243 | 0 | 0.000819166 | 5.25 |
| F10 | CD26 | 0.2 | 0.000313984 | 6.47 |
| F11 | CD269 | 0 | 3.38715E-05 | 0.12 |
| F12 | CD282 | 0 | 0.000253229 | 0.33 |
| G1 | CD284 | 0 | 0.000658795 | 0.9 |
| G2 | CD301 | 0 | 0.002632645 | 0.64 |
| G3 | CD303 | 0 | 0 | 0.033 |
| G4 | CD304 | 0 | 0.001416431 | 0.043 |
| G5 | CD307e | 0 | 0 | 0.18 |
| G6 | CD323 | 0.845528455 | 0.867408907 | 99.8 |
| G7 | CD357 | 0.002531391 | 0 | 0.68 |
| G8 | CD36 | 0 | 0.001215559 | 2.23 |
| G9 | CD369 | 0 | 0.000354126 | 0.21 |
| G10 | CD370 | 0 | 0.000192465 | 0.084 |
| G11 | CD371 | 0 | 0.002529084 | 0.12 |
| G12 | CD45RO | 0.002112196 | 5.13132E-05 | 0.031 |
| H1 | CD51 | 0.974325214 | 0.970854067 | 99.8 |
| H2 | CD59 | 0.969793323 | 0.999351991 | 100 |
| H3 | CD7 | 0.210526316 | 0.241658241 | 18.6 |
| H4 | CD71 | 0.875912409 | 0.734279919 | 99.7 |
| H5 | CD84 | 0 | 0.000121566 | 0.42 |
| H6 | CD88 | 0 | 0.00014168 | 0.19 |
| H7 | CD355 | 0 | 0.000121566 | 0.19 |
| H8 | erbB3 | 0.504065041 | 0.127659574 | 99.3 |
| H9 | FPR3 | 0 | 0.000192465 | 0.52 |
| H10 | Ganglioside GD2 | 0.045226131 | 0.177125506 | 5.41 |
| H11 | GPR83 | 0 | 0.001416431 | 0.28 |
| H12 | HLA-A,B,C | 0.336283186 | 0.760121457 | 99.9 |
| A1 | Blank | 0 | 0 | 0.02 |
| A2 | HLA-DR | 0 | 0.000303245 | 8.62 |
| A3 | Ig light chain λ | 0 | 0.000192076 | 0.1 |
| A4 | IgD | 0 | 0.000282463 | 0.027 |
| A5 | IL-28RA | 0 | 6.92571E-05 | 0.17 |
| A6 | integrin β5 | 0.688073394 | 0.701718908 | 99.6 |
| A7 | KLRG1 | 0 | 0.000272653 | 0.45 |
| A8 | LOX-1 | 0.004740309 | 0.000181785 | 99.6 |
| A9 | MICA/MICB | 0.011406844 | 0.002526529 | 99.3 |
| A10 | SUSD2 | 0.787037037 | 0.660262892 | 98.1 |
| A11 | Notch 2 | 0.006504424 | 0.062025701 | 88.5 |
| A12 | TACSTD2 | 0.108108108 | 0.217611336 | 17.9 |
| B1 | TIGIT (VSTM3) | 0 | 0.000111211 | 0.027 |
| B2 | IgG2b, κ Isotype Ctrl | 0 | 0.001818549 | 0.49 |
| B3 | C3aR | 0 | 0.000201979 | 0.22 |
| B4 | CCX-CKR (CCRL1) | 0 | 0.000445146 | 6.75 |
| B5 | CD11c | 0 | 0.003234937 | 0.061 |
| B6 | CD129 | 0 | 0.00057659 | 0.39 |
| B7 | CD158 | 0 | 0.000232504 | 4.24 |
| B8 | CD181 | 0 | 0.002627324 | 95.7 |
| B9 | CD193 | 0 | 0.002627324 | 0.27 |
| B10 | CD196 | 0.015228426 | 0.07446701 | 0.86 |
| B11 | CD1d | 0.155963303 | 0.196157735 | 24 |
| B12 | CD20 | 0 | 7.08452E-05 | 0.041 |
| C1 | CD22 | 0 | 6.83472E-05 | 0.094 |
| C2 | CD220 | 0.013282732 | 0.003335692 | 5.05 |
| C3 | CD235ab | 0 | 8.21975E-05 | 0.025 |
| C4 | CD258 | 0 | 0.000708 | 0.93 |
| C5 | CD274 | 0.00424318 | 0.016078471 | 0.89 |
| C6 | CD319 | 0 | 0.000615782 | 0.16 |
| C7 | CD32 | 0 | 0.000909183 | 0.94 |
| C8 | CD326 | 1 | 0.952717721 | 99.6 |
| C9 | CD338 | 0.025069638 | 0.115267947 | 4.54 |
| C10 | CD368 | 0 | 0.000323128 | 0.21 |
| C11 | CD45RA | 0 | 0.000252717 | 0.02 |
| C12 | CD45RB | 0 | 4.12104E-05 | 0.02 |
| D1 | CD49e | 1 | 0.999484072 | 99.6 |
| D2 | CD52 | 0 | 0.071414121 | 0.85 |
| D3 | CD66a/c/e | 0.010484652 | 0.012121212 | 1.32 |
| D4 | CD85h | 0 | 0.00031335 | 0.14 |
| D5 | CD85 | 0 | 0.000192076 | 0.27 |
| D6 | CD86 | 0 | 0.000545157 | 11.6 |
| D7 | CD92 | 0.853658537 | 0.866261398 | 95.5 |
| D8 | CXCR7 | 0.00705271 | 0.011422218 | 1.02 |
| D9 | Delta Opioid Receptor | 0.007075704 | 0.005966225 | 0.21 |
| D10 | Dopamine Receptor D1 (DRD1) | 0 | 0.00444714 | 0.081 |
| D11 | EphA2 | 0.517857143 | 0.468149646 | 99.5 |
| D12 | FcεRIα | 0 | 0.000263355 | 0.34 |
| E1 | GARP | 0.252173913 | 0.522750253 | 0.78 |
| E2 | CD215 | 0.006622517 | 0.006374583 | 0.25 |
| E3 | Lymphotoxin β Receptor | 0.006085383 | 0.001011122 | 21.3 |
| E4 | MRGX2 | 0 | 0.000828433 | 0.2 |
| E5 | TMEM8A | 0.275229358 | 0.258585859 | 99.4 |
| E6 | CD254 | 0.005076142 | 0.000151492 | 0.42 |
| E7 | CD318 | 0.576576577 | 0.175935288 | 99.7 |
| E8 | IgG3,k Isotype Ctrl | 0 | 0.000546813 | 0.48 |
| E9 | CD255 | 0.003225923 | 0.006776575 | 0.99 |
| E10 | SSEA-4 | 0.964083176 | 0.994642677 | 99.9 |
| E11 | IgM, κ Isotype Ctrl | 0 | 0.000192076 | 0.36 |
| E12 | Sialyl Lewis X (dimeric) | 0 | 0.000313033 | 0.1 |
| F1 | TRA-1-81 | 0.5 | 0.186234818 | 98.7 |
| F2 | CD160 | 0.00307235 | 0.005055612 | 1.32 |
| F3 | CD57 | 0.451327434 | 0.479757085 | 99.7 |
| F4 | CD66b | 0 | 0.000232504 | 0.27 |
| F5 | TRA-1-60-R | 0.491525424 | 0.324898785 | 99.5 |
| F6 | IgG1, κ Isotype Ctrl | 0 | 9.80692E-05 | 0.33 |
| F7 | CD115 | 0 | 0.001517451 | 0.66 |
| F8 | CD201 | 0.113043478 | 0.41194332 | 96.5 |
| F9 | IgG2a, κ Isotype Ctrl | 0 | 7.09464E-05 | 0.17 |
| F10 | CD120b | 0 | 0.000505306 | 37.4 |
| F11 | CD210 | 0 | 0.001416431 | 0.55 |
| F12 | CD267 | 0 | 0.000181785 | 0.1 |
| G1 | CD294 | 0.474576271 | 0.022046926 | 0.3 |
| G2 | CD49f | 0.940594059 | 0.388888889 | 99.6 |
| G3 | CD85 | 0 | 3.23191E-05 | 0.31 |
| G4 | CD85d | 0 | 0.000222848 | 0.37 |
| G5 | IgG Fc | 0 | 3.2456E-05 | 0.052 |
| G6 | Integrin β7 | 0 | 0 | 0.088 |
| G7 | XCR1 | 0 | 0.00059681 | 0.3 |
| G8 | Podoplanin | 0.99707998 | 0.872598584 | 98.7 |
| G9 | IgG2b, κ Isotype Ctrl | 0.00263269 | 8.89708E-05 | 0.32 |
| G10 | CD132 | 0.0078125 | 0.008004864 | 1.07 |
| G11 | CD195 | 0.01384083 | 0.028340081 | 0.041 |
| G12 | CX3CR1 | 0 | 0.000111211 | 0.2 |
| H1 | IgM, κ Isotype Ctrl | 0 | 0.000364978 | 0.047 |
| H2 | SSEA-3 | 0.006654536 | 0.007077856 | 68.4 |
| H3 | Blank | 0 | 0 | 0.28 |
| H4 | Blank | #DIV/0! | #DIV/0! | 0.14 |
| H5 | Blank | #DIV/0! | #DIV/0! | 0.094 |
| H6 | Blank | #DIV/0! | #DIV/0! | 0.029 |
| H7 | Blank | #DIV/0! | #DIV/0! | 0.028 |
| H8 | Blank | #DIV/0! | #DIV/0! | 0 |
| H9 | Blank | #DIV/0! | #DIV/0! | 0 |
| H10 | Blank | #DIV/0! | #DIV/0! | 0.04 |
| H11 | Blank | #DIV/0! | #DIV/0! | 0.019 |
| H12 | Blank | #DIV/0! | #DIV/0! | 0.04 |

### References

Chen, D., Liu, W., Lukianchikov, A., Hancock, G.V., Zimmerman, J., Lowe, M.G., Kim, R., Galic, Z., Irie, N., Surani, M.A., et al. (2017). Germline competency of human embryonic stem cells depends on eomesodermin. Biology of Reproduction 97, 850-861.

Chiquoine, A.D. (1954). The identification, origin, and migration of the primordial germ cells in the mouse embryo. The Anatomical Record 118, 135-146.

De Felici, M. (2016). The Formation and Migration of Primordial Germ Cells in Mouse and Man. In Results and problems in cell differentiation (Cham: Springer International Publishing), pp. 23-46.

Durruthy-Durruthy, J., Briggs, S.F., Awe, J., Ramathal, C.Y., Karumbayaram, S., Lee, P.C., Heidmann, J.D., Clark, A., Karakikes, I., Loh, K.M., et al. (2014). Rapid and efficient conversion of integration-free human induced pluripotent stem cells to GMP-grade culture conditions. PLoS ONE 9, e94231.

Ginsburg, M., Snow, M.H., and McLaren, A. (1990). Primordial germ cells in the mouse embryo during gastrulation. Development 110, 521-528.

Hayashi, K., Ohta, H., Kurimoto, K., Aramaki, S., and Saitou, M. (2011). Reconstitution of the mouse germ cell specification pathway in culture by pluripotent stem cells. Cell 146, 519-532.

Hoffman, J.A., Wu, C.-I., and Merrill, B.J. (2013). Tcf711 prepares epiblast cells in the gastrulating mouse embryo for lineage specification. Development 140, 1665-1675.

Irie, N., Weinberger, L., Tang, W.W.C., Kobayashi, T., Viukov, S., Manor, Y.S., Dietmann, S., Hanna, J.H., and Surani, M.A. (2014). SOX17 Is a Critical Specifier of Human Primordial Germ Cell Fate. Cell, 1-26.

Kattman, S.J., Witty, A.D., Gagliardi, M., Dubois, N.C., Niapour, M., Hotta, A., Ellis, J., and Keller, G. (2011). Stage-specific optimization of activin/nodal and BMP signaling promotes cardiac differentiation of mouse and human pluripotent stem cell lines. Cell Stem Cell 8, 228-240.

Kobayashi, T., and Surani, M.A. (2018). On the origin of the human germline. Development 145, dev150433.

Kobayashi, T., Zhang, H., Tang, W.W.C., Irie, N., Withey, S., Klisch, D., Sybirna, A., Dietmann, S., Contreras, D.A., Webb, R., et al. (2017). Principles of early human development and germ cell program from conserved model systems. Nature, 1-21.

Kojima, Y., Sasaki, K., Yokobayashi, S., Sakai, Y., Nakamura, T., Yabuta, Y., Nakaki, F., Nagaoka, S., Woltjen, K., Hotta, A., et al. (2017). Evolutionarily Distinctive Transcriptional and Signaling Programs Drive Human Germ Cell Lineage Specification from Pluripotent Stem Cells. Cell Stem Cell 21, 517-532.e515.

Lancaster, M.A., and Knoblich, J.A. (2014). Organogenesis in a dish: Modeling development and disease using organoid technologies. Science 345, 1247125-1247125.

Lawson, K.A., and Hage, W.J. (1994). Clonal analysis of the origin of primordial germ cells in the mouse. Ciba Foundation Symposium 182, 68-84- discussion 84-91.

Loh, K.M., Ang, L.T., Zhang, J., Kumar, V., Ang, J., Auyeong, J.Q., Lee, K.L., Choo, S.H., Lim, C Y.Y., Nichane, M., et al. (2014). Efficient Endoderm Induction from Human Pluripotent Stem Cells by Logically Directing Signals Controlling Lineage Bifurcations. Cell Stem Cell 14, 237-252.

Loh, K.M., Chen, A., Koh, P.W., Deng, T.Z., Sinha, R., Tsai, J.M., Barkal, A.A., Shen, K.Y., Jain, R., Morganti, R.M., et al. (2016). Mapping the Pairwise Choices Leading from Pluripotency to Human Bone, Heart, and Other Mesoderm Cell Types. Cell 166, 451-467.

Magnusdottir, E., and Surani, M.A. (2013). How to make a primordial germ cell. Development 141, 245-252.

Martin, R.M., Ikeda, K., Cromer, M.K., Uchida, N., Nishimura, T., Romano, R., Tong, A.J., Lemgart, V.T., Camarena, J., Pavel-Dinu, M., et al. (2019). Highly Efficient and Marker-free Genome Editing of Human Pluripotent Stem Cells by CRISPR-Cas9 RNP and AAV6 Donor-Mediated Homologous Recombination. Cell Stem Cell 24, 821-828.e825.

McGrath, K.E., Koniski, A.D., Maltby, K.M., McGann, J.K., and Palis, J. (1999). Embryonic expression and function of the chemokine SDF-1 and its receptor, CXCR4. Developmental Biology 213, 442-456.

Mitsunaga, S., Odajima, J., Yawata, S., Shioda, K., Owa, C., Isselbacher, K.J., Hanna, J.H., and Shioda, T. (2017). Relevance of iPSC-derived human PGC-like cells at the surface of embryoid bodies to prechemotaxis migrating PGCs. Proceedings of the National Academy of Sciences of the United States of America 114, E9913-E9922.

Molyneaux, K.A., Zinszner, H., Kunwar, P.S., Schaible, K., Stebler, J., Sunshine, M.J., O&apos;Brien, W., Raz, E., Littman, D., Wylie, C., et al. (2003). The chemokine SDF1/CXCL12 and its receptor CXCR4 regulate mouse germ cell migration and survival. Development 130, 4279-4286.

Novikov, N., and Evans, T. (2013). Tmem88a mediates GATA-dependent specification of cardiomyocyte progenitors by restricting WNT signaling. Development 140, 3787-3798.

O′Rahilly, R., and Müller, F. (1987). Developmental stages in human embryos (Baltimore, Maryland: Carnegie Institute of Washington).

Ohinata, Y., Ohta, H., Shigeta, M., Yamanaka, K., Wakayama, T., and Saitou, M. (2009). A Signaling Principle for the Specification of the Germ Cell Lineage in Mice. Cell 137, 571-584.

Rivera-Perez, J.A., and Magnuson, T. (2005). Primitive streak formation in mice is preceded by localized activation of Brachyury and Wnt3. Developmental Biology 288, 363-371.

Sasaki, K., Nakamura, T., Okamoto, I., Yabuta, Y., Iwatani, C., Tsuchiya, H., Seita, Y., Nakamura, S., Shiraki, N., Takakuwa, T., et al. (2016). The Germ Cell Fate of Cynomolgus Monkeys Is Specified in the Nascent Amnion. Developmental Cell 39, 169-185.

Sasaki, K., Yokobayashi, S., Nakamura, T., Okamoto, I., Yabuta, Y., Kurimoto, K., Ohta, H., Moritoki, Y., Iwatani, C., Tsuchiya, H., et al. (2015). Robust In Vitro Induction of Human Germ Cell Fate from Pluripotent Stem Cells. Cell Stem Cell 77, 178-194.

Surani, M.A., Hayashi, K., and Hajkova, P. (2007). Genetic and epigenetic regulators of pluripotency. Cell 128, 747-762.

Thomson, J.A., Itskovitz-Eldor, J., Shapiro, S.S., Waknitz, M.A., Swiergiel, J.J., Marshall, V.S., and Jones, J.M. (1998). Embryonic stem cell lines derived from human blastocysts. Science 282, 1145-1147.

Wang, P., Rodriguez, R.T., Wang, J., Ghodasara, A., and Kim, S.K. (2011). Targeting SOX17 in human embryonic stem cells creates unique strategies for isolating and analyzing developing endoderm. Cell Stem Cell 8, 335-346.

Warmflash, A., Sorre, B., Etoc, F., Siggia, E.D., and Brivanlou, A.H. (2014). A method to recapitulate early embryonic spatial patterning in human embryonic stem cells. Nature Methods 11, 847-854.

Yokobayashi, S., Okita, K., Nakagawa, M., Nakamura, T., Yabuta, Y., Yamamoto, T., and Saitou, M. (2017). Clonal Variation of Human Induced Pluripotent Stem Cells for Induction into the Germ Cell Fate. Biology of Reproduction.

Zhang, Z., Zwick, S., Loew, E., Grimley, J.S., and Ramanathan, S. (2019). Mouse embryo geometry drives formation of robust signaling gradients through receptor localization. Nature Communications 10, 4516-4514.

### P EMBODIMENTS

P Embodiment 1. A method of forming a primordial germ cell (PGC) in vitro, said method comprising:
(i) contacting a pluripotent stem cell population with a wingless integrated (WNT) agonist and a transforming growth factor beta (TGFβ) agonist, thereby forming a posterior epiblast cell population;
(ii) contacting said posterior epiblast cell population with a WNT inhibitor and removing said WNT agonist and said TGFβ agonist, thereby forming a PGC.

P Embodiment 2. A method of isolating a primordial germ cell (PGC), said method comprising:
(i) contacting a pluripotent stem cell population with a WNT agonist and a TGFβ agonist in vitro, thereby forming a posterior epiblast cell population;
(ii) contacting said posterior epiblast cell population with a WNT inhibitor and removing said WNT agonist and said TGFβ agonist; thereby forming a cell population comprising a PGC; and
(iii) separating a CXCR4+/PDGFRα-/GARP¬ cell from said cell population comprising a PGC, thereby isolating said PGC.

P Embodiment 3. The method of P embodiment 1 or 2, wherein said contacting of step (i) is for the duration of about 12 hours or less.

P Embodiment 4. The method of any one of P embodiments 1-3, wherein said PGC is formed within less than 4 days.

P Embodiment 5. The method of any one of P embodiments 1-4, wherein said contacting of step (i) comprises expanding said pluripotent stem cell population.

P Embodiment 6. The method of any one of P embodiments 1-5, wherein said contacting of step (ii) comprises expanding said posterior epiblast cell population.

P Embodiment 7. The method of any one of P embodiments 1-6, wherein said contacting of step (ii) further comprises contacting said posterior epiblast cell population with a bone morphogenetic protein (BMP), stem cell factor (SCF), epidermal growth factor (EGF) or any combination thereof.

P Embodiment 8. The method of any one of P embodiments 1-7, wherein said contacting said epiblast cell population with said WNT inhibitor, BMP, SCF, EGF or any combination thereof occurs sequentially, or simultaneously.

P Embodiment 9. The method of any one of P embodiments 1-8, wherein said pluripotent stem cell population forms a monolayer in a cell culture vessel.

P Embodiment 10. The method of any one of P embodiments 1-9, wherein said posterior epiblast cell population expresses one or more pluripotency marker genes and one or more primitive-streak marker genes.

P Embodiment 11. The method of any one of P embodiments 1-10, wherein said pluripotent stem cell population is a human pluripotent stem cell population or a human embryonic stem cell population.

P Embodiment 12. An in vitro cell composition, comprising a WNT-activated posterior epiblast cell population in a cell culture medium comprising a WNT inhibitor.

P Embodiment 13. The cell culture of P embodiment 12, wherein said cell culture medium further comprises BMP, SCF, EGF or any combination thereof.

P Embodiment 14. A method of treating infertility in a subject in need thereof, said method comprising administering a therapeutically effective amount of a PGC of any one of P embodiments 1-11 to said subject, thereby treating infertility in said subject.

### EMBODIMENTS

Embodiment 1. A method of forming a primordial germ cell (PGC) in vitro, said method comprising:
(i) contacting a pluripotent stem cell population with a wingless integrated (WNT) agonist and a transforming growth factor beta (TGFβ) agonist, thereby forming a posterior epiblast cell population;
(ii) contacting said posterior epiblast cell population with a WNT inhibitor, wherein prior to said contacting of step (ii) said WNT agonist and said TGFβ agonist are removed, thereby forming a PGC.

Embodiment 2. A method of isolating a primordial germ cell (PGC), said method comprising:
(i) contacting a pluripotent stem cell population with a WNT agonist and a TGFβ agonist in vitro, thereby forming a posterior epiblast cell population;
(ii) contacting said posterior epiblast cell population with a WNT inhibitor, wherein prior to said contacting of step (ii) said WNT agonist and said TGFβ agonist are removed, thereby forming a cell population comprising a PGC; and
(iii) separating a CXCR4+/PDGFRα-/GARP¬ cell from said cell population comprising a PGC, thereby isolating said PGC.

Embodiment 3. The method of embodiment 1 or 2, wherein said contacting of step (i) is for the duration of about 12 hours or less.

Embodiment 4. The method of any one of embodiments 1-3, wherein said PGC is formed within less than 4 days.

Embodiment 5. The method of any one of embodiments 1-4, wherein said contacting of step (i) comprises expanding said pluripotent stem cell population.

Embodiment 6. The method of any one of embodiments 1-5, wherein said contacting of step (ii) comprises expanding said posterior epiblast cell population.

Embodiment 7. The method of any one of embodiments 1-6, wherein said contacting of step (ii) further comprises contacting said posterior epiblast cell population with a bone morphogenetic protein (BMP), stem cell factor (SCF), epidermal growth factor (EGF) or any combination thereof.

Embodiment 8. The method of any one of embodiments 1-7, wherein said contacting said epiblast cell population with said WNT inhibitor, BMP, SCF, EGF or any combination thereof occurs sequentially, or simultaneously.

Embodiment 9. The method of any one of embodiments 1-8, wherein said pluripotent stem cell population forms a monolayer in a cell culture container.

Embodiment 10. The method of any one of embodiments 1-9, wherein said contacting of step (i) and step (ii) occurs in serum-free medium.

Embodiment 11. The method of any one of embodiments 1-9, wherein said posterior epiblast cell population expresses one or more pluripotency marker genes and one or more primitive-streak marker genes.

Embodiment 12. The method of any one of embodiments 1-11, wherein said pluripotent stem cell population is a human pluripotent stem cell population or a human embryonic stem cell population.

Embodiment 13. A primordial germ cell formed by a method of any one of embodiments 1-12.

Embodiment 14. An in vitro cell culture, comprising a WNT-activated posterior epiblast cell population in a cell culture medium comprising a WNT inhibitor.

Embodiment 15. An in vitro cell culture, comprising a CXCR4+/PDGFRα-/GARP¬ PGC in a cell culture medium comprising a Wnt inhibitor.

Embodiment 16. The cell culture of embodiment 15, wherein said PGC expresses one or more of the proteins selected from OCT4, NANOG, TFCP2L1, BLIMP1, NANOS3 and TFAP2C (AP2γ).

Embodiment 17. The cell culture of embodiment 15 or 16, wherein said PGC does not express detectable amounts of FOXA2, HHEX or extraembryonic fate (CDX2).

Embodiment 18. The cell culture of any one of embodiments 14-17 wherein said cell culture medium further comprises BMP, SCF, EGF or any combination thereof.

Embodiment 19. The cell culture of any one of embodiments 14-18, wherein said cell culture is in a cell culture container.

Embodiment 20. The cell culture of any one of embodiments 14-19, wherein said cell culture forms a monolayer.

Embodiment 21. The cell culture of any one of embodiments 14-19, wherein said cell culture medium is a serum-free medium.

Embodiment 22. A primordial germ cell (PGC) in a cell culture container comprising a cell culture medium comprising a WNT inhibitor, wherein said PGC is a CXCR4+/PDGFRα-/GARP¬ PGC.

Embodiment 23. The primordial germ cell of embodiment 22, wherein said PGC expresses one or more of the proteins selected from OCT4, NANOG, TFCP2L1, BLIMP1, NANOS3 and TFAP2C (AP2γ).

Embodiment 24. The primordial germ cell of embodiment 22 or 23, wherein said PGC does not express detectable amounts of FOXA2, HHEX or extraembryonic fate (CDX2).

Embodiment 25. The primordial germ cell of any one embodiment 22-24,wherein said cell culture medium further comprises BMP, SCF, EGF or any combination thereof.

Embodiment 26. The primordial germ cell of any one embodiment 22-25, wherein said PGC forms part of a cell monolayer.

Embodiment 27. The primordial germ cell of any one embodiment 22-26, wherein said cell culture medium is a serum-free medium.

Embodiment 28. A method of treating infertility in a subject in need thereof, said method comprising administering a therapeutically effective amount of a PGC of any one of embodiments 13 or 22-27 to said subject, thereby treating infertility in said subject.

1. A method of forming a primordial germ cell (PGC) in vitro, said method comprising:
(i) contacting a pluripotent stem cell population with a wingless integrated (WNT) agonist and a transforming growth factor beta (TGFβ) agonist, thereby forming a posterior epiblast cell population; and
(ii) contacting said posterior epiblast cell population with a WNT inhibitor, wherein prior to said contacting of step (ii) said WNT agonist and said TGFβ agonist are removed, thereby forming a PGC.

2. A method of isolating a primordial germ cell (PGC), said method comprising:
(i) contacting a pluripotent stem cell population with a WNT agonist and a TGFβ agonist in vitro, thereby forming a posterior epiblast cell population;
(ii) contacting said posterior epiblast cell population with a WNT inhibitor, wherein prior to said contacting of step (ii) said WNT agonist and said TGFβ agonist are removed, thereby forming a cell population comprising a PGC; and
(iii) separating a CXCR4⁺/PDGFRα⁻/GARP cell from said cell population comprising a PGC, thereby isolating said PGC.

3. The method of claim 1, wherein said contacting of step (i) is for the duration of about 12 hours or less.

4. The method of claim 1, wherein said PGC is formed within less than 4 days.

5. The method of claim 1, wherein
a) said contacting of step (i) comprises expanding said pluripotent stem cell population; and/or
b) said contacting of step (ii) comprises expanding said posterior epiblast cell population.

6. (canceled)

7. The method of claim 1, wherein said contacting of step (ii) further comprises contacting said posterior epiblast cell population with a bone morphogenetic protein (BMP), stem cell factor (SCF), epidermal growth factor (EGF) or any combination thereof.

8. The method of claim 1, wherein said contacting said epiblast cell population with said WNT inhibitor, BMP, SCF, EGF or any combination thereof occurs sequentially, or simultaneously.

9. The method of claim 1, wherein said pluripotent stem cell population forms a monolayer in a cell culture container.

10. The method of claim 1, wherein said contacting of step (i) and step (ii) occurs in serum-free medium.

11. The method of claim 1, wherein said posterior epiblast cell population expresses one or more pluripotency marker genes and one or more primitive-streak marker genes.

12. The method of claim 1, wherein said pluripotent stem cell population is a human pluripotent stem cell population or a human embryonic stem cell population.

13. A primordial germ cell formed by a method of claim 1.

14. An in vitro cell culture, comprising i) a WNT-activated posterior epiblast cell population; or 2) a CXCR4⁺/PDGFRα⁻/GARP primordial germ cell (PGC), in a cell culture medium comprising a WNT inhibitor.

15. (canceled)

16. The cell culture of claim 14, wherein said PGC expresses one or more of the proteins selected from OCT4, NANOG, TFCP2L1, BLIMP1, NANOS3 and TFAP2C (AP2γ).

17. The cell culture of claim 14, wherein said PGC does not express detectable amounts of FOXA2, HHEX or extraembryonic fate (CDX2).

18. The cell culture of claim 14 wherein said cell culture medium further comprises BMP, SCF, EGF or any combination thereof.

19. (canceled)

20. The cell culture of claim 14, wherein said cell culture forms a monolayer.

21. The cell culture of claim 14, wherein said cell culture medium is a serum-free medium.

22-27. (canceled)

28. A method of treating infertility in a subject in need thereof, said method comprising administering a therapeutically effective amount of a PGC of claim 13 to said subject, thereby treating infertility in said subject.
